(19) United States
(12) Patent Application Publication
(10) Pub. No.: US 2024/0175032 A1
(43) Pub. Date: May 30, 2024

(54) COMPOSITIONS AND METHODS FOR INHIBITING EXPRESSION OF PCSK9

(71) Applicant: Sirnaomics, Inc., Germantown, MD (US)

(72) Inventors: Xiaoyong LU, Germantown, MD (US); David M. EVANS, Germantown, MD (US); Patrick Y. LU, Germantown, MD (US)

(21) Appl. No.: 18/179,410

(22) Filed: Mar. 7, 2023

Related U.S. Application Data
(63) Continuation of application No. PCT/US22/11489, filed on Jan. 6, 2022.
(60) Provisional application No. 63/134,562, filed on Jan. 6, 2021.

(51) Int. Cl. C12N 15/113; A61K 31/713; A61K 47/54; A61K 47/64; A61P 3/06

(52) U.S. Cl. CPC C12N 15/1137; A61K 31/713; A61K 47/549; A61K 47/6455; A61P 3/06; C12N 2310/14; C12N 2310/315; C12N 2310/3513; C12N 2310/52; C12N 2320/32

(57) ABSTRACT

Double stranded RNAi agents for inhibiting PCSK9 gene expression are provided. Complexes in which the siRNA agents are covalently conjugated to a peptide docking vehicle (PDoV), and further covalently linked to one or more targeting ligands also are provided. Pharmaceutical compositions containing the RNAi agents and complexes are provided, together with methods for their use.

### CROSS REFERENCE TO RELATED APPLICATIONS

This application is a continuation of International Application No. PCT/US22/11489, filed Jan. 6, 2022, claiming priority under 35 USC 119 (e) to provisional application No. 63/134,562 filed Jan. 6, 2021, each of which is incorporated herein by reference in its entirety.

### SEQUENCE LISTING

The instant application contains a Sequence Listing which has been submitted electronically in XML file format and is hereby incorporated by reference in its entirety. Said XML copy, created on August 16, 2023, is named 4690_0039C_SL.xml and is 943,712 bytes in size.

### FIELD

Double stranded RNAi agents for inhibiting PCSK9 gene expression are provided. Complexes in which the siRNA agents are covalently conjugated to a peptide docking vehicle (PDoV), and further covalently linked to one or more targeting ligands also are provided. Pharmaceutical compositions containing the RNAi agents and complexes are provided, together with methods for their use.

### BACKGROUND

### Protein Convertase Subtilisin Kexin 9 (PCSK9)

Protein convertase subtilisin kexin 9 (PCSK9) is the ninth member of the mammalian family of serine proteinases, a group of protein convertases (PCs) that cleave inactive secretory precursors into bioactive proteins and peptides (Seidah et al., 2003, Proc. Natl. Acad. Sci. U.S.A. 100, 928-933). PCSK9 was first identified in 2003 in primary cerebellar neurons as an mRNA upregulated during apoptosis (Id.). Originally called neural apoptosis-regulated convertase-1 (NARC-1). PCSK9 mainly interacts with low-density lipoprotein (LDL) (Kosenko et al., J. Bio. Chem. 288, 8279-8288 (2013); Ferri et al., Atherosclerosis, 253:214-224 (2016); Burnap et al., JAm Coll Cardiol. 75:1495-1497 (2020)). PCSK9 has been shown to have a vital role in cholesterol metabolism, by regulating circulating low-density lipoprotein (LDL) through the hepatic LDL receptor degradation pathway. PCSK9 mRNA can be down regulated (Maxwell, K. N. (2003) J. Lipid Res. 44. 2109-2119), and upregulated in an in vivo mice model (Horton, J. D., Proc. Natl. Acad. Sci. USA 100, 12027-12032 (2003)). Overexpression studies point to a role for PCSK9 in controlling LDLR levels and, hence, LDL uptake by the liver (Maxwell, K. N., Proc.Natl. Acad. Sci. USA 101, 7100-7105 (2004)), Benjannet, S., et al. (2004). J. Biol. Chem. 279, 48865-48875, Park, S.W., J. Biol. Chem. 279, 50630-50638 (2004)).

PCSK9 is also known as FH3, HCHOLA3, NARC-1, or NARC1. The term PCSK9 includes human PCSK9, the amino acid and nucleotide sequence of which may be found in, for example, GenBank Accession No. GI:299523249; mouse PCSK9, the amino acid and nucleotide sequence of which may be found in, for example, GenBank Accession No. GI:163644257; rat PCSK9, the amino acid and nucleotide sequence of which may be found in, for example, GenBank Accession No. GI:77020249. Additional examples of PCSK9 mRNA sequences are readily available using, e.g., GenBank.

The most prominent role of PCSK9 is its interaction with the low-density lipoprotein receptor (LDLR) in the liver (Abifadel et al., Nat Genet 34:154-6 (2003)). When an LDL particle with PCSK9 binds to an LDLR, the catalytic domain of PCSK9 interacts with the epidermal growth factor-like repeat A (EGF-A) domain of the LDLR. The low pH of the endosome enhances PCSK9/LDLR affinity when the complex is endocytosed, and PCSK9 prevents the open extended conformation of LDLR associated with receptor recycling. Instead, the PCSK9/LDLR complex is shuttled to the lysosome for degradation, resulting in fewer surface LDLRs and higher plasma cholesterol levels (Seidah et al., supra). Regulation of plasma PCSK9, LDLR, and LDL-C levels is tightly linked because PCSK9 is cleared from the plasma mainly by binding to LDLR but at the same time induces LDLR degradation due to its interaction (Tavori et al., Circulation, 127:2403-13, (2013)).

High levels of cholesterol and in particular high levels of low-density lipoprotein (LDL; >4.1 mmol/L or 160 mg/dL), are directly linked with an increased risk of cardiovascular disease. Severe hypercholesterolemia that cannot be sufficiently controlled by statins is caused by mutations in the hepatic low-density lipoprotein receptor (LDLR) that decrease or abolish LDLR-mediated removal of LDL particles from the bloodstream (Goldstein JL, et al., J. Biol. Chem. 1974, 249, 5153.). This genetic disease is called familial hypercholesterolemia (FH) and can also be caused by mutations of apolipoprotein B, the main protein component of LDL particles which facilitates binding of LDL particles to LDLR. When PCSK9 protein is bound to LDL-loaded LDLR during endocytosis, the complex is directed to the lysosome for degradation, while LDL-loaded LDLR without PCSK9 offloads the LDL particle and is then returned to the cell surface. Thus, gain-of-function PCSK9 mutations result in increased degradation of LDLR and reduced uptake of LDL particles from the bloodstream. Accumulation of LDL cholesterol in the bloodstream accelerates the progress of atherosclerosis and leads to premature death from cardiovascular events due to atherosclerotic lesion rupture.

The discovery in 2003 by Abifadel et al. linked mutations in the gene encoding PCSK9 with autosomal dominant hypercholesterolemia (ADH). Major findings over the last decade have revealed the following: a. Gain-of-function mutations in PCSK9 are a cause of

ADH; b. Loss-of-function mutations in PCSK9 are associated with low LDL-C levels and markedly reduced cardiovascular risk. Loss of function mutations in PCSK9 have been studied in mouse models (Rashid et al., (2005) PNAS, 102, 5374-5379), and identified in human individuals (Cohen et al. (2005) Nature Genetics 37:161-165). In both cases loss of PCSK9 function leads to lowering of total and LDLc cholesterol. In a retrospective outcome study in past decades, loss of one copy of PCSK9 was shown to shift LDLc levels lower and to lead to an increased risk-benefit protection from developing cardiovascular heart disease (Cohen et al., N. Engl. J. Med., 354:1264-1272 (2006)).

After the discovery of the positive effects of PCSK9 loss-of-function mutations, two fully humanized monoclonal antibodies against PCSK9 were developed: alirocumab (Praluent) by Regeneron in partnership with Sanofi and evolocumab (Repatha) by Amgen. However, PCSK9 inhibition could not reduce LDL cholesterol in LDLR-negative homozygous FH patients.

### siRNA Therapeutics in Treatment of LDL and Other Disease

Double-stranded RNA has been shown to silence gene expression via RNA interference (RNAi). Short-interfering RNA (siRNA)-induced RNAi regulation shows great potential to treat a wide variety of human diseases from cancer to other traditional undruggable disease. Onpattro (patisiran) infusion is approved for the treatment of peripheral nerve disease (polyneuropathy) caused by hereditary transthyretin-mediated amyloidosis (hATTR) in adult patients. This is the first FDA approved siRNA based drug treatment for patients with polyneuropathy caused by hATTR, a rare, debilitating and often fatal genetic disease characterized by the buildup of abnormal amyloid protein in peripheral nerves, the heart and other organs.

The delivery of an iRNA agent in the disclosed embodiments to a cell e.g., a cell within a subject, such as a human subject (e.g., a subject in need thereof, such as a subject having a lipid disorder, such as a hyperlipidemia) can be achieved in a number of different ways. For example, delivery may be performed by contacting a cell with an iRNA in the disclosed embodiments either in vitro or in vivo. In vivo delivery may also be performed directly by administering a composition comprising an iRNA, e.g., a dsRNA, to a subject. Alternatively, in vivo delivery may be performed indirectly by administering one or more vectors that encode and direct the expression of the iRNA. These alternatives are discussed further below.

In general, any method of delivering a nucleic acid molecule (in vitro or in vivo) can be adapted for use with an iRNA of the disclosed embodiments (see e.g., Akhtar S. and Julian R L. (1992) Trends Cell. Biol. 2(5):139-144 and WO94/02595). For in vivo delivery, factors to consider in order to deliver an iRNA molecule include, for example, biological stability of the delivered molecule, prevention of non-specific effects, and accumulation of the delivered molecule in the target tissue. The non-specific effects of an iRNA can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the iRNA molecule to be administered. Several studies have shown successful knockdown of gene products when an iRNA is administered locally. For example, intraocular delivery of a VEGF dsRNA by intravitreal injection in cynomolgus monkeys (Tolentino, M J., et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of a dsRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc.

Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55). For administering an iRNA systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the iRNA composition to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an iRNA to an aptamer has been shown to inhibit tumor growth and mediate tumor regression in a mouse model of prostate cancer (McNamara, J O., et al (2006) Nat. Biotechnol. 24:1005-1015). In an alternative embodiment, the iRNA can be delivered using drug delivery systems such as a nanoparticle, a dendrimer, a polymer, liposomes, or a cationic delivery system. Positively charged cationic delivery systems facilitate binding of an iRNA molecule (negatively charged) and also enhance interactions at the negatively charged cell membrane to permit efficient uptake of an iRNA by the cell. Cationic lipids, dendrimers, or polymers can either be bound to an iRNA, or induced to form a vesicle or micelle (see e.g., Kim S H., et al (2008) Journal of Controlled Release 129(2):107-116) that encases an iRNA. The formation of vesicles or micelles further prevents degradation of the iRNA when administered systemically. Methods for making and administering cationic-iRNA complexes are well within the abilities of one skilled in the art (see e.g., Sorensen, D R., et al (2003) J. Mol. Biol 327:761-766; Verma, U N., et al (2003) Clin. Cancer Res. 9:1291-1300; Arnold, A Set al (2007) J. Hypertens. 25:197-205. Some non-limiting examples of drug delivery systems useful for systemic delivery of iRNAs include DOTAP (Sorensen, D R., et al (2003), supra; Verma, U N., et al (2003), supra), Oligofectamine, “solid nucleic acid lipid particles” (Zimmermann, T S., et al (2006) Nature 441:111-114), cardiolipin (Chien, P Y., et al (2005) Cancer Gene Ther. 12:321-328; Pal, A., et al (2005) Int J. Oncol. 26:1087-1091), polyethyleneimine (Bonnet M E., et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A., et al (2007) Biochem. Soc. Trans. 35:61-67; Yoo, H., et al (1999) Pharm. Res. 16:1799-1804). In some embodiments, an iRNA forms a complex with cyclodextrin for systemic administration. Methods for administration and pharmaceutical compositions of iRNAs and cyclodextrins can be found in U.S. Pat. No. 7,427,605.

In addition, it was discovered that tri-antennary N-acetylgalactosamine (GalNAc) could mediate highly efficient targeted delivery of siRNAs to hepatocytes via binding to the asialoglycoprotein receptor. siRNA delivery to the liver can be achieved using ASGPR-targeted GalNAc-siRNA conjugates due to the properties of ASGPR that are well suited for macromolecular drug deliver to hepatocytes. In particular, hepatocytes express millions of copies of ASGPR on their cell surface, which cycle at a rapid rate of every 10-15 min. These properties make a GalNAc-based delivery approach effective. Currently, the most advanced RNAi therapeutic approved by FDA to market taking chemical modifications with GalNAc delivery is Alnylam's Givlaari (givosiran) for acute hepatic porphyria (AHP), a rare inherited genetic disease. It binds to and suppresses the translation of delta aminolevulinic acid synthase 1 (ALAS1) mRNA, thereby reducing the neurotoxic intermediates in this disease. On Nov. 19, 2020, Alnylam received approval for OXLUMO™ (lumasiran) in the European Union for the Treatment of Primary Hyperoxaluria Type 1 in All Age Groups. OXLUMO is the first therapeutic approved for the treatment of PH1, and the only therapy proven to lower harmful oxalate levels that drive the progression of PH1 disease.

Another siRNA drug approved by European regulation in 2020 is Inclisiran (ALN-PCSsc; developed by Alnylam Pharmaceuticals and licensed to The Medicines Company, later sold to Novartis) for the treatment of hypercholesterolemia. It binds to and cleaves the mRNA sequence of proprotein convertase subtilisin kexin type 9 (PCSK9), which is a target to lower the level of low-density lipoprotein (LDL) cholesterol. There are more than 30 clinical trial going on with RNAi related drug development.

Thus, this gene silence mechanism is now revolutionizing the development of a new pharmaceutical therapeutic for treating undrugable disease and disorders that are caused by the unwanted regulation of a gene.

### SUMMARY

What is provided is a chemical construct containing a Peptide Docking Vehicle (PDoV) covalently linked to (a) a targeting moiety, and (b) a therapeutic nucleic acid, where the therapeutic nucleic acid inhibits expression of PCSK9 gene. The PDoV may contain multiple repeating units of histidine and lysine and/or the targeting moiety may bind to the asialoglycoprotein receptor. The oligonucleotide may contain an siRNA, an antisense oligonucleotide, a miRNA, an aptamer, a decoy oligonucleotide, or a CpG motif, and advantageously may be an siRNA selected from the group consisting of the molecules shown in Table 1 and Table 2.

The PDoV construct may contain an endosomal release motif that contains at least two targeting moieties and/or at least one therapeutic oligonucleotide.

The PDoV advantageously has structure I or II, where A and B are independently a peptide sequence of H, K, R, HH, HHH, HHHH (SEQ ID NO: 1), FMK, HHHK (SEQ ID NO: 2), D is an siRNA, R_{L} is a targeting ligand, and R_{S} is a covalent linker to the nucleic acid

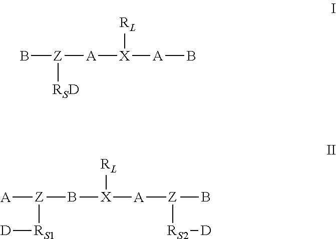

The PDoV peptide construct may have a structure selected from the group consisting of PDoV 1 (SEQ ID NO: 130), PDoV 2 (SEQ ID NO: 5), PDoV 3 (SEQ ID NO: 131), PDoV 3a (SEQ ID NO: 132), and PDoV 4 (SEQ ID NO: 133):

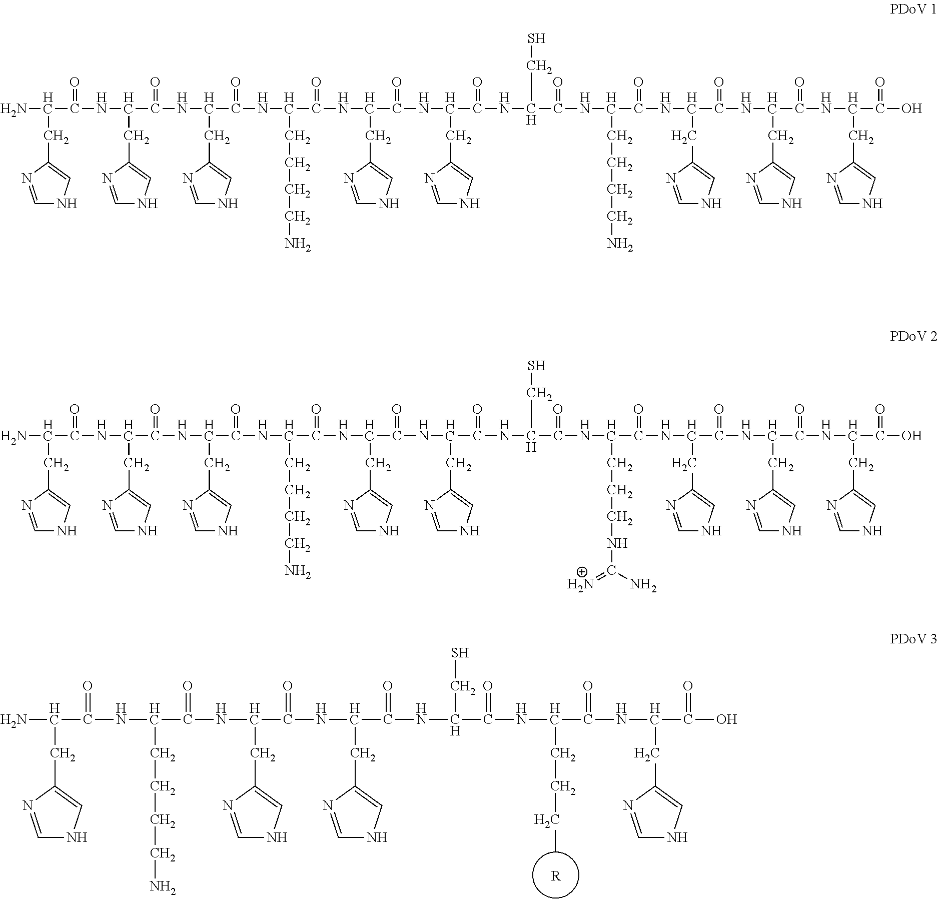

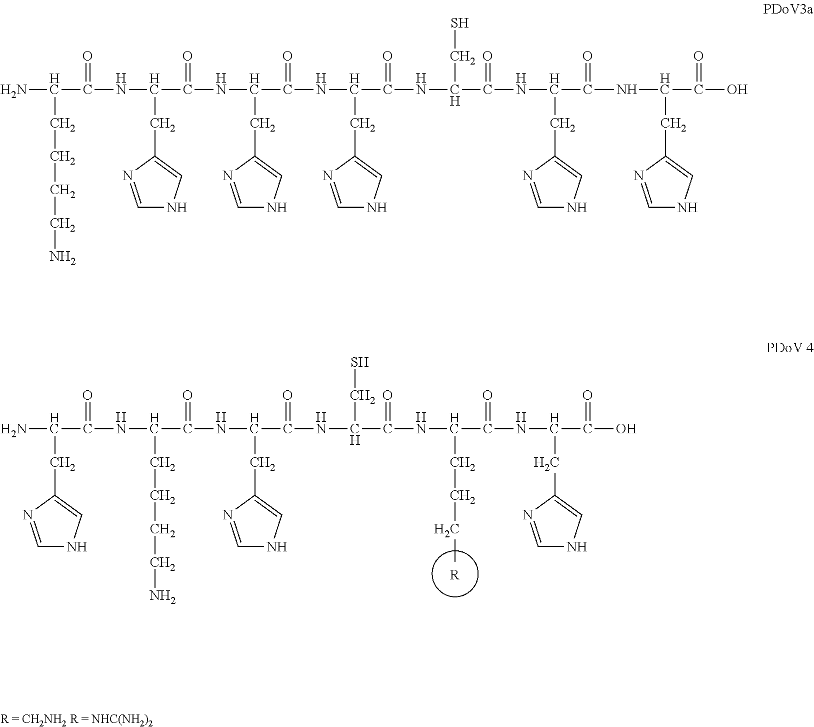

The targeting moiety may contain a ligand covalently linked to the PDoV via a linker of formula III or IV, where n is 1-3:

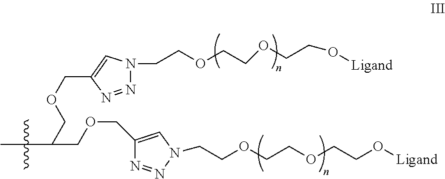

where n is 1, 2, or 3; or

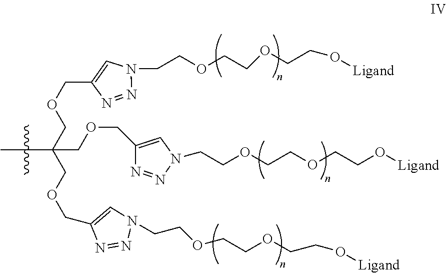

where n is 1, 2, or 3. The linker between the targeting ligand and the PDoV peptide may contain a polyethylene glycol chain —(CH₂CH₂O)ₙ—, or an alkylene chain —(CH₂CH₂)ₙ— chain, where n is an integer from 2-15. Rs may be a bioorthogonal reactive moiety that links the nucleic acid to the PDoV peptide, where the reactive moiety is selected from the group consisting of an amine, hydrazine, N-hydroxysuccinimide, azido, alkyne, carboxylic acid, thiol, maleimide, and phosphine diester.

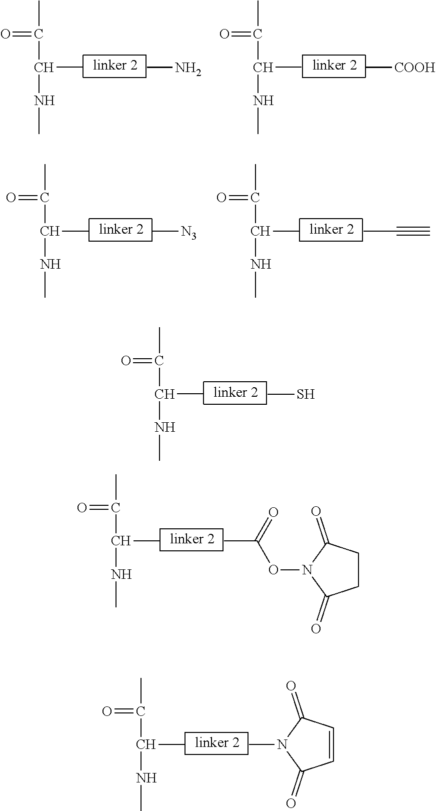

In these constructs, the siRNA molecule may contain a duplex of two complimentary, single-stranded oligonucleotides, where the oligonucleotides are the same length, and each has a length of 10-29 bases. The single-stranded oligonucleotides in the duplex may have a length of 19-27 bases. The nucleotides may contain, for example, deoxyribonucleotides and/or ribonucleotides. In some embodiments the siRNA molecule may contain at least one nucleotide chemically modified at the 2′ position, for example, a chemically modified nucleotide selected from the group consisting of 2′-O-methyl, 2′-fluoro, 2′-O-methoxyethyl and 2′-O-allyl:

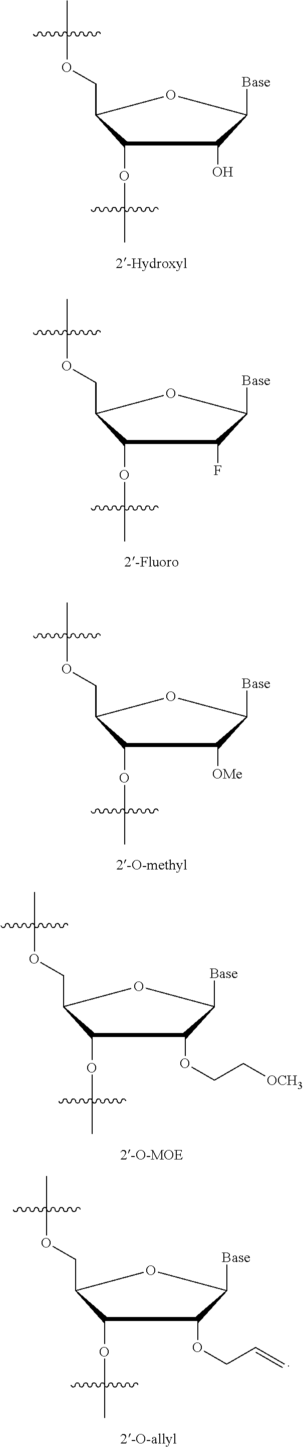

In further embodiments the siRNA molecule may contain one or more chemically modified nucleotides selected from the group consisting of a phosphorothioate diester or phosphorodithioate diester.

The therapeutic nucleic acid may be an siRNA that targets the PCSK9 gene selected from the group consisting of the RNA molecules of Table 1 and Table 2. The construct may further contain a second siRNA molecule that targets the PCSK9 gene. One or both siRNA molecules may have a sequence selected from the group consisting of the sequences of Table 1 and Table 2.

In some embodiments the therapeutic nucleic acid may be covalently linked by a linker to the PDoV via the 5′ or 3′ position of a nucleotide or nucleoside in the nucleic acid. The linker may be, for example, an aliphatic chain, a polyethylene glycol chain, like hexanol ethylene glycol, or other hydrophobic lipid (hexanal —C₆H₁₃—) chain.

In some embodiments the targeting ligand may be selected from the group consisting of N-acetyl-galactosamine (GalNAc), galactose, galactosamine, N-formal-galactosoamine, N-propionyl-galactosamine, and N-butanoylgalactosamine.

In further embodiments the PDoV contains a cysteine.

The construct may have the structure (SEQ ID NO: 134):

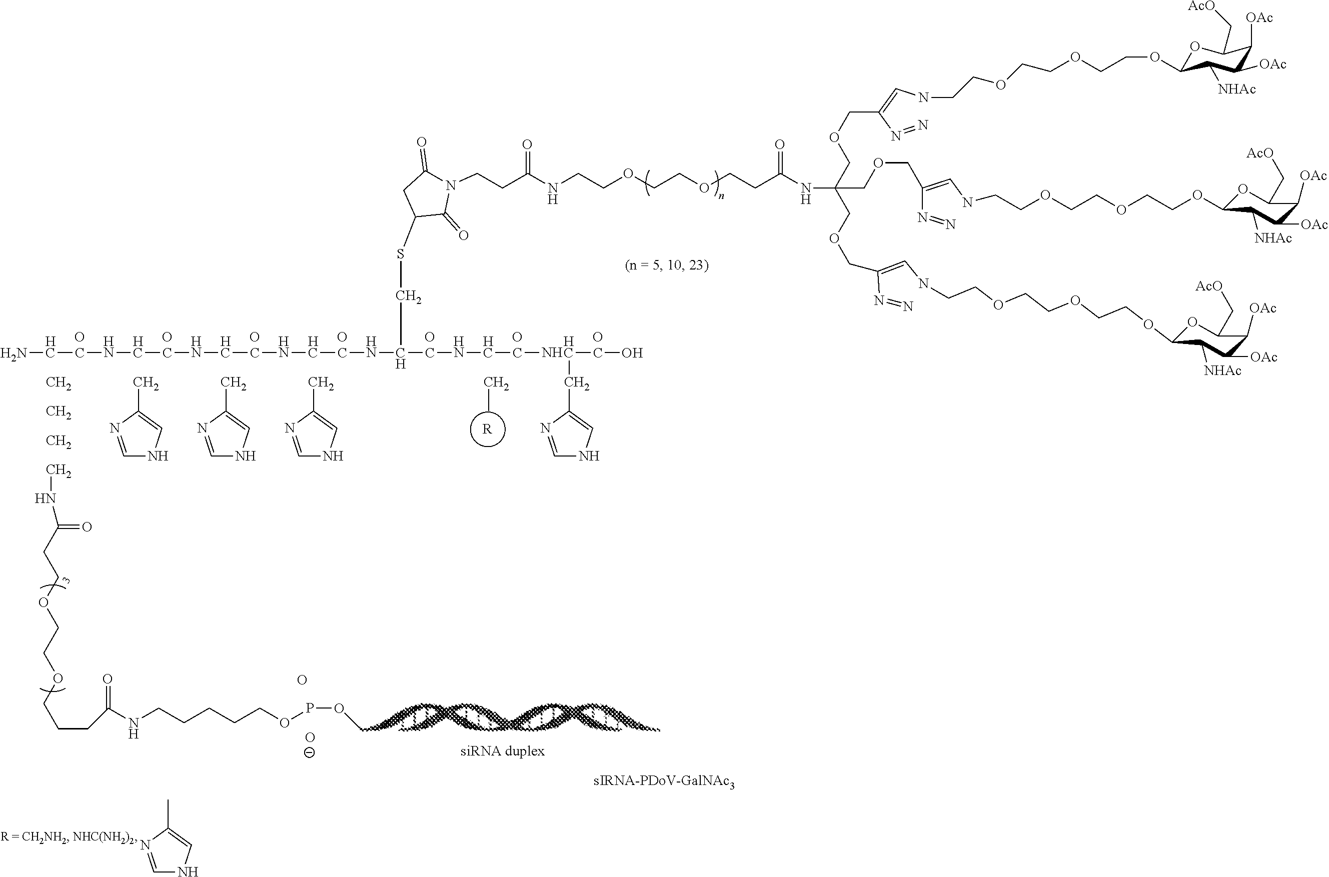

The construct may have the structure (SEQ ID NO: 135):

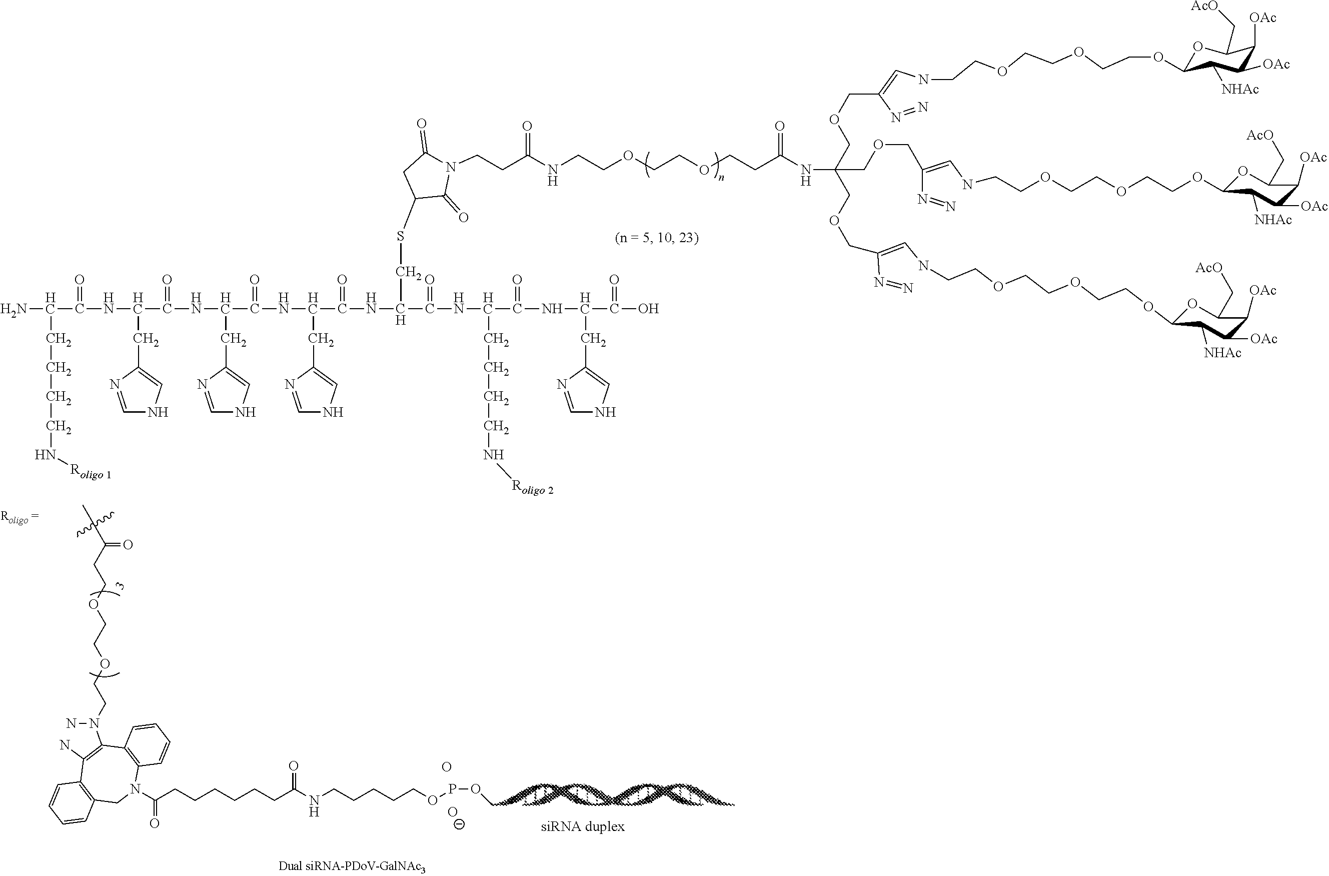

Also provided are pharmaceutical compositions containing a construct as described above and a pharmaceutically acceptable carrier. The pharmaceutically acceptable carrier may contain, for example, water and one or more salts or buffers selected from the group consisting of potassium phosphate monobasic anhydrous NF, sodium chloride USP, sodium phosphate dibasicheptahydrate USP, glucose, and Phosphate Buffered Saline (PBS).

Further provided are methods of lowering the serum LDL cholesterol or treating PCSK9 gene related cancer in a subject, by administering to the described subject a pharmaceutical composition as described above. The subject may be a primate, for example a human.

### BRIEF DESCRIPTION OF THE FIGURES

FIG. 1 shows a schematic representation of the design of the [GalNAc] Peptide Docking Vehicle (G-PDoV). Trivalent GalNAc was covalently conjugated on one docking site A. Oligonucleotide or siRNA was conjugated on the other one or two docking sites B respectively.

FIG. 2 shows the design of the Peptide Docking Vehicle (PDoV): it has a (HₙKₘ)ₒXₚZ_{q} (SEQ ID NO: 3) peptide backbone with multiple repeating units of histidine (H), lysine (K) and functional units X (amino acid, or functional linker), where=1-10, m=1-10, o=1-10, p=1-5, q=1-5. HK repeating units have been shown to have good cell penetrating ability, and to facilitate endosome release. The lysine or the various functional unit X or Z act as the docking sites for the conjugation of ligands and Z acts as the docking sites for the conjugation of oligonucleotide through a different covalent linkage. For example, the site \textcircled{1} will only be able to react in the presence of ligand such as GalNAc or other targeting ligands. The site \textcircled{3} can only conjugate with oligonucleotide and siRNA.

FIG. 3 shows the structure of the PDoV construct. The PDoV construct is a cell penetrating/endosome releasing peptide inserted with multiple conjugation sites X and Z. Site X is used to conjugate the targeting ligand, and site Z is used to conjugate multiple oligonucleotide or nucleic acid. Some construct examples for the PDoV include where: A represents a peptide sequence K, R, H, HH, HHH, HHHH (SEQ ID NO: 1), HHK, HHHK (SEQ ID NO: 2) or other short peptide; B represents a peptide sequence K, R, H, HH, HHH, HHHH (SEQ ID NO: 1), HHK, HHHK (SEQ ID NO: 2), or other short peptide, other amino acid or combination; D represents oligonucleotide, siRNA, mRNA, aptamer; R_{L} represents ligand; and R_{S} represents linker to oligonucleotide.

FIG. 4 shows an example of the structure of PDoV, containing one or two oligonucleotide sites and one ligand conjugation site. Figure discloses SEQ ID NOS 130, 130, 5, 138-139, 140-141, 133, 142 and 132, respectively, in order of appearance.

FIG. 5 shows an example of the structure of a second generation PDoV, containing two oligonucleotide sites and one multivalent ligand conjugation site. FIG. 5 discloses SEQ ID NO: 11.

FIG. 6 shows an example of alternative structure of a PDoV, containing two oligonucleotide sites and multi-ligand conjugation sites. The ligands can be conjugated individually one by one on the PDoV backbone. FIG. 6 discloses SEQ ID NO: 12.

FIG. 7 shows linkage selection for the conjugation sites. Chemical group R_{S} represents a “click” like reactive moiety to conjugate the oligonucleotide with the PDoV peptide vehicle. The reactive moiety can be amine, hydrazine, N-hydroxysuccinimide, azido, alkyne, carboxylic acid, thiol, maleimide, or other chemical reactive moiety known in the art

FIG. 8 shows representative examples of the linkage reactive site.

FIG. 9 shows how the linker 2 in the conjugation R-linker2-siRNA is a chemical spacer between the peptide and the conjugation site, allowing the conjugation site to be attached at the linker's terminal site. The linker 2 can be an aliphatic chain or a polyethylene glycol chain, or other hydrophobic lipid or hydrophilic chain. The group 2 at the end site is the reactive site for the chemical conjugation with the siRNA end.

FIG. 10 shows an example of the linkage selection for the ligand conjugation sites. The linkage for the ligand conjugation R_{L} can be selected from FIG. 11.

FIG. 11 shows examples of R_{L} GalNAc molecule: monovalent GalNAc molecule, bivalent GalNAc molecule and trivalent GalNAc molecule. The conjugation site can be maleimide/thiol or may be selected from the Group 2 list shown in FIG. 9.

FIG. 12 shows a representative example of the construction of the siRNA-PDoV-ligand compound 1. Figure discloses SEQ ID NO: 143.

FIG. 13 shows a representative example of construction of the siRNA-PDoV-ligand compound 2. Figure discloses SEQ ID NO: 144.

FIG. 14 shows a representative example of construction of the dual PCSK9 siRNA-PDoV-ligand compound 3. Figure discloses SEQ ID NO: 145.

FIG. 15. Knockdown efficacy study of unmodified PCSK9 siRNA. The in vitro experiment was done in HepG2 cells, 1×10⁵ per well in 12 well plate, siRNA final concentration is 5μM. Transfection duration was 24 hr. There are 11 samples, plus one NS control, no Blank in this setup. QRTPCR: HPRT (as internal control) and PCSK9 primer (F+R, 20 μM each, 0.2 μL per reaction) and probe (10 μM, 0.4 μL per reaction). All nine PCSK9 siRNA showed significant silencing comparing to Lipo NS.

FIG. 16. Knockdown efficacy study of PCSK9 siRNA. The in vitro experiment was done in HepG2 cells, 1×105 per well in 12 well plate, siRNA final concentration is 50 μM. Transfection duration was 24 hr. There are 11 samples, plus one NS control, no Blank in this setup. QRTPCR: HPRT (as internal control) and PCSK9 primer (F+R, 20 μM each, 0.2 μL per reaction) and probe (10 μM, 0.4 μL per reaction). PCSKd3 siRNA showed significant silencing comparing to Lipo NS.

FIG. 17. Knockdown efficacy study of PCSK9 siRNA. The in vitro experiment was done in HepG2 cells, 1×10⁵ per well in 12 well plate, siRNA final concentration is 50 μM. Transfection duration was 24 hr. There are 11 samples, plus one NS control, no Blank in this setup. QRTPCR: HPRT (as internal control) and PCSK9 primer (F+R, 20 μM each, 0.2 μL, per reaction) and probe (10 μM, 0.4 μM per reaction). All nine PCSK9 siRNA showed significant silencing comparing to Lipo NS.

FIG. 18. Knockdown efficacy study of PCSK9 siRNA by Serial dilution. In vitro knockdown experiment was performed in HepG2 cell line in 2×10⁵ cells per well in 12 well plate. siRNA final concentrations were used as 10 nM, 10× dilution (e.g. 10 nm, 1 nm, 0.1 nm, and 0.01 nm) Cells were incubated for 24hr in transfection. Multiplex PCR conditions Ratio for PCS:HPRT primer was 4:1 (800 nm:400 nm).

FIG. 19. Knockdown efficacy study of Modified PCSK9 siRNA. HepG2: 2×105 cells per well in 12 well plate in transfection time for 24 hr. cDNA was 30 ng in 8.2 μL. PCS siRNA concentrations in transfection: 10 nM, 1 nM, 0.1 nM, and 0.01 nM, 10× serial dilution. Lipofectamine RNAi Max 2μL/Rxn in 98 ul OptiMEM; siRNA was applied in 100 μL of OptiMEM; then mixed together and incubate for 15 min. Multiplex PCR was HPRT probe (10 μM), 0.4 μL). HPRT Primer used as (F+R, 20 uM each), 0.2 μL (final concentration is 200 nM). PCS probe was (10 μM, 0.4 μL). PCS Primer (F+R, 20 uM each), 0.8 ul (final concentration is 800 nM). TaqPath 2× master mix was used as 10 μL.

FIG. 20. Knockdown efficacy study of Modified PCSK9 siRNA. HepG2: 2×105 cells per well in 12 well plate in transfection time for 24 hr. cDNA was 30 ng in 8.2 μL. PCS siRNA concentrations in transfection: 10 nM, 1 nM, 0.1 nM, and 0.01 nM, 10× serial dilution. Lipofectamine RNAi Max 2 μL/Rxn in 98 ul OptiMEM; siRNA was applied in 100 μL of OptiMEM; then mixed together and incubate for 15 min. Multiplex PCR was HPRT probe (10 μM), 0.4 μL). HPRT Primer used as (F+R, 20 μM each), 0.2 μL (final concentration is 200 nM). PCS probe was (10 μM, 0.4 μL). PCS Primer (F+R, 20 uM each), 0.8 ul (final concentration is 800 nM). TaqPath 2× master mix was used as 10 μL.

FIG. 21. Knockdown efficacy study of Modified PCSK9 siRNA. HepG2: 2×105 cells per well in 12 well plate in transfection time for 24 hr. cDNA was 30 ng in 8.2 4. PCS siRNA concentrations in tranfection: 10 nM, 1 nM, 0.1 nM, and 0.01 nM, 10× serial dilution. Lipofectamine RNAi Max 2 μL/Rxn in 98 ul OptiMEM; siRNA was applied in 100 μL of OptiMEM; then mixed together and incubate for 15 min. Multiplex PCR was HPRT probe (10 μM), 0.4 μL ). HPRT Primer used as (F+R, 20 μM each), 0.2 viL (final concentration is 200 nM). PCS probe was (10 μM, 0.4 μL ). PCS Primer (F+R, 20 μM each), 0.8 μL (final concentration is 800 nM). TaqPath 2× master mix was used as 10 μL.

FIG. 22. Knockdown efficacy study of Modified PCSK9 siRNA. HepG2: 2×105 cells per well in 12 well plate in transfection time for 24 hr. cDNA was 30 ng in 8.2 μL. PCSK9 siRNA concentrations in tranfection: 10 nM, 1 nM, 0.1 nM, and 0.01 nM, 10× serial dilution. Lipofectamine RNAi Max 2 μL/Rxn in 98 μL OptiMEM; siRNA was applied in 100 μL of OptiMEM; then mixed together and incubate for 15 min. Multiplex PCR was HPRT probe (10 μM), 0.4 μL ). HPRT Primer used as (F+R, 20 μM each), 0.2 μL (final concentration is 200 nM). PCS probe was (10 uM, 0.4 μL ). PCS Primer (F+R, 20 uM each), 0.8 μL (final concentration is 800 nM). TaqPath 2× master mix was used as 10 μL.

FIG. 23 (a) and (b) show the siRNA sequences and chemical modified siRNA sequences and the designed siRNA sequences with minimized seed-dependent off-target effects. FIG. 23 (a): Table 1 (SEQ ID NOS 13-72, respectively, in order of appearance by column); and FIG. 23 (b): Table 2 (SEQ ID NOS 73-102, respectively, in order of appearance by column).

FIG. 24 shows the sequence of NM_174936.4 Homo sapiens proprotein convertase subtilisin/kexin type 9 (PCSK9), transcript variant 1. FIG. 24 discloses SEQ ID NO: 103.

FIG. 25 shows the sample identification information for primary mouse or primary human conjugates and controls used in in vitro evaluation.

FIG. 26 shows a table of the structures and siRNA sequences information for PDOv-PSCK9 conjugates used in in vitro evaluation. FIG. 26 discloses (in column 1) SEQ ID NOS 104-119, and (in column 2, in order of appearance) SEQ ID Nos. 120, 120, 120, 28, 121-124, 28, 27, 28, 27, 28, 121, 125, and 124.

FIG. 27 shows dosage curves of PCSK9 compounds (PG04, PG05, PG06 and PG08, each at 7 concentrations between 0.064 nM and 1000 nM) in primary mouse hepatocytes in vitro, using the mouse PSCK9 probe 3610.

FIG. 28 shows the results of in vitro evaluation of PCSK9 compounds (PG02, PG03, PG07, each at 7 concentrations between 0.064 nM and 1000 nM) in primary human hepatocytes, here, using the human PCSK9 probe 5399.

FIG. 29 shows the results of in vitro evaluation of PCSK9 compounds (PG02, PG03, PG07, each at 10 concentrations ranging from 0.001024 nM through 2000 nM) in primary human hepatocytes using human PCSK9 probe 5399.

FIG. 30 shows results of in vivo evaluation of a single dose of the PCSK9 compound PG05 at 1, 3 and 10 mg/kg on Day 14 (sacrifice) in liver tissue samples of C_{57/}Black6 mice using qPCR.

FIG. 31 shows the results of in vivo evaluation of PCSK9 compound PG05 at 1, 3 and 10 mg/kg on Day 14 (sacrifice) in blood samples of C_{57/}Black6 using ELISA.

FIG. 32 shows the lack of toxic effect of PBS or 1, 3 or 10 mg/kg of PG05 on body weight of mice from date of arrival through sacrifice at Day 14.

FIG. 33 shows the in vivo evaluation of the effect of PCSK9 conjugate PG04 versus saline alone to reduce the expression of PCSK9 in plasma and in liver lysates of C_{57/}B16 mice using ELISA.

FIG. 34 shows serum transaminase (ALT, AST) levels in mice administered PG04 and saline prior to the start of the study and at Days 1, 3, 7 and 14.

FIG. 35 is a table showing the sequence/structure of PCSK9 duplexes PG13 and PC, the latter, Alnylam's GalNAc-PCSK9 conjugate with the same sequence as PG13, used for evaluation in the vivo comparison study in a hyperlipidemia mouse model). FIG. 35 discloses SEQ ID NOS 126-128, and 128, respectively, in order of appearance by column.

FIG. 36 (a), (b) and (c) show the design and results of a study in a hyperlipidemic mouse model comparing the effect of GalNAc-PCSK9 conjugates in vivo. FIG. 36 (a) shows the basic study design, with the mice in each of four groups (n=8) receiving a single dose of saline (control group), PC (3 mg/kg of Alnylam's GalNAc/PCSK9 conjugate), or low (1 mg/kg) or high (3 mg/kg) doses of STP135G, Sirnaomics' GalNAc-PDoV/PCSK9 conjugate; FIG. 36 (b) shows the mean LDLc plasma levels following administration of a single subcutaneous injection at Days 7 and 14; and FIG. 36 (c) shows the mean PCSK9 plasma levels (pg/mL) following administration of a single subcutaneous injection at Day 14.

### DETAILED DESCRIPTION

Compositions and methods using interfering RNA (RNAi) molecules that inhibit the expression of PCSK9 gene in a cell, such as a cell within a subject, and having enhanced therapeutic benefit are provided. The compositions and methods allow targeted cell/tissue delivery of a therapeutic compound, such as an siRNA molecule, to a subject by linking a targeting ligand to the compound. The subject may be an animal or a human.

### Definitions

As used herein, “oligonucleotide” refers to a chemically modified or unmodified nucleic acid molecule (RNA or DNA) having a length of less than 100 nucleotides (for example less than50, less than 30, or less than 25 nucleotides). It can be siRNA, microRNA, anti microRNA, microRNA mimics, dsRNA, ssRNA, aptamer, triplex forming oligonucleotides, aptamers. In one embodiment, the oligonucleotide is an RNAi agent. The term “ribonucleotide” or “nucleotide” or “deoxyribonucleotide” can also refer to a modified nucleotide, as further detailed below, or a surrogate replacement moiety. The person of ordinary skill in the art is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the disclosed embodiments by a nucleotide containing, for example, inosine. Sequences comprising such replacement moieties are included in the disclosed embodiments.

The term “antisense strand” refers to the strand of a double stranded RNAi agent which includes a region that is substantially complementary to a target sequence (e.g., a human PCSK9 mRNA). As used herein, the term “region complementary to part of an mRNA encoding transthyretin” refers to a region on the antisense strand that is substantially complementary to part of a PCSK9 mRNA sequence. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the person of ordinary skill in the art. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. For example, a complementary sequence is sufficient to allow the relevant function of the nucleic acid to proceed, e.g., RNAi. The person of ordinary skill in the art will be able to determine the set of conditions most appropriate for a test of complementarity of two sequences in accordance with the ultimate application of the hybridized nucleotides.

Sequences can be “fully complementary” with respect to each when there is base-pairing of the nucleotides of the first nucleotide sequence with the nucleotides of the second nucleotide sequence over the entire length of the first and second nucleotide sequences. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogstein base pairing.

The terms “complementary,” “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding PCSK9) including a 5′ UTR, an open reading frame (ORF), or a 3′ UTR. For example, a polynucleotide is complementary to at least a part of a PCSK9 mRNA if the sequence is substantially complementary to a non-interrupted portion of an mRNA encoding PCSK9.

The term “inhibiting,” as used herein, is used interchangeably with “reducing,” “silencing,” “downregulating,” “suppressing” and other similar terms, and includes any level of inhibition.

Inhibiting expression of a PCSK9 includes inhibition of expression of any PCSK9 gene (such as, e.g., a mouse PCSK9 gene, a rat PCSK9 gene, a monkey PCSK9 gene, or a human PCSK9 gene) as well as variants, (e.g., naturally occurring variants), or mutants of a PCSK9 gene. Thus, the PCSK9 gene may be a wild-type PCSK9 gene, a mutant PCSK9 gene, or a transgenic PCSK9 gene in the context of a genetically manipulated cell, group of cells, or organism.

Inhibiting expression of a PCSK9 gene includes any level of inhibition of a PCSK9 gene, e.g., at least partial suppression of the expression of a PCSK9 gene, such as an inhibition of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%. at least about 95%, at least about 96%, at least about 97%, at least about 98%, or at least about 99%.

The expression of a PCSK9 gene may be assessed based on the level of any variable associated with PCSK9 gene expression, e.g., PCSK9 mRNA level, PCSK9 protein level, or serum lipid levels. Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control)

As used herein, an “siRNA molecule” or “RNAi molecule” is a duplex oligonucleotide,

hat is a short, double-stranded polynucleotide, that interferes with the expression of a gene in a cell, after the molecule is introduced into the cell. For example, an siRNA molecule targets and binds to a complementary nucleotide sequence in a single stranded target RNA molecule. By convention, when an siRNA molecule is identified by a particular nucleotide sequence, the sequence refers to the sense strand of the duplex molecule. One or more of the ribonucleotides comprising the molecule can be chemically modified by techniques known in the art. In addition to being modified at the level of one or more of its individual nucleotides, the backbone of the oligonucleotide can be modified. Additional modifications include the use of small molecules (e.g. sugar molecules), amino acids, peptides, cholesterol, and other large molecules for conjugation onto the siRNA molecule. The terms “iRNA”, “RNAi agent,” “iRNA agent,”, “RNA interference agent” may also be used interchangeably herein with siRNA molecular or RNAi molecule, and refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of PCSK9 in a cell, e.g., a cell within a subject, such as a mammalian subject.

In one embodiment, an RNAi agent in the disclosed embodiments include a single stranded RNA that interacts with a target RNA sequence, e.g., a PCSK9 target mRNA sequence, to direct the cleavage of the target RNA. Without wishing to be bound by theory, it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, one embodiment relates to a single stranded RNA (siRNA) generated within a cell and which promotes the formation of a RISC complex to effect silencing of the target gene, i.e., a PCSK9 gene.

Double-stranded RNA has been shown to silence gene expression via RNA interference (RNAi). Short-interfering RNA (siRNA)-induced RNAi regulation shows great potential to treat a wide variety of human diseases from cancer to other traditional undruggable disease, but problems remain with delivery of the siRNA to the desired tissue. In particular, improved targeting of nucleic acid drugs to specific cell types or tissues is needed, together with development of non-toxic endosomal escape agents, as explained further.

A PCSK9-associated disease is intended to include any disease associated with the PCSK9 gene or protein. Such a disease may be caused, for example, by excess production of the PCSK9 protein, by PCSK9 gene mutations, by abnormal cleavage of the PCSK9 protein, by abnormal interactions between PCSK9 and other proteins or other endogenous or exogenous substances. Exemplary PCSK9-associated diseases include lipidemias, e.g., a hyperlipidemias, and other forms of lipid imbalance such as hypercholesterolemia, hypertriglyceridemia and the pathological conditions associated with these disorders such as heart and circulatory diseases.

“Therapeutically effective amount,” as used herein, is intended to include the amount of an RNAi agent that, when administered to a patient for treating a PCSK9 associated disease, is sufficient to effect treatment of the disease (e.g., by diminishing, ameliorating or maintaining the existing disease or one or more symptoms of disease). The “therapeutically effective amount” may vary depending on the RNAi agent, how the agent is administered, the disease and its severity and the history, age, weight, family history, genetic makeup, stage of pathological processes mediated by PCSK9 expression, the types of preceding or concomitant treatments, if any, and other individual characteristics of the patient to be treated.

“Prophylactically effective amount,” as used herein, is intended to include the amount of an RNAi agent that, when administered to a subject who does not yet experience or display symptoms of a PCSK9-associated disease, but who may be predisposed to the disease, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an RNAi agent that produces some desired local or systemic effect at a reasonable benefit/risk ratio applicable to any treatment. RNAi agents employed in the methods of the present disclosed embodiments may be administered in a sufficient amount to produce a reasonable benefit/risk ratio applicable to such treatment.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In preferred embodiments, a “sample derived from a subject” refers to blood or plasma drawn from the subject. In further embodiments, a “sample derived from a subject” refers to liver tissue (or subcomponents thereof) derived from the subject

In particular, improved targeting of nucleic acid drugs to specific cell types or tissues is needed, together with development of non-toxic endosomal escape agents, as explained furtherbelow.

Currently, two type of effective delivery methods for nucleic acid drugs have been used. One method uses lipid-based nanoparticle (liposomes), that contain multiple components. The other method targets the asialoglycoproteins receptor (“ASGPR”) using conjugates that contain the GalNAc molecule.

A major challenge for RNA-based therapeutics is that all pathways for delivery to cells eventually lead to endosomal escape. ASO and siRNA delivery to the liver can be achieved using ASGPR-targeted GalNAc-siRNA conjugates due to the properties of ASGPR that are well suited for macromolecular drug deliver to hepatocytes. In particular, hepatocytes express millions of copies of ASGPR on their cell surface, which cycle at a rapid rate of every 10-15 min. These properties make a GalNAc-based delivery approach effective even with a presumed endosomal escape rate of <0.01%. By contrast, effective delivery of ASO or RNA to other tissues has not been achieved. No other ligand—receptor system expresses receptors at such a high level as ASGPR,nor cycles into endosomes as rapidly. Indeed, most cell surface receptors are expressed in the range of 10,000-100,000 per cell (or lower), and caveolin and clathrin-mediated endocytosis typically recycles every 90 min. See Juliano, Nucleic Acids Res. 44, 6518-6548 (2016).

Endosomal escape remains a problem that applies to all RNA-based therapeutics. Enhancing endosomal escape by developing new chemistries and materials is needed to target the cell or tissue beyond the liver hepatocytes. Small-molecule endosomolytic agents such as chloroquine have been used to disrupt or lyse endosomes, but at the effective concentration these agents invariably lyse all types of endosomes inside the cell resulting in substantial toxicity.

An alternative endosomal escape approach is to conjugate endosomolytic peptides or molecules directly to the RNA, which will strictly limit their action to endosomes containing the RNA therapeutic. Various clinical trials using a two-molecule dynamic polyconjugate (DPC) system containing cholesterol or lytic melittin peptide to escape the endosome were terminated due to toxicity effects. Wooddell, et al., Mol. Ther. 21, 973-985 (2013); Hou et al., Biotechnol. Adv. 33, 931-940 (2015).

### Peptide Docking Vehicle

“Peptide Docking Vehicle” (PDoV) refers to a synthetic peptide of defined sequence that contains multiple conjugation sites to allow conjugation with one or more targeting ligands and with one or more oligonucleotides. It contains functional groups, such as a hydrophobic chain or a pH sensitive residue, which facilitate the release of the oligonucleotide payload entrappedinside of the endosome of a cell after delivery of the conjugated PDoV to the cell.

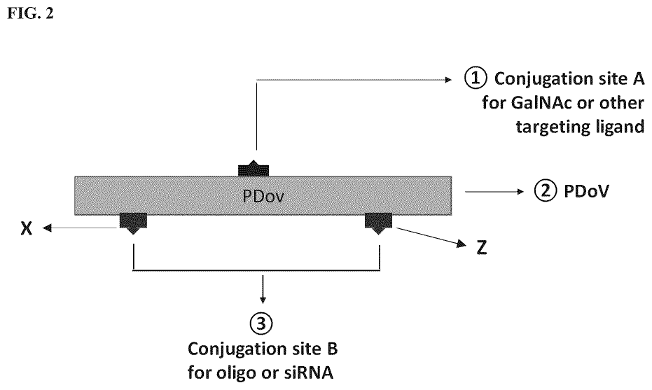

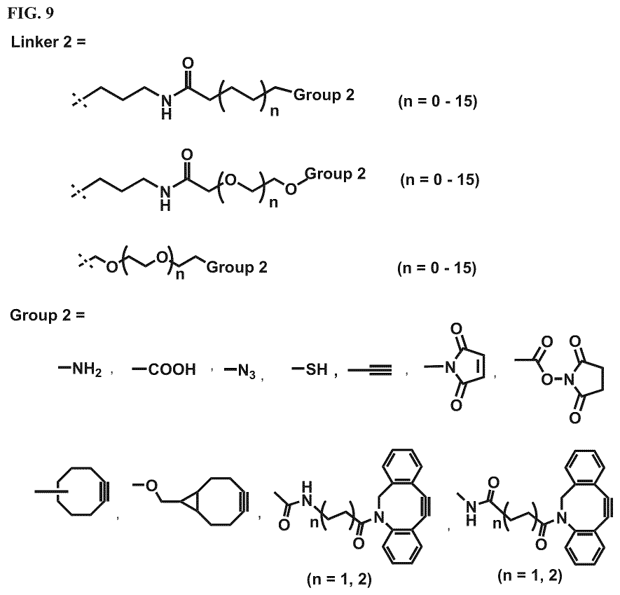

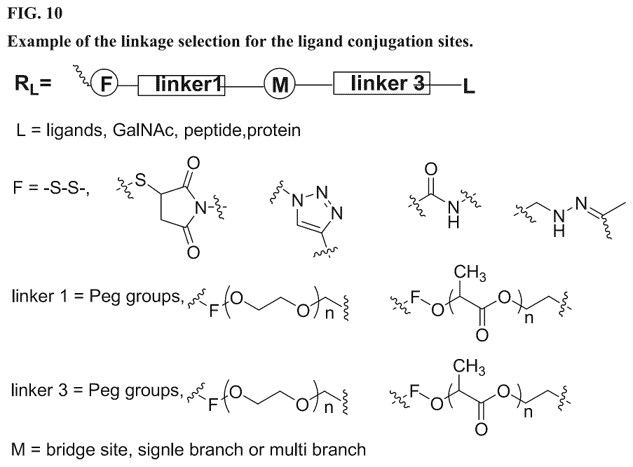

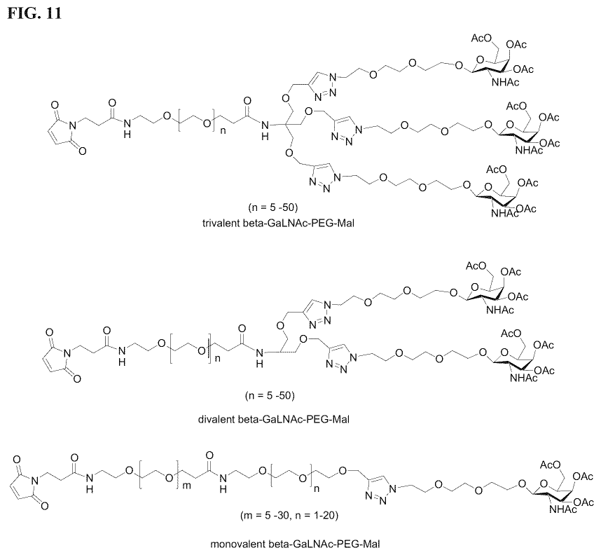

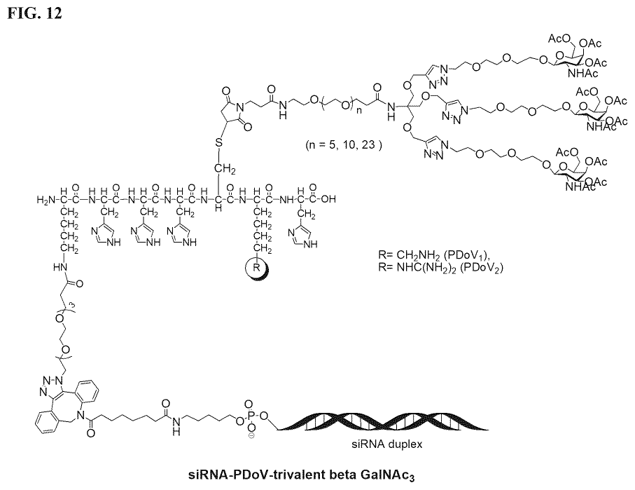

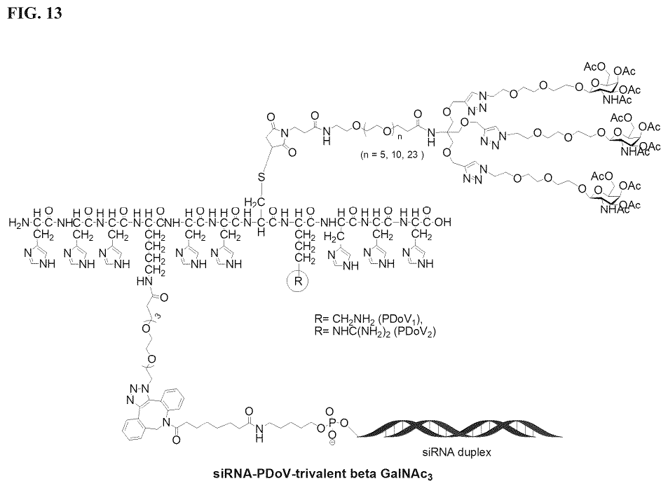

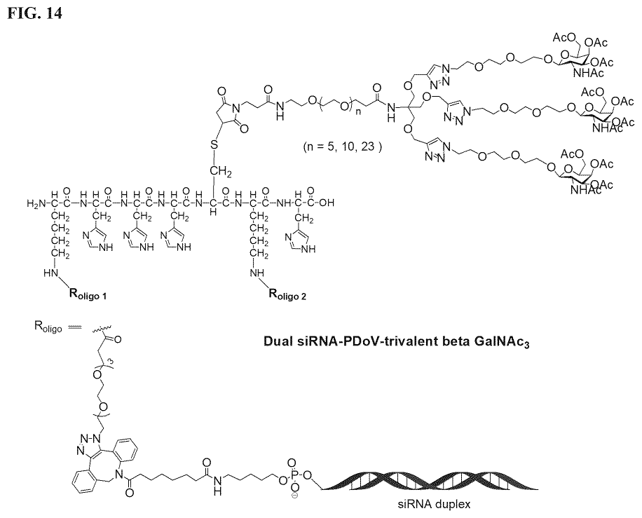

The Peptide Docking Vehicle (PDoV) advantageously has one ligand conjugation site together with multiple oligonucleotide sites. The PDoV has a peptide backbone with the general structure: (HₙKₘ)ₒXₚZ_{q} (SEQ ID NO: 3) with multiple repeating units of histidine (H), lysine (K) and functional units X and Z (where X or Z is an amino acid, or an amino acid derivative [can be selected from Linker 1 and functional group in FIG. 9], and where: n=1-10; m=1-10; o=1-10, p=1-5, and q=1-5. HK repeating units have been demonstrated to facilitate endosome release. The lysine residues or the functional unit(s) X may be used as docking sites for the conjugation of ligands and Y provides docking sites for the conjugation of oligonucleotide via a different covalent linkage. The diagram in FIG. 2 shows a schematic of how the PDoV may be conjugated. For example, site \textcircled{1} is only able to react in the presence of ligand such as GalNAc or other targetingligands. Site \textcircled{3} can only conjugate with oligonucleotide and siRNA under selected conditions. (see FIG. 10 for the F: functional conjugation method].

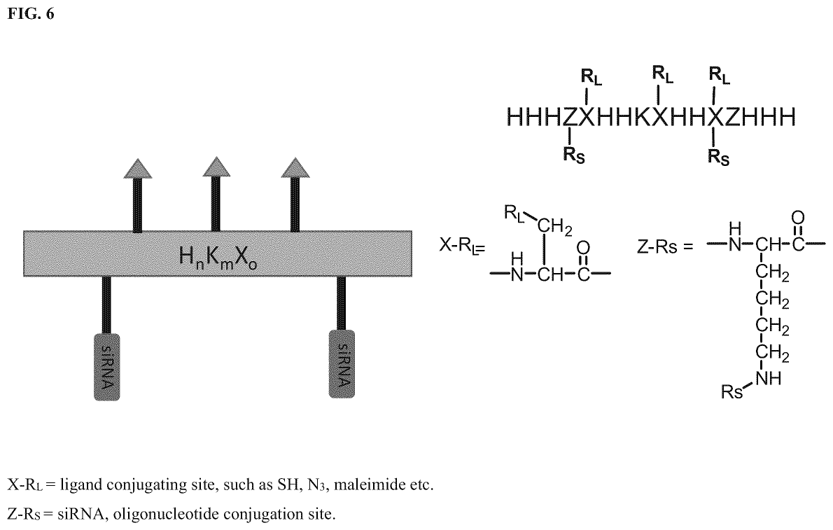

Alternatively, the PDoV may have three ligand conjugation sites and multi oligonucleotide sites (see FIG. 6): a (HₙKₘ)ₒXₚZ_{q} (SEQ ID NO: 3) peptide back bone has multi-repeating units of histidine (H), lysine (K) and functional units X and Z (amino acid, or functional linker), where the n=1-10, m=1-10, o=1-10, p=1-5, q=1-5. HK repeating units have been demonstrated to have good cell penetrating ability and to facilitate endosome release. The lysine or the various functional units X are adapted as the docking sites for the conjugation of ligands, and Z is adapted the docking sites for the conjugation of oligonucleotides through different covalent linkages.

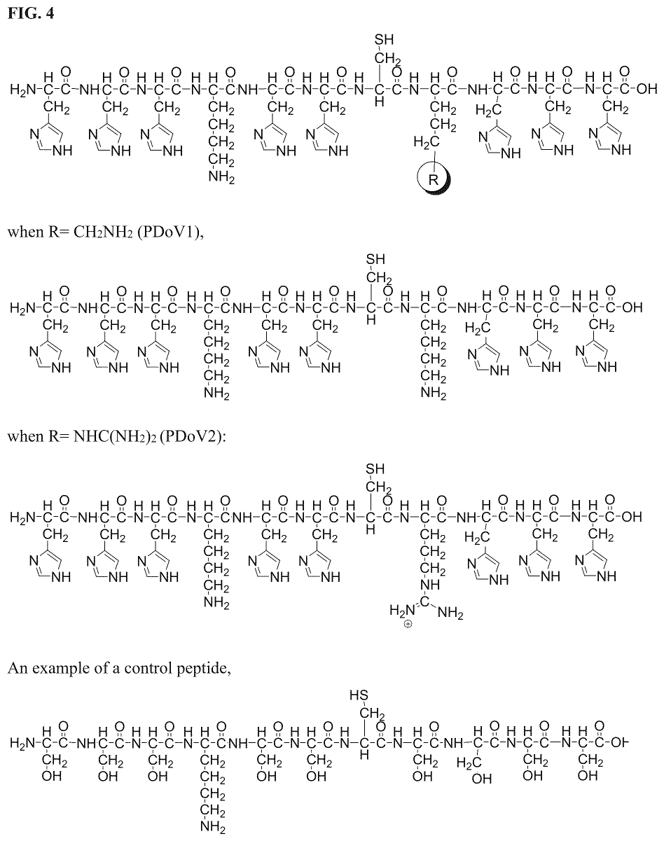

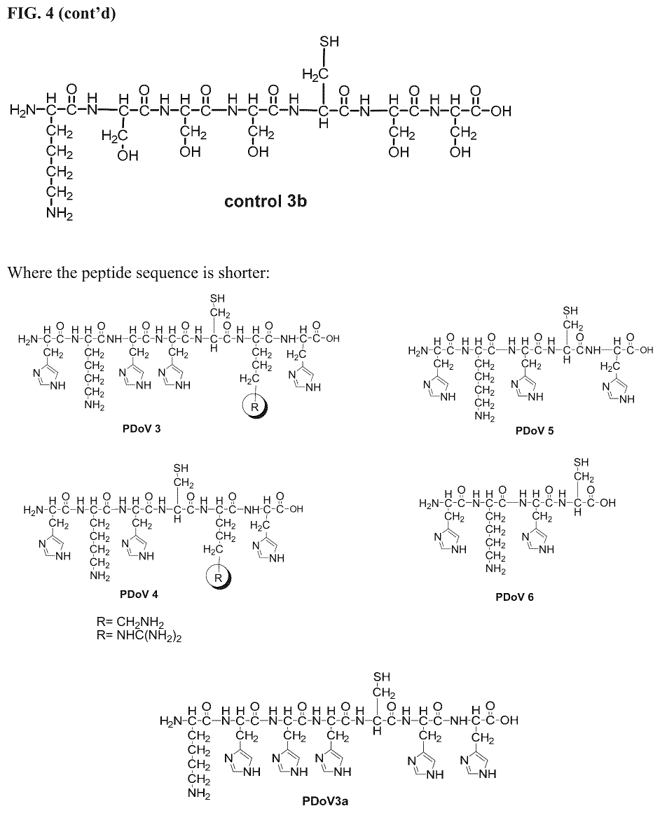

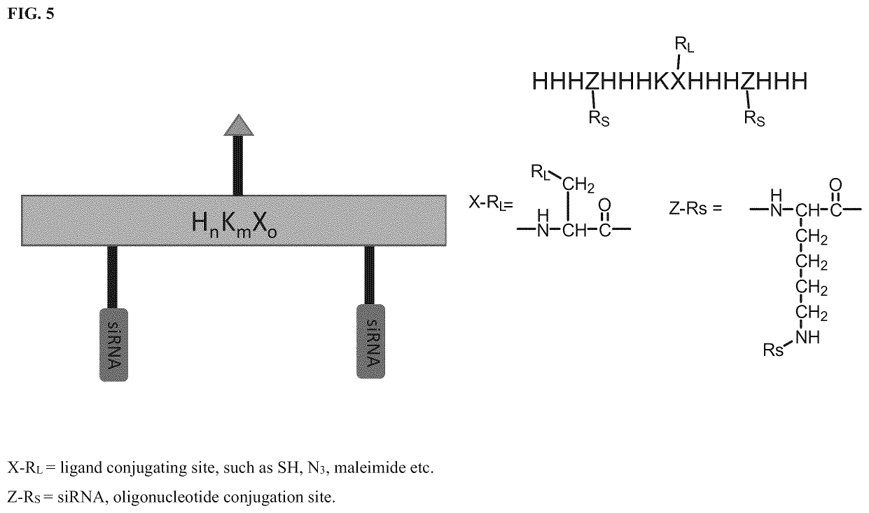

In the structure design, the PDoV construct is an endosome releasing peptide inserted with multiple conjugation sites X and Z. Site X is used to conjugate the targeting ligand, and siteZ is used to conjugate multiple oligonucleotide or nucleic acid. Some examples of the constructs for the PDoV are shown in FIG. 4, where: A represents peptide sequence K, R, H, HH, HUH, HHHH (SEQ ID NO: 1), HHK, HHHK (SEQ ID NO: 2) or other short peptide; B represents peptide sequence K, R, H, HH, HHH, HHHH (SEQ ID NO: 1), HHK, HHHK (SEQ ID NO: 2), or other short peptide or other amino acid or combination; D represents oligonucleotide, siRNA, mRNA, or aptamer; RL represents ligand; and RS represents a linker to the oligonucleotide. In some embodiments, the peptide contains 5-15 amino acids.

In some embodiment, the PDoV has a structure as shown in FIG. 4.

Compositions and methods using interfering RNA molecules having enhanced therapeutic benefit are provided. The compositions and methods allow targeted cell/tissue delivery of a therapeutic compound, such as an siRNA molecule, to a subject by linking a targeting ligand to the compound. The subject may be an animal or a human.

Ligands can improve transport, hybridization, and specificity properties and may also improve nuclease resistance of the resultant natural or modified oligoribonucleotide, or a polymeric molecule comprising any combination of monomers described herein and/or natural or modified ribonucleotides.

Ligands in general can include therapeutic modifiers, e.g., for enhancing uptake; diagnostic compounds or reporter groups e.g., for monitoring distribution; cross-linking agents; and nuclease-resistance conferring moieties. General examples include lipids, steroids, vitamins, sugars, proteins, peptides, polyamines, and peptide mimics.

Target ligands can include a naturally occurring substance, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g., an aptamer). Examples of polyamino acids include polyamino acid is a polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Other examples of target ligands include dyes, intercalating agents (e.g., acridines), cross-linkers (e.g., psoralene, mitomycin C), porphyrins (TPPC_{4,} texaphyrin, Sapphyrin), polycyclic aromatic hydrocarbons (e.g., phenazine, dihydrophenazine), artificial endonucleases or a chelator (e.g., EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, multivalent fucose, or aptamers. The ligand can be, for example, a lipopolysaccharide, an activator of p38 MAP kinase, or an activator of NF-.kappa.B.

The target ligand can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

The target ligand can increase the uptake of the oligonucleotide into the cell by, for example, activating an inflammatory response. Exemplary ligands that would have such an effect include tumor necrosis factor alpha (TNFalpha), interleukin-1 beta, or gamma interferon.

In one aspect, the target ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based target ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

Peptide and peptidomimetic target ligands include those having naturally occurring or modified peptides, e.g., D or L peptides; .alpha., .beta., or .gamma. peptides; N-methyl peptides; azapeptides; peptides having one or more amide, i.e., peptide, linkages replaced with one or more urea, thiourea, carbamate, or sulfonyl urea linkages; or cyclic peptides.

The targeting ligand can be any ligand that is capable of targeting a specific receptor. Examples are: folate, GalNAc, galactose, mannose, mannose-6P, clusters of sugars such as GalNAc cluster, mannose cluster, galactose cluster, or an aptamer. A cluster is a combination of two or more sugar units. The targeting ligands also include integrin receptor ligands, Chemokine receptor ligands, transferrin, biotin, serotonin receptor ligands, PSMA, endothelin, GCPII, somatostatin, LDL and HDL ligands. The ligands can also be based on nucleic acid, e.g., an aptamer. The aptamer can be unmodified or have any combination of modifications disclosed herein

In some embodiments, the targeting ligand as described herein may be conjugated to an endosome releasing peptide through an orthogonal bioconjugation method. The targeting ligand may particularly be used to improve the delivery of RNAi molecules to a selected target, such as the liver. In other embodiments, the targeting ligand(s) permit targeted delivery of RNAi molecules into other tissues, for example, in the skin and brain.

The targeting ligands as described herein may include one or more targeting moieties, one or more linkers. The linkers covalently conjugated with the siRNA and targeting ligands through click chemistry, thiol/maleimide chemistry, or other bioorthogonal chemistry. Linkers advantageously are hydrophilic and can be, for example, a water soluble flexible polyethylene glycol (PEG) which is sufficiently stable and limits the potential interaction between one or more targeting moiety(s). PEG has been validated to be safe and compatible for therapeutic purposes from clinical studies. In some embodiments, the linker can be poly(L-lactide)n (where n=5-20), where the ester bond is enzymatically or hydrolytically labile.

The targeting ligand may include one or more targeting moieties, one or more groups with a linker reactive connection moiety. They are covalently conjugated with the siRNA and targeting ligands through click chemistry, thiol/maleimide chemistry, or other bioorthogonal chemistry. The linker reactive connection moiety may be, but is not limited to, a thiol-maleimide linkage, a triazol linkage formed by reaction of an alkyne and an azide, and an amide formed from an amine-NHS ester linkage. Each of these linkages is suitable for covalently linking both the targeting ligands and the therapeutic compound.

In some embodiments, the targeting ligands disclosed here include one or more targeting moieties, one or more linkers with reactive connection moiety. The linker contains a thiol moiety, or maleimide moiety, carboxylic acid, or amine, azido group, alkyne group, and the like.

In some embodiments, the targeting specific RNA compound disclosed herein can be directly conjugated to an endosome releasing docking peptide via the 3′ or 5′ terminal end of the RNA. The targeting ligand (for example N-acetyl-galactosamine) also be conjugated with the same docking peptide in a compatible method.

In some embodiments, the targeting specific RNA compound disclosed herein can also be directly conjugated to a targeting ligand (for example N-acetyl-galactosamine), via, for example, the 3′ or 5′ terminal end of the RNA. In some embodiments, the RNA may contain one or more modified nucleotides such as 3′-OMe, 3′-F, or 3′-MOE. In some embodiments, the RNA can be an RNAi agent, for example a double stranded RNAi agent. In some embodiments, the targeting ligands disclosed herein are linked to the 5′ or 3′ terminus of the sense strand of a double stranded RNAi agent or the 5′ or 3′ terminus of the antisense strand of a double stranded RNAi agent. The targeting ligands may alternatively be linked to both 3′/3″, 3′/5′ or 5′/5′ terminal end of the sense and antisense strand of a double stranded RNAi agent.

The targeting ligands may be covalently bonded to the RNAi molecule via, for example, a phosphate, phosphorothioate, or phosphonate group at the 3′ or 5′ terminus of the sense strand of a double stranded RNAi agent. In some embodiments, the targeting specific RNA compound disclosed herein is a PCSK9, mRNA expression-inhibition specific compound.

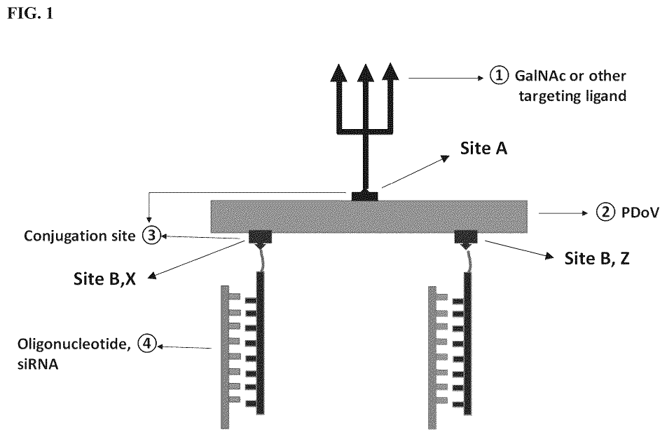

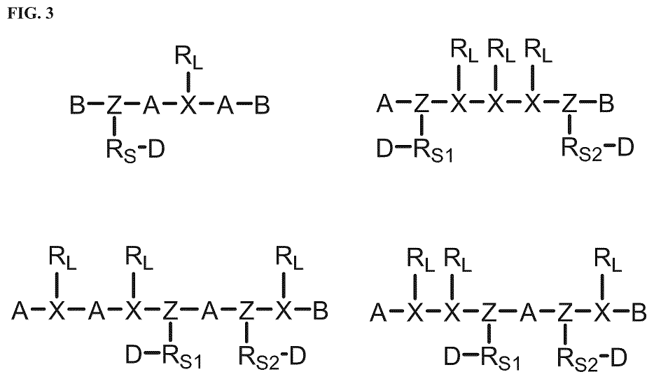

The PDoV enhances escape of its macromolecular cargo into the cellular cytoplasm in a non-toxic manner. This allows effective delivery of, for example, RNAi therapeutics. An endosomal escape peptide (PDoV)is provided that enhances escape of macromolecular cargo, such as an siRNA molecule, into the cytoplasm in a non-toxic manner. Various examples of the PDoV platform are shown in FIGS. 1-4. In the PDoV the endosomal escaping peptide acts both as the docking site linker for the RNA and the targeting ligands. Multiple RNA molecules can be conjugated with the same construct to achieve codelivery of siRNA molecules against different target mRNAs, thereby providing a synergistic benefit for silencing a multi-disease related gene. The histidine and lysine rich polypeptide or linear histidine and lysine rich peptide has been shown to be an effective cell penetrating and endosomal release agent in the delivery of RNA. The peptide contains a histidine rich domain, where the imidazole rings of the histidine residues are protonated at a lower pH value (pH<˜6) and act inside the endosome as a proton sponge, which leads to lysis of the endosome lipid bilayers and release of the RNA. The conjugation sites on the PDoV are described in more detail below.

In the next few sections, each component will be discussed in detail including RNAi agent, targeting ligands, linkers between RNAi and peptide, linkers between ligand and peptide, and endosome releasing docking peptide.

### RNAi Agents

The RNAi molecules are double stranded compounds. For example, the double stranded siRNA can be anti PCSK9, and can be unmodified or chemically modified at the 2′ position with, for example, 2′-OCH₃, 2′-F, or 2′-O-MOE, or at the 5′ position with —P(O)₂═S. Other chemical modifications are known in the art and can include, for example, pegylation or lipid functionalization to improve the overall stability and bioavailability of the RNAi.

In specific embodiments, the double stranded siRNA may be duplexes consisting of 24, 23, 22, 21, 20, 19, 18, 17 or 16 contiguous base pairs of any one or more of the duplexes in Table 1 and Table 2. In other embodiments, the siRNA molecule contains a duplex of two complimentary, single-stranded oligonucleotides that have the same length and where each oligonucleotide has a length of 10-29 bases or 19-27 bases. The duplexes may be blunt-ended, or may have 1 or 2 base overhangs at the duplex termini.

As nucleic acids are polymers of subunits, many of the modifications occur at a position which is repeated within a nucleic acid, e.g., a modification of a base, or a phosphate moiety, or a non-linking O of a phosphate moiety. In some cases the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of a RNA or may only occur in a single strand region of a RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. For example, it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

The RNAi agent may further comprise at least one phosphorothioate or methylphosphonate internucleotide linkage. The phosphorothioate or methylphosphonate internucleotide linkage modification may occur on any nucleotide of the sense strand or antisense strand or both strands in any position of the strand. For instance, the internucleotide linkage modification may occur on every nucleotide on the sense strand and/or antisense strand; each internucleotide linkage modification may occur in an alternating pattern on the sense strand and/or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand

### Targeting Ligands

The targeting ligand moiety may be, for example, N-acetyl-galactosamine (GalNAc), galactose, galactosamine, N-formal-galactosamine, N-propionyl-galactosamine, N-butanoylgalactosamine, cRGD, GLP peptide or other small molecules. The targeting ligands are covalently coupled to the peptide by a covalent bond. The number ligands can be 1, 2, or 3. The targeting ligands disclosed here were has a structure represented by the following:

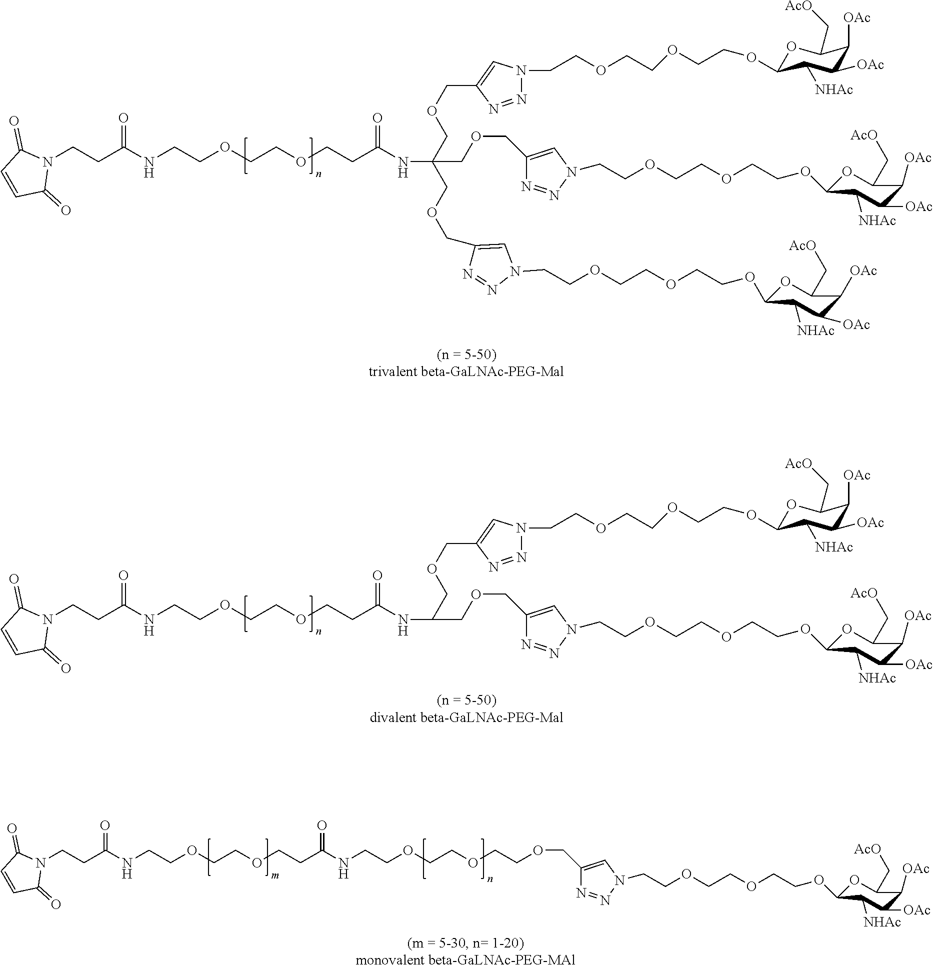

Linkers between RNAi and Peptide

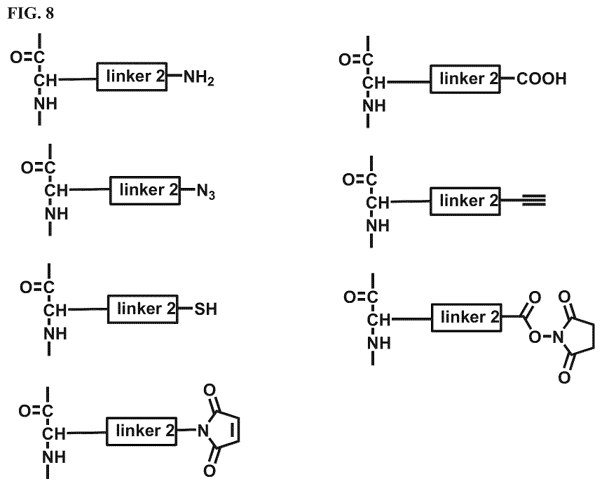

Linkage for the ligand conjugation Rs: Chemical group Rs may be one of various “click” like reactive moieties used to conjugate the oligonucleotide with the PDoV peptide vehicle. Rs can be amine, hydrazine, N-hydroxysuccinimide, azido, alkyne, carboxylic acid, thiol, or maleimide, or other chemical reactive moieties known in the art. Representative examples are shown in FIGS. 8 and 9:

The linker 2 in the conjugation Rs-linker2-siRNA is a chemical spacer disposed between the peptide and the conjugation site, which allow the conjugation site to be attached at the linker's terminal site. The linker 2 can be an aliphatic chain or a polyethylene glycol chain, or other hydrophobic lipid or hydrophilic chain. The group 2 at the end site is the reactive site for the chemical conjugation with the siRNA end.

Linkers between Ligand and Peptide

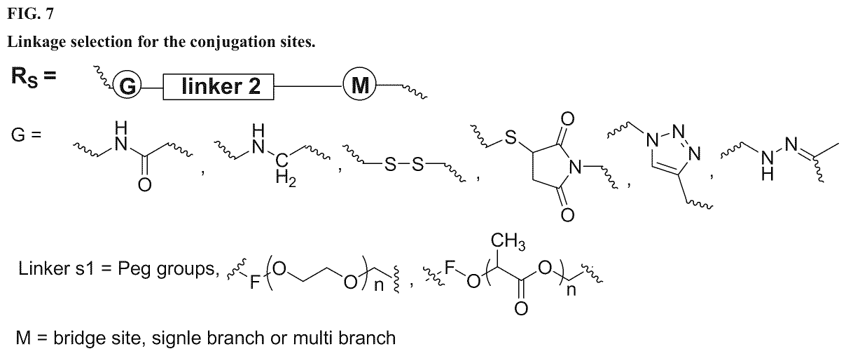

The targeting ligand and the RNAi moiety disclosed herein contains a linker-1, which directly connect the siRNA (3′ or 5′ end of the sense strand) and the bridge that connects the linker 2-ligands (FIG. 7). The spacing of the linker-1 is a linear polyethylene glycol, wherein the number of ethylene glycol units is 1 to 50, or poly(L-lactide) wherein the number of repeating units of ethyl ester is between 1 to 50 or average molecular weight from 100 to 3500. The conjugation site can be a maleimide/thiol group or selected from the Group 2 list in FIG. 9.

### Pharmaceutical Compositions

The present embodiments include pharmaceutical compositions and formulations, which include the iRNAs. In one embodiment, provided herein are pharmaceutical compositions containing an iRNA, as described herein, and a pharmaceutically acceptable carrier. The pharmaceutical compositions containing the iRNA are useful for treating a disease or disorder associated with the expression or activity of a PCSK9 gene, e.g. a lipid disorder. Such pharmaceutical compositions are formulated based on the mode of delivery. One example is compositions that are formulated for systemic administration via parenteral delivery, e.g., by intravenous (IV) delivery. Another example is compositions that are formulated for direct delivery into the brain parenchyma, e.g., by infusion into the brain, such as by continuous pump infusion.

The pharmaceutical compositions comprising RNAi agents of the disclosed embodiments may be, for example, solutions with or without a buffer, or compositions containing pharmaceutically acceptable carriers. Such compositions include, for example, aqueous or crystalline compositions, liposomal formulations, micellar formulations, emulsions, and gene therapy vectors.

In the methods of the disclosed embodiments, the RNAi agent may be administered in a solution. A free RNAi agent may be administered in an unbuffered solution, e.g., in saline or in water. Alternatively, the free siRNA may also be administered in a suitable buffer solution. The buffer solution may comprise acetate, citrate, prolamine, carbonate, or phosphate, or any combination thereof. In a preferred embodiment, the buffer solution is phosphate buffered saline (PBS). The pH and osmolarity of the buffer solution containing the RNAi agent can be adjusted such that it is suitable for administering to a subject.

In some embodiments, the buffer solution further comprises an agent for controlling the osmolarity of the solution, such that the osmolarity is kept at a desired value, e.g., at the physiologic values of the human plasma. Solutes which can be added to the buffer solution to control the osmolarity include, but are not limited to, proteins, peptides, amino acids, non-metabolized polymers, vitamins, ions, sugars, metabolites, organic acids, lipids, or salts. In some embodiments, the agent for controlling the osmolarity of the solution is a salt. In certain embodiments, the agent for controlling the osmolarity of the solution is sodium chloride or potassium chloride.

The pharmaceutical compositions of the disclosed embodiments may be administered in dosages sufficient to inhibit expression of a PCSK9 gene. In general, a suitable dose of an iRNA of the disclosed embodiments will be in the range of about 0.001 to about 200.0 milligrams per kilogram body weight of the recipient per day, generally in the range of about 1 to 50 mg per kilogram body weight per day. For example, the dsRNA can be administered at about 0.01 mg/kg, about 0.05 mg/kg, about 0.5 mg/kg, about 1 mg/kg, about 1.5 mg/kg, about 2 mg/kg, about 3 mg/kg, about 10 mg/kg, about 20 mg/kg, about 30 mg/kg, about 40 mg/kg, or about 50 mg/kg per single dose.

For example, the RNAi agent, e.g., dsRNA, may be administered at a dose of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or about 10 mg/kg. Values and ranges intermediate to the recited values are also intended to be part of the disclosed embodiments.

In other embodiments, the RNAi agent is administered at a dose of about 0.1 to about 50 mg/kg, about 0.25 to about 50 mg/kg, about 0.5 to about 50 mg/kg, about 0.75 to about 50 mg/kg, about 1 to about 50 mg/mg, about 1.5 to about 50 mg/kb, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 20 to about 50 mg/kg, about 25 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.1 to about 45 mg/kg, about 0.25 to about 45 mg/kg, about 0.5 to about 45 mg/kg, about 0.75 to about 45 mg/kg, about 1 to about 45 mg/mg, about 1.5 to about 45 mg/kb, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 20 to about 45 mg/kg, about 25 to about 45 mg/kg, about 25 to about 45 mg/kg, about 30 to about 45 mg/kg, about 35 to about 45 mg/kg, about 40 to about 45 mg/kg, about 0.1 to about 40 mg/kg, about 0.25 to about 40 mg/kg, about 0.5 to about 40 mg/kg, about 0.75 to about 40 mg/kg, about 1 to about 40 mg/mg, about 1.5 to about 40 mg/kb, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 20 to about 40 mg/kg, about 25 to about 40 mg/kg, about 25 to about 40 mg/kg, about 30 to about 40 mg/kg, about 35 to about 40 mg/kg, about 0.1 to about 30 mg/kg, about 0.25 to about 30 mg/kg, about 0.5 to about 30 mg/kg, about 0.75 to about 30 mg/kg, about 1 to about 30 mg/mg, about 1.5 to about 30 mg/kb, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 20 to about 30 mg/kg, about 25 to about 30 mg/kg, about 0.1 to about 20 mg/kg, about 0.25 to about 20 mg/kg, about 0.5 to about 20 mg/kg, about 0.75 to about 20 mg/kg, about 1 to about 20 mg/mg, about 1.5 to about 20 mg/kb, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. Values and ranges intermediate to the recited values are also intended to be part of the disclosed embodiments.

For example, the RNAi agent, e.g., dsRNA, may be administered at a dose of about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or about 10 mg/kg. Values and ranges intermediate to the recited values are also intended to be part of the disclosed embodiments.

In other embodiments, the RNAi agent is administered at a dose of about 0.5 to about 50 mg/kg, about 0.75 to about 50 mg/kg, about 1 to about 50 mg/mg, about 1.5 to about 50 mg/kg, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 20 to about 50 mg/kg, about 25 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.5 to about 45 mg/kg, about 0.75 to about 45 mg/kg, about 1 to about 45 mg/mg, about 1.5 to about 45 mg/kb, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 20 to about 45 mg/kg, about 25 to about 45 mg/kg, about 25 to about 45 mg/kg, about 30 to about 45 mg/kg, about 35 to about 45 mg/kg, about 40 to about 45 mg/kg, about 0.5 to about 40 mg/kg, about 0.75 to about 40 mg/kg, about 1 to about 40 mg/mg, about 1.5 to about 40 mg/kb, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 20 to about 40 mg/kg, about 25 to about 40 mg/kg, about 25 to about 40 mg/kg, about 30 to about 40 mg/kg, about 35 to about 40 mg/kg, about 0.5 to about 30 mg/kg, about 0.75 to about 30 mg/kg, about 1 to about 30 mg/mg, about 1.5 to about 30 mg/kb, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 20 to about 30 mg/kg, about 25 to about 30 mg/kg, about 0.5 to about 20 mg/kg, about 0.75 to about 20 mg/kg, about 1 to about 20 mg/mg, about 1.5 to about 20 mg/kb, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. In one embodiment, the dsRNA is administered at a dose of about 10 mg/kg to about 30 mg/kg. Values and ranges intermediate to the recited values are also intended to be part of the disclosed embodiments.

For example, subjects can be administered a therapeutic amount of iRNA, such as about 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 31, 32, 33, 34, 34, 35, 36, 37, 38, 39, 40, 41, 42, 43, 44, 45, 46, 47, 48, 49, or about 50 mg/kg. Values and ranges intermediate to the recited values are also intended to be part of the disclosed embodiments.

The pharmaceutical composition can be administered once daily, or the iRNA can be administered as two, three, or more sub-doses at appropriate intervals throughout the day or even using continuous infusion or delivery through a controlled release formulation. In that case, the iRNA contained in each sub-dose must be correspondingly smaller in order to achieve the total daily dosage. The dosage unit can also be compounded for delivery over several days, e.g., using a conventional sustained release formulation which provides sustained release of the iRNA over a several day period. Sustained release formulations are well known in the art and are particularly useful for delivery of agents at a particular site, such as could be used with the agents of the present disclosed embodiments. In this embodiment, the dosage unit contains a corresponding multiple of the daily dose.

In other embodiments, a single dose of the pharmaceutical compositions can be long lasting, such that subsequent doses are administered at not more than 3, 4, or 5 day intervals, or at not more than 1, 2, 3, or 4 week intervals. In some embodiments, a single dose of the pharmaceutical compositions of the disclosed embodiments are administered once per week. In other embodiments, a single dose of the pharmaceutical compositions are administered bi-monthly.

The person of ordinary skill in the art will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual isRNAs encompassed by the disclosed embodiments can be made using conventional methodologies or on the basis of in vivo testing using an appropriate animal model, as described elsewhere herein.

Advances in mouse genetics have generated a number of mouse models for the study of various human diseases, such as a bleeding disorder that would benefit from reduction in the expression of PCSK9. Such models can be used for in vivo testing of siRNA, as well as for determining a therapeutically effective dose. Suitable mouse models are known in the art and include, for example, a mouse containing a transgene expressing human PCSK9.

The pharmaceutical compositions of the present disclosed embodiments can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intra-arterial, subcutaneous, intraperitoneal or intramuscular injection or infusion; subdermal, e.g., via an implanted device; or intracranial, e.g., by intra-parenchymal, intrathecal or intraventricular, administration.

The siRNA can be delivered in a manner to target a particular tissue, such as the liver (e.g., the hepatocytes of the liver).

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the siRNAs featured in the disclosed embodiments are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). siRNAs featured in the disclosed embodiments can be encapsulated within liposomes or can form complexes thereto, in particular to cationic liposomes. Alternatively, siRNAs can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof). Topical formulations are described in detail in U.S. Pat. No. 6,747,014.

Pharmaceutical compositions of the disclosed embodiments include, but are not limited to, solutions, emulsions, and liposome-containing formulations. These compositions can be generated from a variety of components that include, but are not limited to, preformed liquids, self-emulsifying solids and self-emulsifying semisolids. Particularly preferred are formulations that target the liver when treating hepatic disorders such as hepatic carcinoma.

The pharmaceutical formulations of the disclosed embodiments, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the disclosed embodiments can be formulated into any of many possible dosage forms such as, but not limited to, tablets, capsules, gel capsules, liquid syrups, soft gels, suppositories, and enemas. The compositions of the disclosed embodiments can also be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

Inhibiting pcsk9 Expression

The disclosed embodiments provides methods of inhibiting expression of a Proprotein Convertase Subtilisin Kexin 9 (PCSK9) in a cell. The methods include contacting a cell with an RNAi agent, e.g., a double stranded RNAi agent, in an amount effective to inhibit expression of the PCSK9 in the cell, thereby inhibiting expression of the PCSK9 in the cell.

Contacting of a cell with a double stranded RNAi agent may be done in vitro or in vivo. Contacting a cell in vivo with the RNAi agent includes contacting a cell or group of cells within a subject, e.g., a human subject, with the RNAi agent. Combinations of in vitro and in vivo methods of contacting are also possible. Contacting may be direct or indirect, as discussed above. Furthermore, contacting a cell may be accomplished via a targeting ligand, including any ligand described herein or known in the art. In preferred embodiments, the targeting ligand is a carbohydrate moiety, e.g., a GalNAc ligand, or any other ligand that directs the RNAi agent to a site of interest.

Inhibiting the expression of a PCSK9 refers to inhibiting of expression of any PCSK9 gene (such as, e.g., a mouse PCSK9 gene, a rat PCSK9 gene, a monkey PCSK9 gene, or a human PCSK9 gene) as well as variants or mutants of a PCSK9 gene. Thus, the PCSK9 gene may be a wild-type PCSK9 gene, a mutant PCSK9 gene, or a transgenic PCSK9 gene in the context of a genetically manipulated cell, group of cells, or organism.

“Inhibiting expression of a PCSK9 gene” includes any level of inhibition of a PCSK9 gene, e.g., at least partial suppression of the expression of a PCSK9 gene. The expression of the PCSK9 gene may be assessed based on the level, or the change in the level, of any variable associated with PCSK9 gene expression, e.g., PCSK9 mRNA level, PCSK9 protein level, or lipid levels. This level may be assessed in an individual cell or in a group of cells, including, for example, a sample derived from a subject.

Inhibition may be assessed by a decrease in an absolute or relative level of one or more variables that are associated with PCSK9 expression compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the disclosed embodiments, expression of a PCSK9 gene is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%. at least about 95%, at least about 96%, at least about 97%, at least about 98%, or at least about 99%.

Inhibition of the expression of a PCSK9 gene may be manifested by a reduction of the amount of mRNA expressed by a first cell or group of cells (such cells may be present, for example, in a sample derived from a subject) in which a PCSK9 gene is transcribed and which has or have been treated (e.g., by contacting the cell or cells with an RNAi agent of the disclosed embodiments, or by administering an RNAi agent of the disclosed embodiments to a subject in which the cells are or were present) such that the expression of a PCSK9 gene is inhibited, as compared to a second cell or group of cells substantially identical to the first cell or group of cells but which has not or have not been so treated (control cell(s)).

Alternatively, inhibition of the expression of a PCSK9 gene may be assessed in terms of a reduction of a parameter that is functionally linked to PCSK9 gene expression, e.g., PCSK9 protein expression, such as lipid levels, cholesterol levels, e.g., LDLc levels. PCSK9 gene silencing may be determined in any cell expressing PCSK9, either constitutively or by genomic engineering, and by any assay known in the art. The liver is the major site of PCSK9 expression. Other significant sites of expression include the pancreas, kidney, and intestines.

Inhibition of the expression of a PCSK9 protein may be manifested by a reduction in the level of the PCSK9 protein that is expressed by a cell or group of cells (e.g., the level of protein expressed in a sample derived from a subject). As explained above for the assessment of mRNA suppression, the inhibition of protein expression levels in a treated cell or group of cells may similarly be expressed as a percentage of the level of protein in a control cell or group of cells.

A control cell or group of cells that may be used to assess the inhibition of the expression of a PCSK9 gene includes a cell or group of cells that has not yet been contacted with an RNAi agent of the disclosed embodiments. For example, the control cell or group of cells may be derived from an individual subject (e.g., a human or animal subject) prior to treatment of the subject with an RNAi agent.

The level of PCSK9 mRNA that is expressed by a cell or group of cells may be determined using any method known in the art for assessing mRNA expression. In one embodiment, the level of expression of PCSK9 in a sample is determined by detecting a transcribed polynucleotide, or portion thereof, e.g., mRNA of the PCSK9 gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, RNase protection assays (Melton et al., Nuc. Acids Res. 12:7035), Northern blotting, in situ hybridization, and microarray analysis.

In one embodiment, the level of expression of PCSK9 is determined using a nucleic acid probe. The term “probe”, as used herein, refers to any molecule that is capable of selectively binding to a specific PCSK9. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction (PCR) analyses and probe arrays. One method for the determination of mRNA levels involves contacting the isolated mRNA with a nucleic acid molecule (probe) that can hybridize to PCSK9 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in determining the level of PCSK9 mRNA.

An alternative method for determining the level of expression of PCSK9 in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosed embodiments, the level of expression of PCSK9 is determined by quantitative fluorogenic RT-PCR (i.e., the TaqMan™ System).

The expression levels of PCSK9 mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of PCSK9 expression level may also comprise using nucleic acid probes in solution.

In preferred embodiments, the level of mRNA expression is assessed using branched DNA (bDNA) assays or real time PCR (qPCR). The use of these methods is described and exemplified in the Examples presented herein.

The level of PCSK9 protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, Western blotting, radioimmunoassay (RIA), enzyme-linked immunosorbent assays (ELISAs), immunofluorescent assays, electrochemiluminescence assays, and the like.

In some embodiments of the methods of the disclosed embodiments, the RNAi agent is administered to a subject such that the RNAi agent is delivered to a specific site within the subject. The inhibition of expression of PCSK9 may be assessed using measurements of the level or change in the level of PCSK9 mRNA or PCSK9 protein in a sample derived from fluid or tissue from the specific site within the subject. In preferred embodiments, the site is the liver. The site may also be a subsection or subgroup of cells from any one of the aforementioned sites. The site may also include cells that express a particular type of receptor.

Treatment or Prevention pcsk9-Related Diseases

The disclosed embodiments also provides methods for treating or preventing diseases and conditions that can be modulated by down regulating PCSK9 gene expression. For example, the compositions described herein can be used to treat lipidemia, e.g., a hyperlipidemia and other forms of lipid imbalance such as hypercholesterolemia, hypertriglyceridemia and the pathological conditions associated with these disorders such as heart and circulatory diseases. Other diseases and conditions that can be modulated by down regulating PCSK9 gene expression include lysosomal storage diseases including, but not limited to, Niemann-Pick disease, Tay-Sachs disease, Lysosomal acid lipase deficiency, and Gaucher Disease. The methods include administering to the subject a therapeutically effective amount or prophylactically effective amount of an RNAi agent of the disclosed embodiments. In some embodiments, the method includes administering an effective amount of a PCSK9 siRNA to a patient having a heterozygous LDLR genotype.

The effect of the decreased PCSK9 gene preferably results in a decrease in LDLc (low density lipoprotein cholesterol) levels in the blood, and more particularly in the serum, of the mammal. In some embodiments, LDLc levels are decreased by at least 10%, 15%, 20%, 25%, 30%, 40%, 50%, 60%, 70%, 80%, 90% or more, as compared to pretreatment levels.

As used herein, a “subject” includes a human or non-human animal, preferably a vertebrate, and more preferably a mammal. A subject may include a transgenic organism. Most preferably, the subject is a human, such as a human suffering from or predisposed to developing a PCSK9-associated disease.

In some embodiments of the methods of the disclosed embodiments, PCSK9 expression is decreased for an extended duration, e.g., at least one week, two weeks, three weeks, or four weeks or longer. For example, in certain instances, expression of the PCSK9 gene is suppressed by at least about 5%, 10%, 15%, 20%, 25%, 30%, 35%, 40%, 45%, or 50% by administration of an iRNA agent described herein. In some embodiments, the PCSK9 gene is suppressed by at least about 60%, 70%, or 80% by administration of the iRNA agent. In some embodiments, the PCSK9 gene is suppressed by at least about 85%, 90%, or 95% by administration of the double-stranded oligonucleotide.

The RNAi agents of the disclosed embodiments may be administered to a subject using any mode of administration known in the art, including, but not limited to subcutaneous, intravenous, intramuscular, intraocular, intrabronchial, intrapleural, intraperitoneal, intraarterial, lymphatic, cerebrospinal, and any combinations thereof. In preferred embodiments, the agents are administered subcutaneously.

In some embodiments, the administration is via a depot injection. A depot injection may release the RNAi agent in a consistent way over a prolonged time period. Thus, a depot injection may reduce the frequency of dosing needed to obtain a desired effect, e.g., a desired inhibition of PCSK9, or a therapeutic or prophylactic effect. A depot injection may also provide more consistent serum concentrations. Depot injections may include subcutaneous injections or intramuscular injections. In preferred embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In preferred embodiments, the infusion pump is a subcutaneous infusion pump. In other embodiments, the pump is a surgically implanted pump that delivers the RNAi agent to the liver.

Other modes of administration include epidural, intracerebral, intracerebroventricular, nasal administration, intraarterial, intracardiac, intraosseous infusion, intrathecal, and intravitreal, and pulmonary. The mode of administration may be chosen based upon whether local or systemic treatment is desired and based upon the area to be treated. The route and site of administration may be chosen to enhance targeting.

The method includes administering an iRNA agent, e.g., a dose sufficient to depress levels of PCSK9 mRNA for at least 5, more preferably 7, 10, 14, 21, 25, 30 or 40 days; and optionally, administering a second single dose of dsRNA, wherein the second single dose is administered at least 5, more preferably 7, 10, 14, 21, 25, 30 or 40 days after the first single dose is administered, thereby inhibiting the expression of the PCSK9 gene in a subject.

In one embodiment, doses of iRNA agent of the disclosed embodiments are administered not more than once every four weeks, not more than once every three weeks, not more than once every two weeks, or not more than once every week. In another embodiment, the administrations can be maintained for one, two, three, or six months, or one year or longer.

In another embodiment, administration can be provided when Low Density Lipoprotein cholesterol (LDLc) levels reach or surpass a predetermined minimal level, such as greater than 70 mg/dL, 130 mg/dL, 150 mg/dL, 200 mg/dL, 300 mg/dL, or 400 mg/dL.

In general, the iRNA agent does not activate the immune system, e.g., it does not increase cytokine levels, such as TNF-alpha or IFN-alpha levels. For example, when measured by an assay, such as an in vitro PBMC assay, such as described herein, the increase in levels of TNF-alpha or IFN-alpha, is less than 30%, 20%, or 10% of control cells treated with a control dsRNA, such as a dsRNA that does not target PCSK9.

For example, a subject can be administered a therapeutic amount of an iRNA agent, such as 0.5 mg/kg, 1.0 mg/kg, 1.5 mg/kg, 2.0 mg/kg, or 2.5 mg/kg dsRNA. The iRNA agent can be administered by intravenous infusion over a period of time, such as over a 5 minute, 10 minute, 15 minute, 20 minute, or 25 minute period. The administration is repeated, for example, on a regular basis, such as biweekly (i.e., every two weeks) for one month, two months, three months, four months or longer. After an initial treatment regimen, the treatments can be administered on a less frequent basis. For example, after administration biweekly for three months, administration can be repeated once per month, for six months or a year or longer. Administration of the iRNA agent can reduce PCSK9 levels, e.g., in a cell, tissue, blood, urine or other compartment of the patient by at least 10%, at least 15%, at least 20%, at least 25%, at least 30%, at least 40%, at least 50%, at least 60%, at least 70%, at least 80% or at least 90% or more.

Before administration of a full dose of the iRNA agent, patients can be administered a smaller dose, such as a 5%>infusion reaction, and monitored for adverse effects, such as an allergic reaction, or for elevated lipid levels or blood pressure. In another example, the patient can be monitored for unwanted immunostimulatory effects, such as increased cytokine (e.g., TNF-alpha or INF-alpha) levels.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given iRNA agent of the disclosed embodiments or formulation of that iRNA agent can also be judged using an experimental animal model for the given disease as known in the art. When using an experimental animal model, efficacy of treatment is evidenced when a statistically significant reduction in a marker or symptom is observed.

In one embodiment, the RNAi agent is administered at a dose of between about 0.25 mg/kg to about 50 mg/kg, e.g., between about 0.25 mg/kg to about 0.5 mg/kg, between about 0.25 mg/kg to about 1 mg/kg, between about 0.25 mg/kg to about 5 mg/kg, between about 0.25 mg/kg to about 10 mg/kg, between about 1 mg/kg to about 10 mg/kg, between about 5 mg/kg to about 15 mg/kg, between about 10 mg/kg to about 20 mg/kg, between about 15 mg/kg to about 25 mg/kg, between about 20 mg/kg to about 30 mg/kg, between about 25 mg/kg to about 35 mg/kg, or between about 40 mg/kg to about 50 mg/kg.

In some embodiments, the RNAi agent is administered at a dose of about 0.25 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg or about 50 mg/kg. In one embodiment, iRNA agent is administered at a dose of about 25 mg/kg.

The dose of an RNAi agent that is administered to a subject may be tailored to balance the risks and benefits of a particular dose, for example, to achieve a desired level of PCSK9 gene suppression (as assessed, e.g., based on PCSK9 mRNA suppression, PCSK9 protein expression, or a reduction in lipid levels) or a desired therapeutic or prophylactic effect, while at the same time avoiding undesirable side effects.

In some embodiments, the RNAi agent is administered in a dosing regimen that includes a “loading phase” of closely spaced administrations that may be followed by a “maintenance phase”, in which the RNAi agent is administered at longer spaced intervals. In one embodiment, the loading phase comprises five daily administrations of the RNAi agent during the first week. In another embodiment, the maintenance phase comprises one or two weekly administrations of the RNAi agent. In a further embodiment, the maintenance phase lasts for 5 weeks. In one embodiment, the loading phase comprises administration of a dose of 2 mg/kg, 1 mg/kg or 0.5 mg/kg five times a week. In another embodiment, the maintenance phase comprises administration of a dose of 2 mg/kg, 1 mg/kg or 0.5 mg/kg once, twice, or three times weekly, once every two weeks, once every three weeks, once a month, once every two months, once every three months, once every four months, once every five months, or once every six months.

Any of these schedules may optionally be repeated for one or more iterations. The number of iterations may depend on the achievement of a desired effect, e.g., the suppression of a PCSK9 gene, and/or the achievement of a therapeutic or prophylactic effect, e.g., reducing serum cholesterol levels or reducing a symptom of hypercholesterolemia.

In further embodiments, administration of a siRNA is administered in combination an additional therapeutic agent. The siRNA and an additional therapeutic agent can be administered in combination in the same composition, e.g., parenterally, or the additional therapeutic agent can be administered as part of a separate composition or by another method described herein.

Examples of additional therapeutic agents include those known to treat an agent known to treat a lipid disorders, such as hypercholesterolemia, atherosclerosis or dyslipidemia. For example, a siRNA featured in the disclosed embodiments can be administered with, e.g., an HMG-CoA reductase inhibitor (e.g., a statin), a fibrate, a bile acid sequestrant, niacin, an antiplatelet agent, an angiotensin converting enzyme inhibitor, an angiotensin II receptor antagonist (e.g., losartan potassium, such as Merck & Co.'s Cozaar®), an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant (e.g., AGI-1067, from Atherogenics), a glycoprotein IIb/IIIa inhibitor, aspirin or an aspirin-like compound, an IBAT inhibitor (e.g., S-8921, from Shionogi), a squalene synthase inhibitor, or a monocyte chemoattractant protein (MCP)-I inhibitor. Exemplary HMG-CoA reductase inhibitors include atorvastatin (Pfizer's Lipitor®/Tahor/Sortis/Torvast/Cardyl), pravastatin (Bristol-Myers Squibb's Pravachol, Sankyo's Mevalotin/Sanaprav), simvastatin (Merck's Zocor®/Sinvacor, Boehringer Ingelheim's Denan, Banyu's Lipovas), lovastatin (Merck's Mevacor/Mevinacor, Bexal's Lovastatina, Cepa; Schwarz Pharma's Liposcler), fluvastatin (Novartis' Lescol®/Locol/Lochol, Fujisawa's Cranoc, Solvay's Digaril), cerivastatin (Bayer's Lipobay/GlaxoSmithKline's Baycol), rosuvastatin (AstraZeneca's Crestor®), and pitivastatin (itavastatin/risivastatin) (Nissan Chemical, Kowa Kogyo, Sankyo, and Novartis). Exemplary fibrates include, e.g., bezafibrate (e.g., Roche's Befizal®/Cedur®/Bezalip®, Kissei's Bezatol), clofibrate (e.g., Wyeth's Atromid-S®), fenofibrate (e.g., Fournier's Lipidil/Lipantil, Abbott's Tricor®, Takeda's Lipantil, generics), gemfibrozil (e.g., Pfizer's Lopid/Lipur) and ciprofibrate (Sanofi-Synthelabo's Modalim®). Exemplary bile acid sequestrants include, e.g., cholestyramine (Bristol-Myers Squibb's Questran® and Questran Light™), colestipol (e.g., Pharmacia's Colestid), and colesevelam (Genzyme/Sankyo's WelChol™). Exemplary niacin therapies include, e.g., immediate release formulations, such as Aventis' Nicobid, Upsher-Smith's Niacor, Aventis' Nicolar, and Sanwakagaku's Perycit. Niacin extended release formulations include, e.g., Kos Pharmaceuticals' Niaspan and Upsher-Smith's SIo-Niacin. Exemplary antiplatelet agents include, e.g., aspirin (e.g., Bayer's aspirin), clopidogrel (Sanofi-Synthelabo/Bristol-Myers Squibb's Plavix), and ticlopidine (e.g., Sanofi-Synthelabo's Ticlid and Daiichi's Panaldine). Other aspirin-like compounds useful in combination with a dsRNA targeting PCSK9 include, e.g., Asacard (slow-release aspirin, by Pharmacia) and Pamicogrel (Kanebo/Angelini Ricerche/CEPA). Exemplary angiotensin-converting enzyme inhibitors include, e.g., ramipril (e.g., Aventis' Altace) and enalapril (e.g., Merck & Co.'s Vasotec). Exemplary acyl CoA cholesterol acetyltransferase (AC AT) inhibitors include, e.g., avasimibe (Pfizer), eflucimibe (BioMsrieux Pierre Fabre/Eli Lilly), CS-505 (Sankyo and Kyoto), and SMP-797 (Sumito). Exemplary cholesterol absorption inhibitors include, e.g., ezetimibe (Merck/Schering-Plough Pharmaceuticals Zetia®) and Pamaqueside (Pfizer). Exemplary CETP inhibitors include, e.g., Torcetrapib (also called CP-529414, Pfizer), JTT-705 (Japan Tobacco), and CETi-I (Avant Immunotherapeutics). Exemplary microsomal triglyceride transfer protein (MTTP) inhibitors include, e.g., implitapide (Bayer), R-103757 (Janssen), and CP-346086 (Pfizer). Other exemplary cholesterol modulators include, e.g., NO-1886 (Otsuka/TAP Pharmaceutical), CI-1027 (Pfizer), and WAY-135433 (Wyeth-Ayerst).

Exemplary bile acid modulators include, e.g., HBS-107 (Hisamitsu/Banyu), Btg-511 (British Technology Group), BARI-1453 (Aventis), S-8921 (Shionogi), SD-5613 (Pfizer), and AZD-7806 (AstraZeneca). Exemplary peroxisome proliferation activated receptor (PPAR) agonists include, e.g., tesaglitazar (AZ-242) (AstraZeneca), Netoglitazone (MCC-555) (Mitsubishi/Johnson & Johnson), GW-409544 (Ligand Pharmaceuticals/GlaxoSmithKline), GW-501516 (Ligand Pharmaceuticals/GlaxoSmithKline), LY-929 (Ligand Pharmaceuticals and Eli Lilly), LY-465608 (Ligand Pharmaceuticals and Eli Lilly), LY-518674 (Ligand Pharmaceuticals and Eli Lilly), and MK-767 (Merck and Kyorin). Exemplary gene-based therapies include, e.g., AdGWEGF 121.10 (GenVec), ApoA1 (UCB Pharma/Groupe Fournier), EG-004 (Trinam) (Ark Therapeutics), and ATP-binding cassette transporter-A1 (ABCA1) (CV Therapeutics/Incyte, Aventis, Xenon). Exemplary Glycoprotein IIb/IIIa inhibitors include, e.g., roxifiban (also called DMP754, Bristol-Myers Squibb), Gantofiban (Merck KGaA/Yamanouchi), and Cromafiban (Millennium Pharmaceuticals). Exemplary squalene synthase inhibitors include, e.g., BMS-1884941 (Bristol-Myers Squibb), CP-210172 (Pfizer), CP-295697 (Pfizer), CP-294838 (Pfizer), and TAK-475 (Takeda). An exemplary MCP-I inhibitor is, e.g., RS-504393 (Roche Bioscience). The anti-atherosclerotic agent BO-653 (Chugai Pharmaceuticals), and the nicotinic acid derivative Nyclin (Yamanouchi Pharmaceuticals) are also appropriate for administering in combination with a dsRNA featured in the disclosed embodiments. Exemplary combination therapies suitable for administration with a dsRNA targeting PCSK9 include, e.g., advicor (Niacin/lovastatin from Kos Pharmaceuticals), amlodipine/atorvastatin (Pfizer), and ezetimibe/simvastatin (e.g., Vytorin® 10/10, 10/20, 10/40, and 10/80 tablets by Merck/Schering-Plough Pharmaceuticals). Agents for treating hypercholesterolemia, and suitable for administration in combination with a dsRNA targeting PCSK9 include, e.g., lovastatin, niacin Altoprev® Extended-Release Tablets (Andrx Labs), lovastatin Caduet® Tablets (Pfizer), amlodipine besylate, atorvastatin calcium Crestor® Tablets (AstraZeneca), rosuvastatin calcium Lescol® Capsules (Novartis), fluvastatin sodium Lescol® (Reliant, Novartis), fluvastatin sodium Lipitor® Tablets (Parke-Davis), atorvastatin calcium Lofibra® Capsules (Gate), Niaspan Extended-Release Tablets (Kos), niacin Pravachol Tablets (Bristol-Myers Squibb), pravastatin sodium TriCor® Tablets (Abbott), fenofibrate Vytorin® 10/10 Tablets (Merck/Schering-Plough Pharmaceuticals), ezetimibe, simvastatin WelChol Tablets (Sankyo), colesevelam hydrochloride Zetia® Tablets (Schering), ezetimibe Zetia® Tablets (Merck/Schering-Plough Pharmaceuticals), and ezetimibe Zocor® Tablets (Merck).

In one embodiment, an iRNA agent is administered in combination with an ezetimibe/simvastatin combination (e.g., Vytorin® (Merck/Schering-Plough Pharmaceuticals)). In one embodiment, the iRNA agent is administered to the patient, and then the additional therapeutic agent is administered to the patient (or vice versa). In another embodiment, the iRNA agent and the additional therapeutic agent are administered at the same time.

In another aspect, the disclosed embodiments include a method of instructing an end user, e.g., a caregiver or a subject, on how to administer an iRNA agent described herein. The method includes, optionally, providing the end user with one or more doses of the iRNA agent, and instructing the end user to administer the iRNA agent on a regimen described herein, thereby instructing the end user.

In one aspect, the disclosed embodiments provide a method of treating a patient by selecting a patient on the basis that the patient is in need of LDL lowering, LDL lowering without lowering of HDL, ApoB lowering, or total cholesterol lowering. The method includes administering to the patient a siRNA in an amount sufficient to lower the patient's LDL levels or ApoB levels, e.g., without substantially lowering HDL levels.

Genetic predisposition plays a role in the development of target gene associated diseases, e.g., hyperlipidemia. Therefore, a patient in need of a siRNA can be identified by taking a family history, or, for example, screening for one or more genetic markers or variants. Examples of genes involved in hyperlipidemia include but are not limited to, e.g., LDL receptor (LDLR), the apoliproteins (ApoA1, ApoB, ApoE, and the like), Cholesteryl ester transfer protein (CETP), Lipoprotein lipase (LPL), hepatic lipase (LIPC), Endothelial lipase (EL), Lecithinxholesteryl acyltransferase (LCAT).

A healthcare provider, such as a doctor, nurse, or family member, can take a family history before prescribing or administering an iRNA agent of the disclosed embodiments. In addition, a test may be performed to determine a genotype or phenotype. For example, a DNA test may be performed on a sample from the patient, e.g., a blood sample, to identify the PCSK9 genotype and/or phenotype before a PCSK9 dsRNA is administered to the patient. In another embodiment, a test is performed to identify a related genotype and/or phenotype, e.g., a LDLR genotype. Example of genetic variants with the LDLR gene can be found in the art, e.g., in the following publications which are incorporated by reference: Costanza et al (2005) Am J Epidemiol. 15; 161(8):714-24; Yamada et al. (2008) J Med Genet. January; 45(1):22-8, Epub 2007 Aug. 31; and Boes et al (2009) Exp. Gerontol 44: 136-160, Epub 2008 Nov. 17.

### EXAMPLES

### Example 1. Synthesis and Characterization of Azido-PDoV1 (1)

Peptide Azido-PDoV1 (sequence 1-1E1H{LYS(PEG4-N3)}FIHCKHHH (SEQ ID NO: 4)) was synthesized using an automated peptide synthesizer using a commercial service and using standard amino acids and lysine-PEG4-N3 modifier in the sequence. The peptide was purified by C-18 reverse phase HPLC and characterized by mass spectrometry. ¹H NMR and mass spectrometry were consistent with the expected structure.

### Example 2. Synthesis of PDoV2 and Azido-PDoV2

Synthesis of PDoV2 (2), sequence HHHKHHCRHHH (SEQ ID NO: 5). Peptide PDoV2 (HHHKHHCRHHH (SEQ ID NO: 5)) was synthesized by the automated peptide synthesizer by contracted service and using standard amino acids in the sequence. The peptide was purified by C-18 reverse phase HPLC and characterized by mass spectrum (shown below). ¹H NMR and mass spectrometry were consistent with the expected structure.

Synthesis of Azido-PDoV2 (3) (Discloses SEQ ID NOS 5 and 4, respectively, in order of appearance):

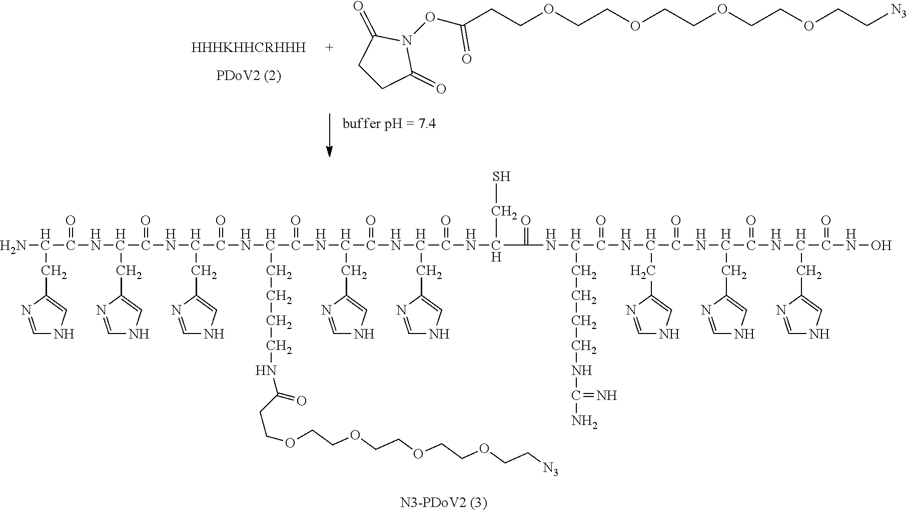 [SMILES: [N-]=[N+]=NCCOCCOCCOCCOCCC(=O)ON1C(=O)CCC1=O ; [N-]=[N+]=NCCOCCOCCOCCOCCC(=O)NCCCCC(NC(=O)C(Cc1c[nH]cn1)NC(=O)C(Cc1c[nH]cn1)NC(=O)C(N)Cc1c[nH]cn1)C(=O)NC(Cc1c[nH]cn1)C(=O)NC(Cc1c[nH]cn1)C(=O)NC(CS)C(=O)NC(CCCCNC(=N)N)C(=O)NC(Cc1c[nH]cn1)C(=O)NC(Cc1c[nH]cn1)C(=O)NC(Cc1c[nH]cn1)C(=O)NO]

The azide linker was attached to the Peptide Docking Vehicle 2 (PDoV2) via amide bond formation between the ester activated carboxylic acid of the azide linker and the primary amine of the Lysine side chain of PDoV2 (2) to form compound 3. PDoV2 peptide HHHKHHCRHHH (SEC) ID NO: 5) (42 mg, 0.0280 mmol) was suspended in 1.0 mL DMF. Triethyl amine (39 uL, 10 eq) was added and the mixture was stirred at room temperature for 20 minutes. A solution of Azido-Peg4-NHS ester (54 mg, 0.140 mmol, 5 eq) in 20 uL of DMF was added to the reaction mixture. The reaction mixture slowly turned into a clear solution over 30 minutes and was stirred further at room temperature for 16 hours. The TLC profile of the reaction mixture was monitored by HPLC profile of full complete conversion of PDoV2.

The reaction mixture was quenched with water (200 μL), concentrated using a rotary evaporator and the crude material was purified through HPLC on semi-prep RP-C₁₈ column using an increasing gradient of 10-90% of Buffer B (0.1% TFA in Acetonitrile). Azido-PDoV2 (2) was isolated as the major product with a retention time between 10.5 and 11.5 minutes. Sample fractions were lyophilized resulting in a clear residual oil of compound 2 (44 mg, 88% yield). Its proton and MS analysis were as follows: ¹H NMR (400 MHz, D2O, FIG. 2) δ 8.74 (brd d, 8H) and δ 7.35 (brd d, 8H) are consistent with aromatic hydrogens associated with 8 histidine tetrazoles in the peptide above δ 6.00 ppm. Methine hydrogens at the alpha carbon of all the 11 amino acids at δ 4.75-4.30 (br, t, 11H), ethylene hydrogens associated with polyethylene group at δ 3.90-3.75 (m, 100H); δ 3.6-2.75 (m, 53H) and δ 1.8-1.3 (m, 12H) ethylene hydrogens associated with the side chain protons of Lysine, arginine and cysteine.

### Example 3. Synthesis and Characterization of Azido-PDoV3 Peptide (4)

Peptide Azido-PDoV3 ({LYS(PEG4-N3}HHHCHH) (SEQ ID NO: 6) was synthesized using solid-phase automated synthesis using standard amino acids plus lysine-PEG4-N3 modifier in the sequence. The peptide was purified by C-18 reverse phase HPLC and characterized by H¹NMR and mass spectrum (shown below). ¹H NMR and mass spectrometry were consistent with the expected structure.

### Example 4. Synthesis and Characterization of PDoV1-GalNAc3 (5) (Discloses SEQ ID NOS 7-8, Respectively, in Order of Appearance)

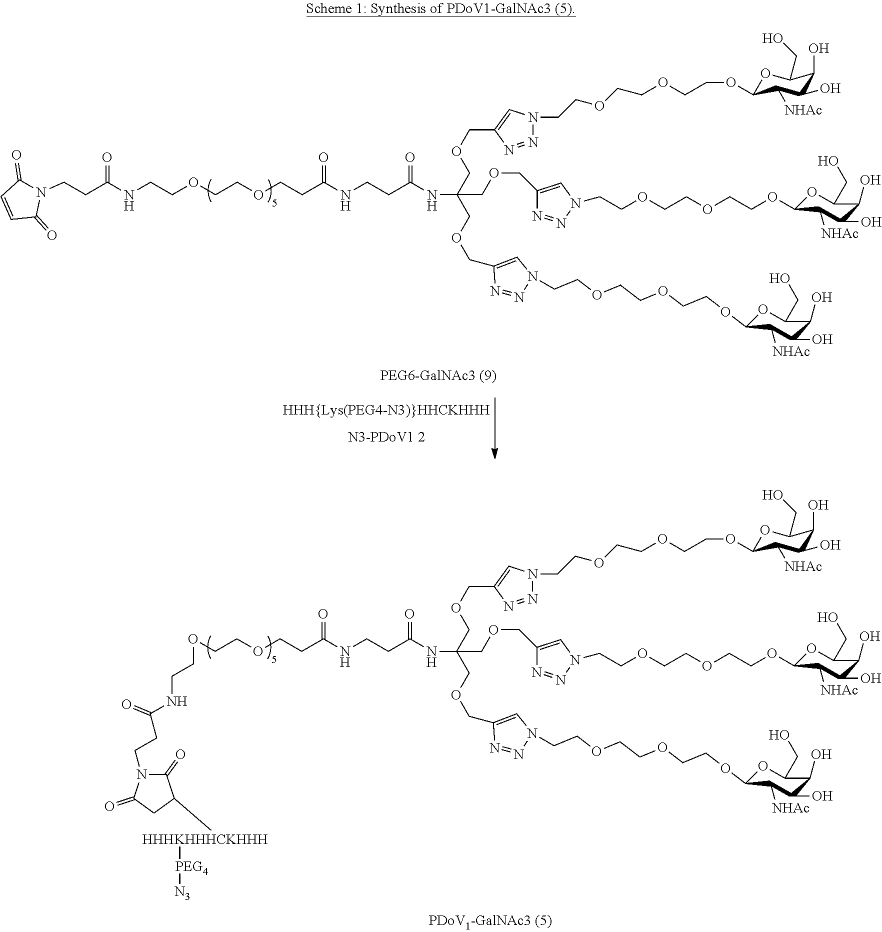

PEG6-GalNAc3 (9) (3.0 mg, 1.56 μmol) in dry DMF (400 uL) was added to the solution of N3-PDoV1 2 (3.54 mg, 2.03 μmol) in phosphate buffer (1 mL, pH=7.4). The resulting mixture was stirred at 25° C. under nitrogen overnight. After the solvent was removed under reduced pressure, the sample was desalted and purified by PD-10 column to provide the pure product PDoV1-GalNAc3 5 (5.1 mg, white solid, yield 90%). The product was analyzed by HPLC using a reverse phase C₁₈ column, gradient elution by solvent 0.1% TFA water and 0.1% acetonitrile. Retention time Rt=4.877min, purity>90%. Mass spectrum analysis (ESI, positive): Calc. for C₁₅₄H₂₄₀N48O55S 3673.7 found 3674.8.

### Example 5. Synthesis and Characterization of PDoV2-GalNAc3 6 and 7 (Discloses SEQ ID NO: 136).

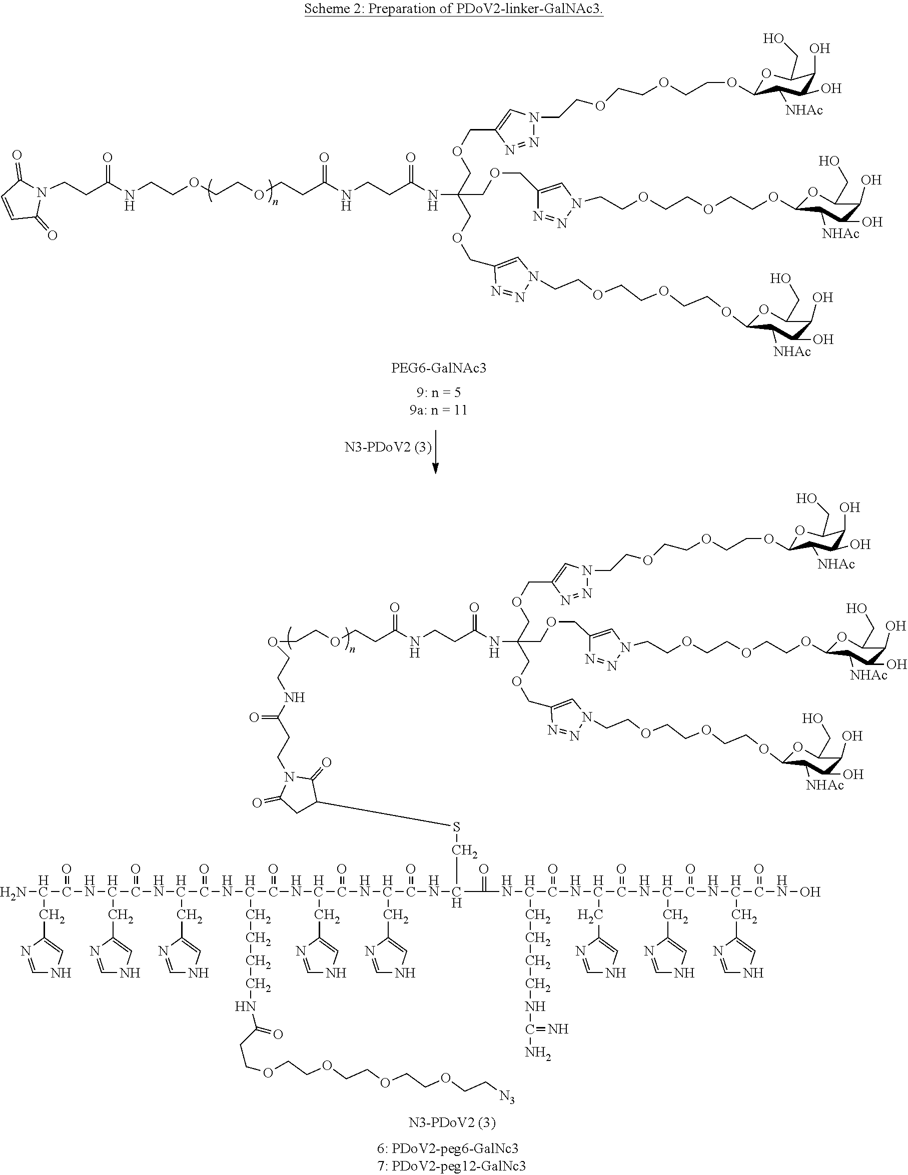

Preparation of PDoV2-Peg6-GalNAc3 (compound 6): The nucleophile, Compound 2 (49.8 mg, 0.0243 mmol) was dissolved in 1.0 mL of degassed PBS buffer at pH 7.4. Trivalent GalNAc-ligand (9) (30.8 mg, 0.0160 mmol) was dissolved and delivered in 400 uL of dry DMF. The reaction mixture was again degassed under dry argon and allowed to stir at room temperature overnight. The reaction mixture was quenched with water (100 μL) and desalted through 1.0 μmol Sephadex Nap column following the Glen Research recommended protocols. The eluent was lyophilized, and the crude material was eluted on HPLC through a semi-prep C₁₈ reverse phase column with increasing gradient of 10-90% of Buffer B (0.1% TFA in Acetonitrile and water. The product had a retention time of 4.0 mins and was isolated as an oil (39 mg, 60% yield). The mass spec of the modified oligos confirmed that our PDoV2-peg6-GalNAc3 construct was successful.

### Example 6. Synthesis of PDoV3-GalNAc3 (8)

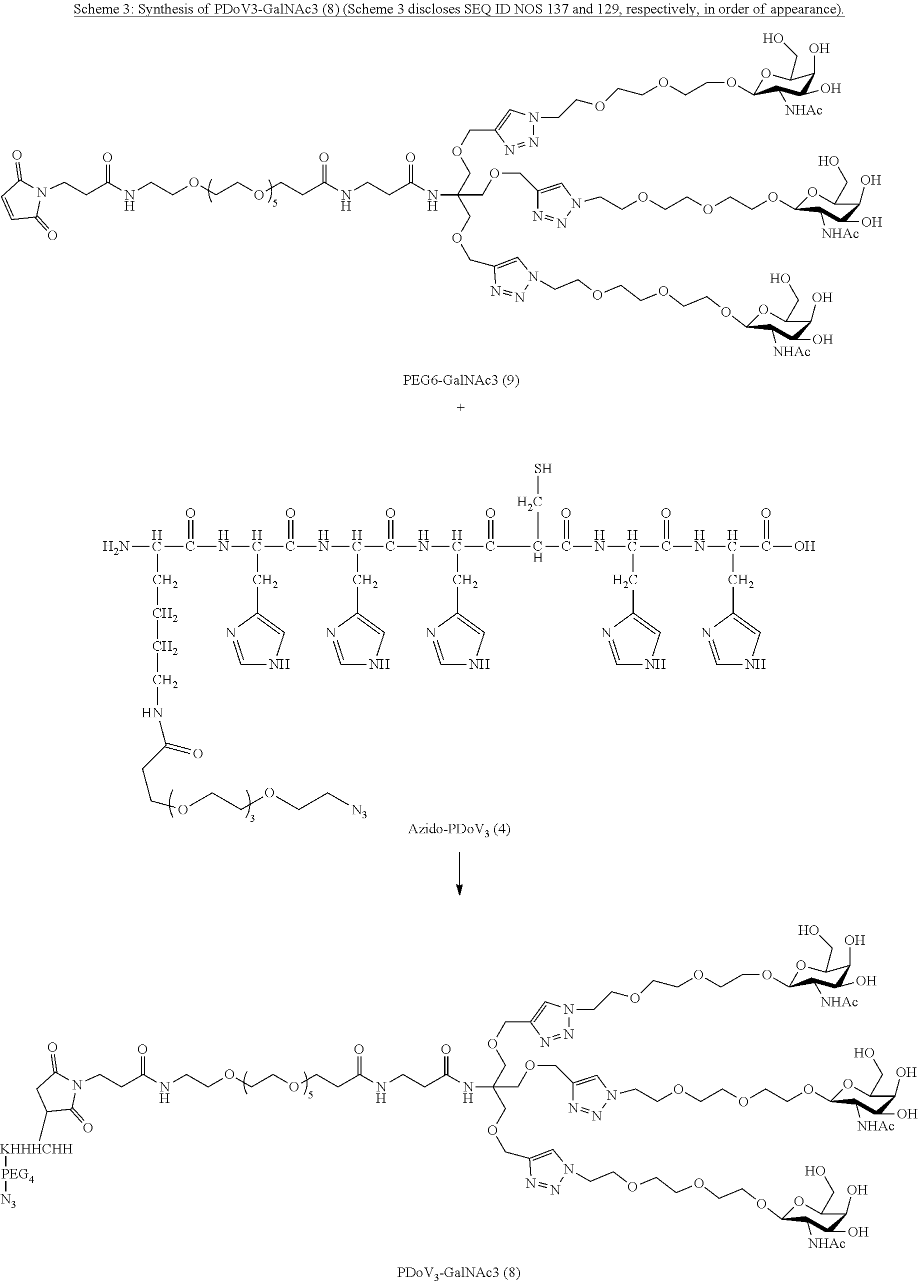

Synthesis of PDoV3-GalNAc3 (compound 8): Azido-PDoV3 compound 4 (47.0 mg, 38.9 μmol) in DMF (1.5 mL) was added to the mixture of trivalent GalNAc (9) (50.0 mg, 25.9 μmol) in phosphate buffer (4 mL, pH 7.4) under nitrogen at 25° C. The resultant reaction mixture was stirred at 25° C. for 12 hours. The reaction was monitored by HPLC until GalNAc 9 was fully consumed. The solvent was removed by lyophilization and the crude material was purified by gel permeation column chromatograph PD-10 column to provide the pure product PDoV3-GalNAc3 compound 8 (70 mg, yield 86.4%). The HPLC was performed on reverse phase C-18 column by gradient elution of solvent 0.1% TFA in water and 0.1% TFA in acetonitrile Rt=5.038 min. MS (ESI, positive mode) Exact Mass: 3134.45 for Formula: C₁₃₀H₂₀₇N₃₇O₅₁S. Found: 3136.35.

### Example 7. Synthesis of Control3-GalNAc3 (11).

Preparation of PDoV3-control3-GalNAc3:

Compound azido-control3 peptide 11 (sequence {LYS(PEG4-N3)}SSSCSS (SEQ ID NO: 9)) (2.6 mg, 2.59 μmol) was dissolved in 1.0 mL of degassed PBS buffer at pH 7.4. GalNAc-ligand (5.0 mg, 2.59 μmol) was dissolved and delivered in 500 uL of dry DMF. The reaction mixture was again degassed under dry argon and allowed to stir at room temperature overnight. The reaction mixture was quenched with water (100 μL) and desalted through 1.0 μmol Sephadex Nap column. Several eluent fractions were collected and lyophilized to afford the desired compound Control3-GalNAc3 10. This compound was analyzed using analytical HPLC C18 RP column with increasing gradient of 10-90% of Buffer B (0.1% TFA in Acetonitrile and water. The product had a retention time of 3.80 min and was isolated as a clear oil (4.9 mg, 67% yield). The mass spec of the modified oligonucleotides confirmed the structure of the PDoV3-Control3-peg6-GalNAc construct.

### Example 8. Synthesis and Characterization of PCSK9-PDoV3-GalNAc3 (12) (Example 8 Discloses SEQ ID NO: 10).

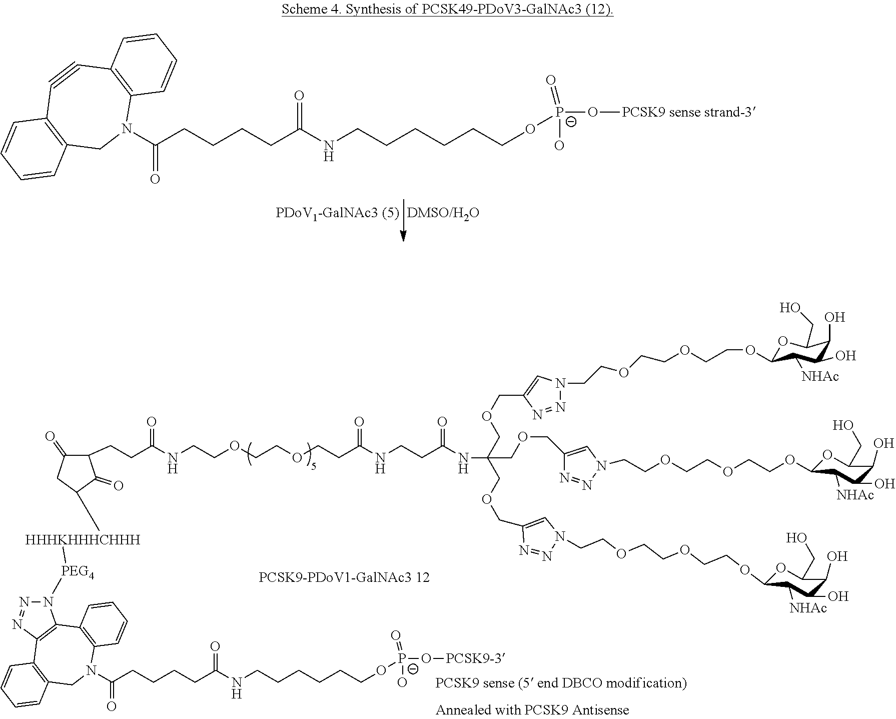

PDoV3-GalNAc3 8 (1.2 μmol) in DMSO (300 μL) was added to a solution of PCSK49-sense-5′-DBCO (1 limo') in RNAse free water (300 4). The resultant mixture was stirred at 25° C. for 2 hours. After solvent was removed under reduced pressure, the crude material was purified by gel permeation column chromatograph PD-10 column to provide the pure product sense PCSK49-PDoV3-GalNAc3 compound 17 (mg, yield 85%). The HPLC was performed on PA200 ion exchange column using phosphate buffer at pH=11, Rt=14.744 min., purity>85%. 1:1 annealing (95° C. for 5min, cool down by around 1° C./min to room temperature, then store under −20° C.) with the antisense strand provided the final conjugate duplex PCSK49-PDoV3-GalNAc3. After performing the 1:1 annealing (95° C. for 5 min, cool down by around 1° C./min to room temperature, then store under −20° C.) with the PCSK49 antisense strand it provided the final conjugate duplex PCSK49-PDoV1-GalNAc3 (12). The product was characterized by HPLC and MS.

### Example 9

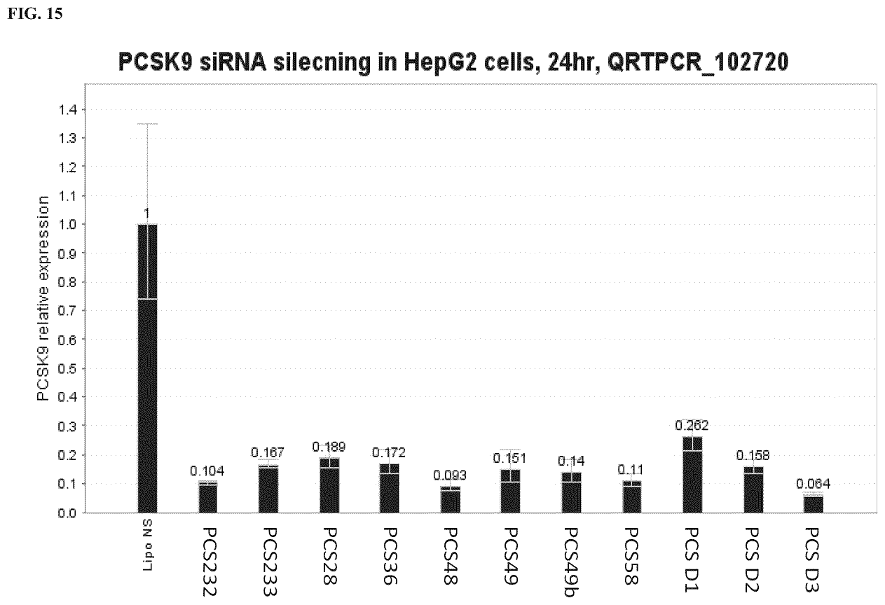

In vitro screening of the PCSK9 siRNA sequence. Knockdown efficacy study of unmodified PCSK9 siRNA. The in vitro experiment was done in HepG2 cells, 1×105 per well in 12 well plate, siRNA final concentration is 50 μM. Transfection duration was 24 hr. There are 11 PCSK9 siRNA samples (PCS232, PCS233, PCS28, PCS36, PCS48, PCS49, PCS49b, PCS58, PCSd1, PCSd2, PCSd3), plus one NS control, no Blank in this setup. QRTPCR: HPRT (as internal control) and PCSK9 primer (F+R, 20 μM each, 0.2 μL per reaction) and probe (10 μM, 0.4 μL per reaction). All nine PCSK9 siRNA showed significant silencing comparing to Lipo NS. See FIG. 15. The mRNA knockdown level is all over 74% to 94%. In case of PCS232, PCS48, PCS58, and PCSd3 the remaining PCSK9 expression level is below 11%. Most of the designed siRNA shown great potency in the mRNA knockdown evaluation experiment.

### Example 10

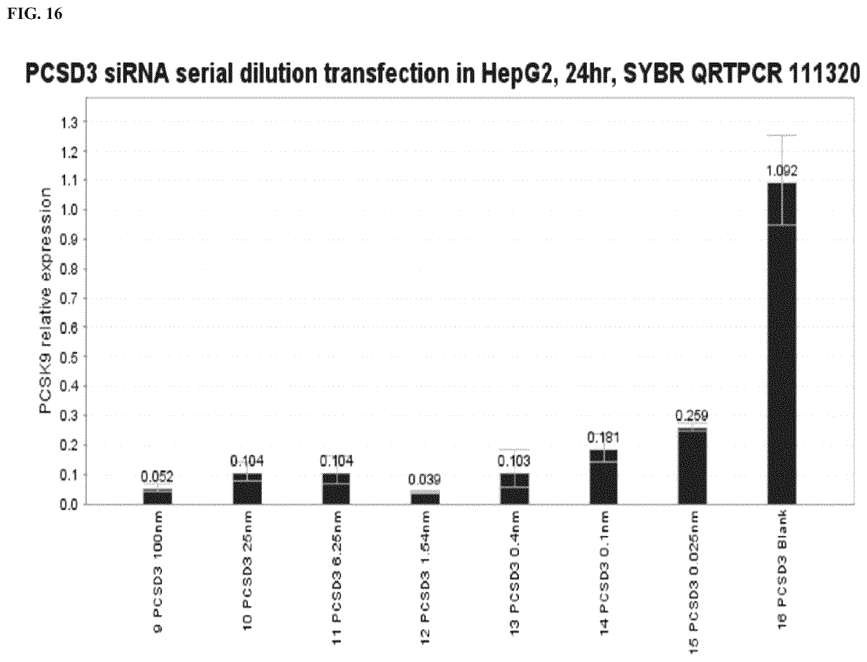

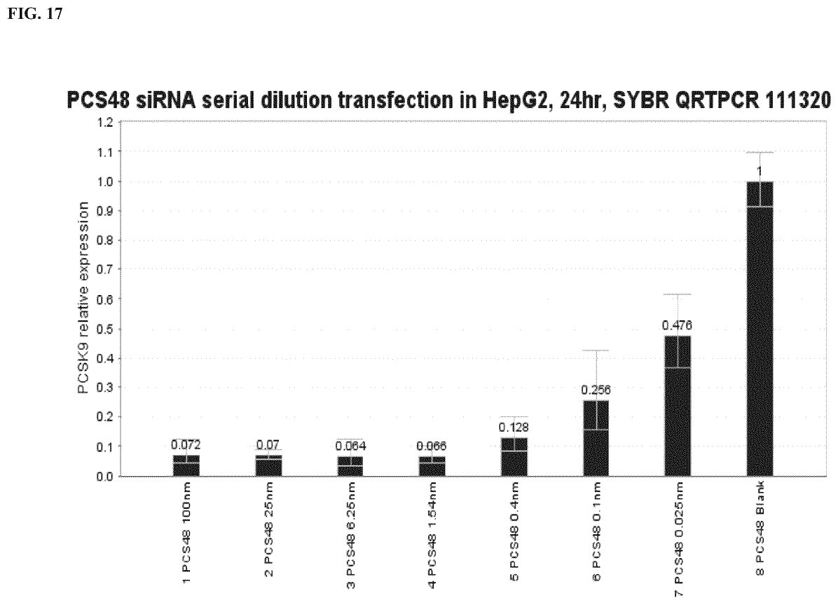

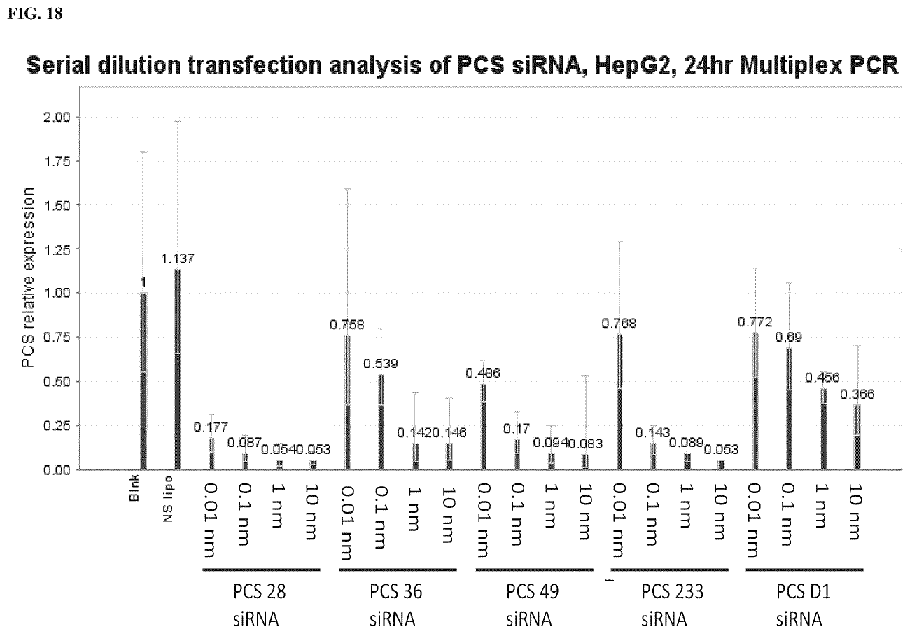

In vitro screening of the PCSK9 siRNA sequence. The in vitro experiment was done in HepG2 cells, 1×105 per well in 12 well plate, siRNA final concentration is 50 μM. Transfection duration was 24 hr. There are 11 samples, plus one NS control, no Blank in this setup. QRTPCR: HPRT (as internal control) and PCSK9 primer (F+R, 20 μM each, 0.2 4 per reaction) and probe (10 μM, 0.4 4 per reaction). PCSKd3 siRNA showed significant silencing comparing to Lipofectamine NS (see FIG. 16, FIG. 17, FIG. 18). The mRNA knockdown level of those PCSK9 siRNA was further evaluated by serial dilution experiment. The IC50 of PCSd3 in inhibition of PCSK9 mRNA is estimated below 25 pM, IC50 of PCS48 is about 25 pM, IC50 of PCS28 is below 10 pM, IC50 of PCS36 is 0.1 nM, IC50 of PCS49 is about 10 pM, IC50 of PCS233 is below 0.1 nM, IC50 of PCSd1 is about 1 nM.

### Example 11

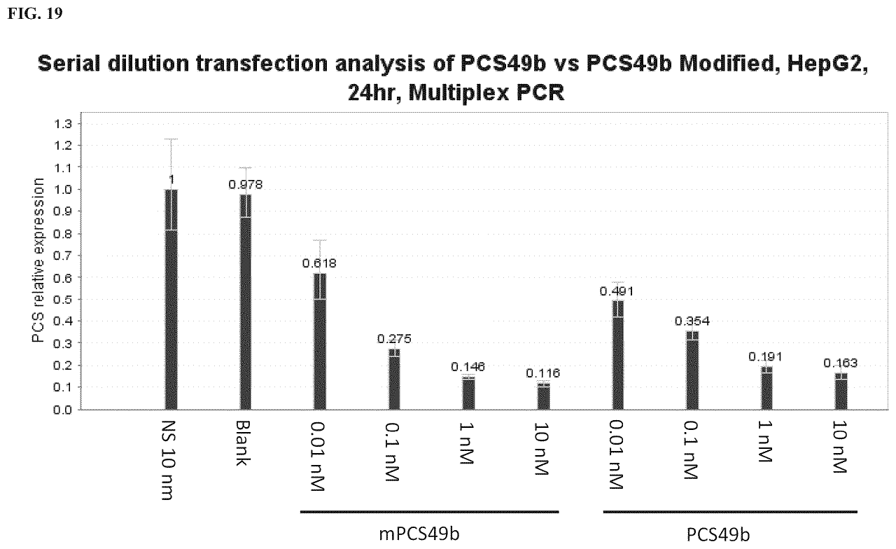

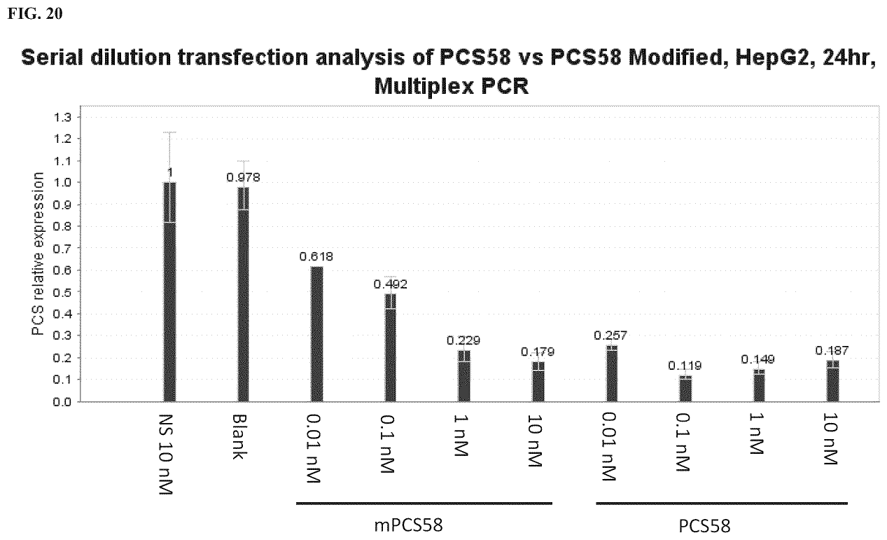

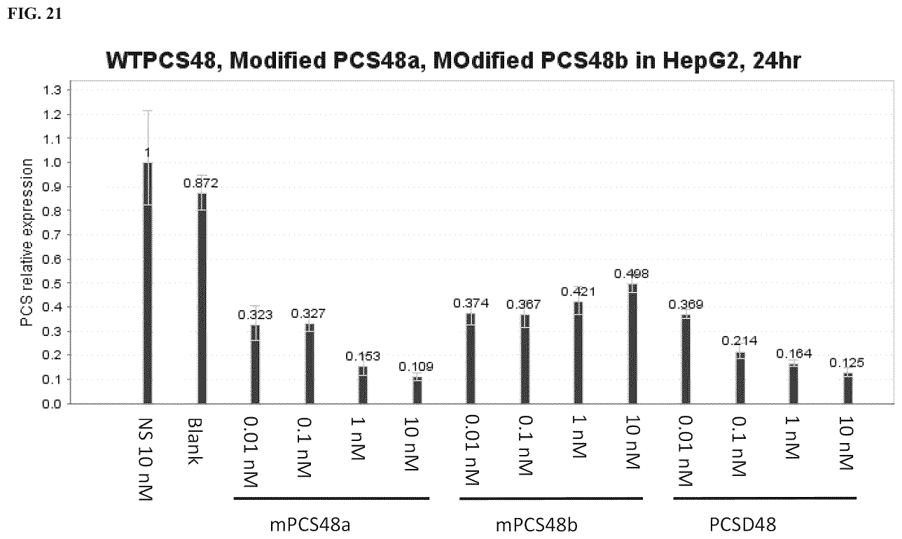

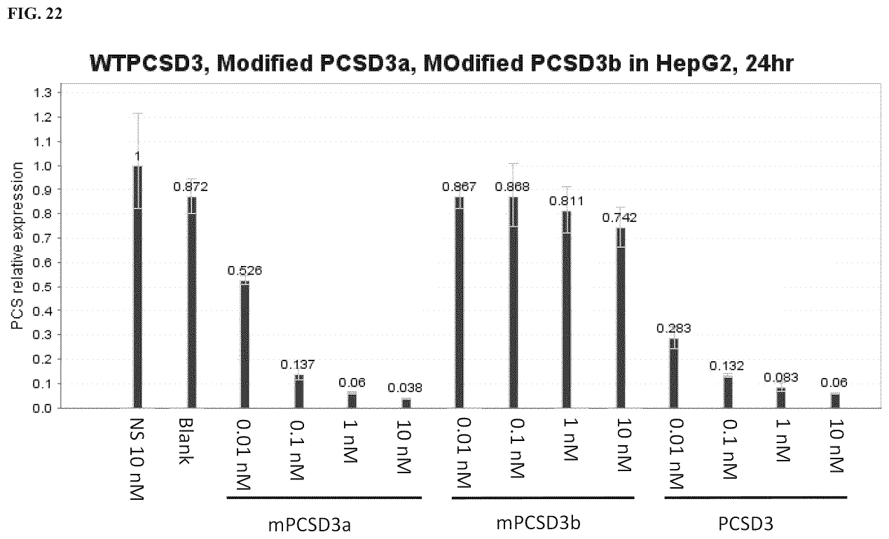

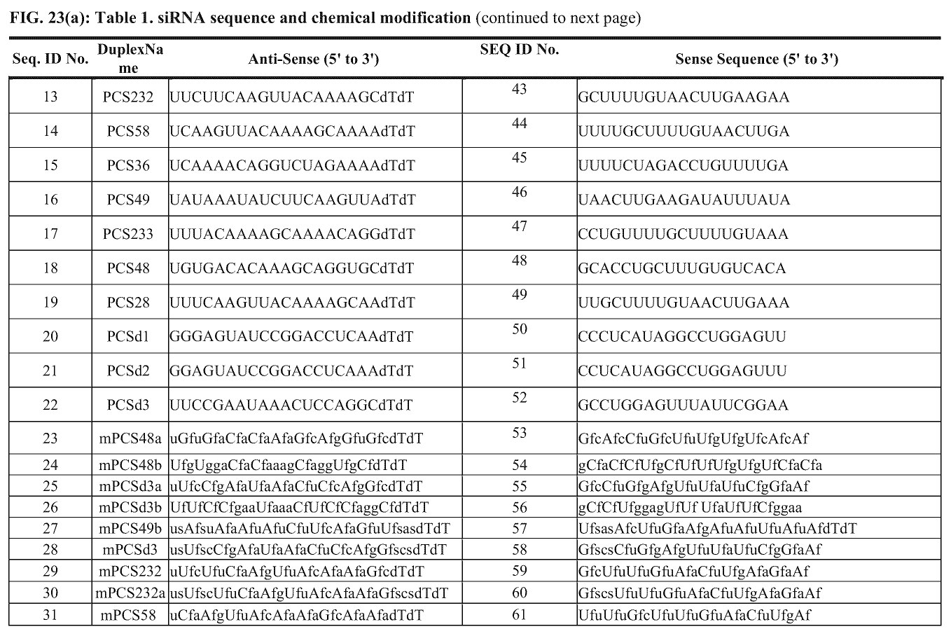

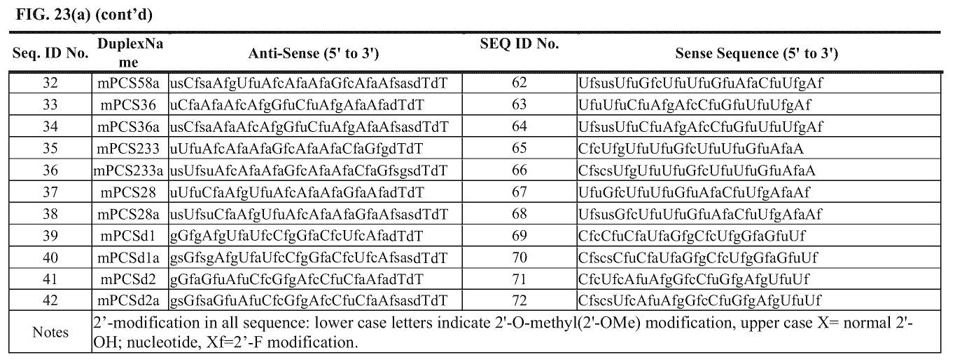

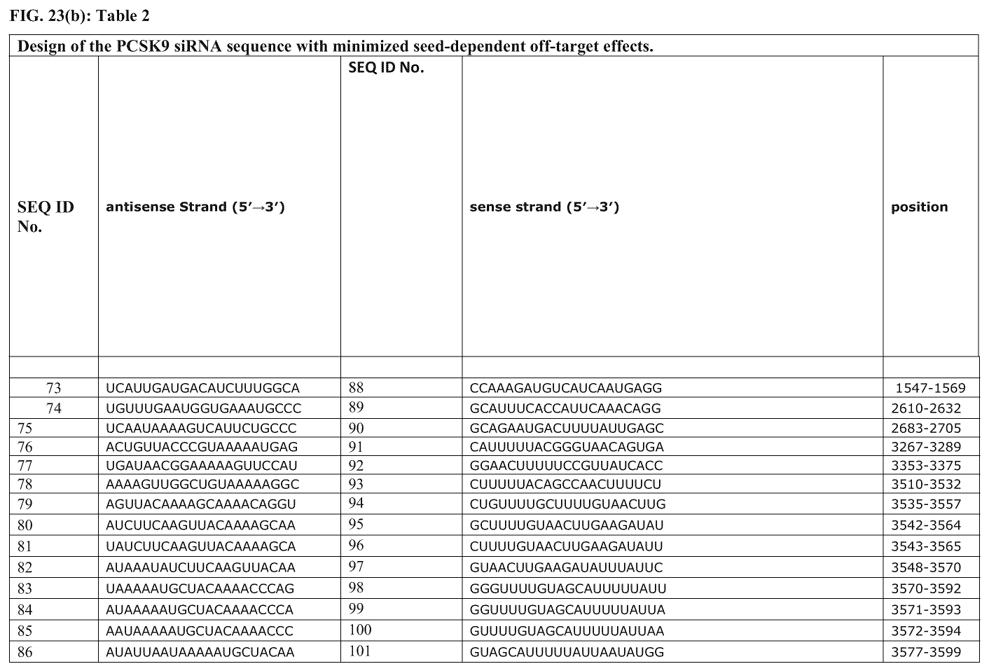

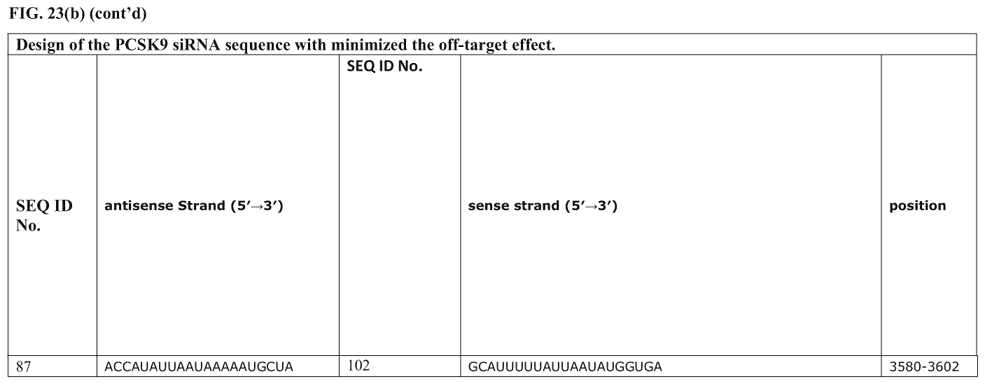

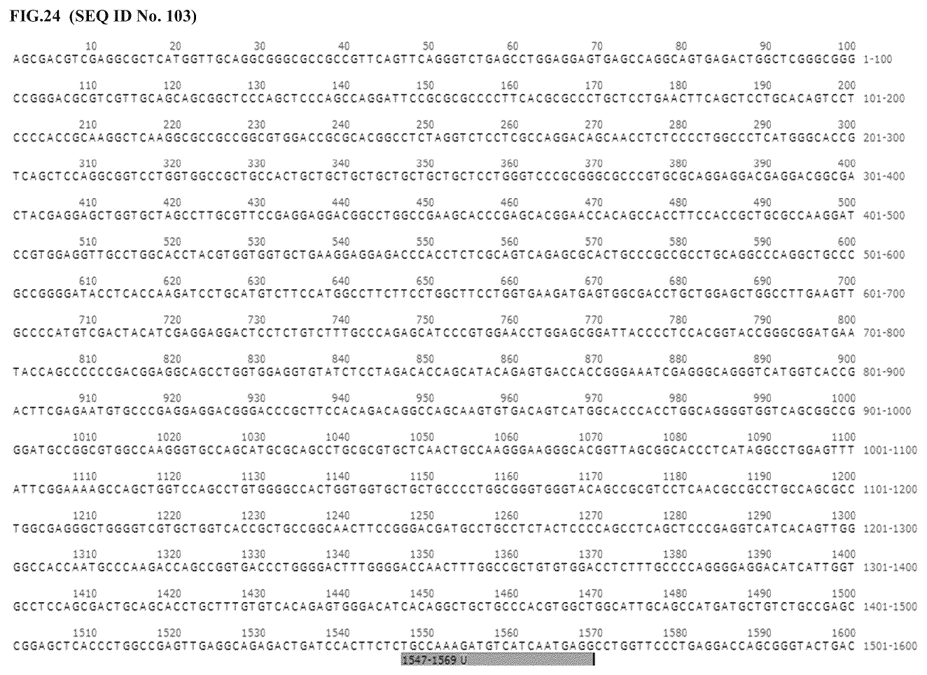

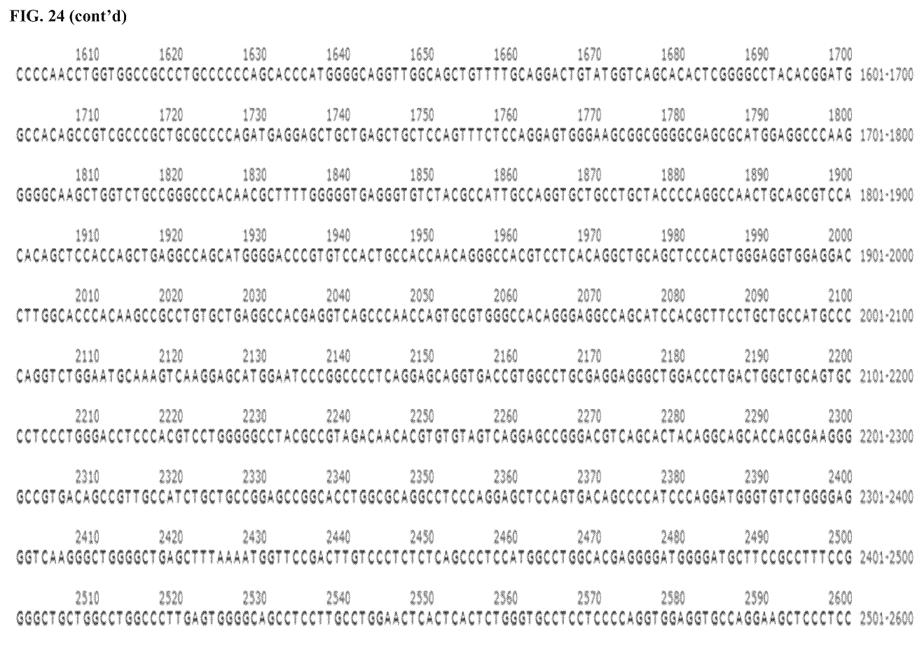

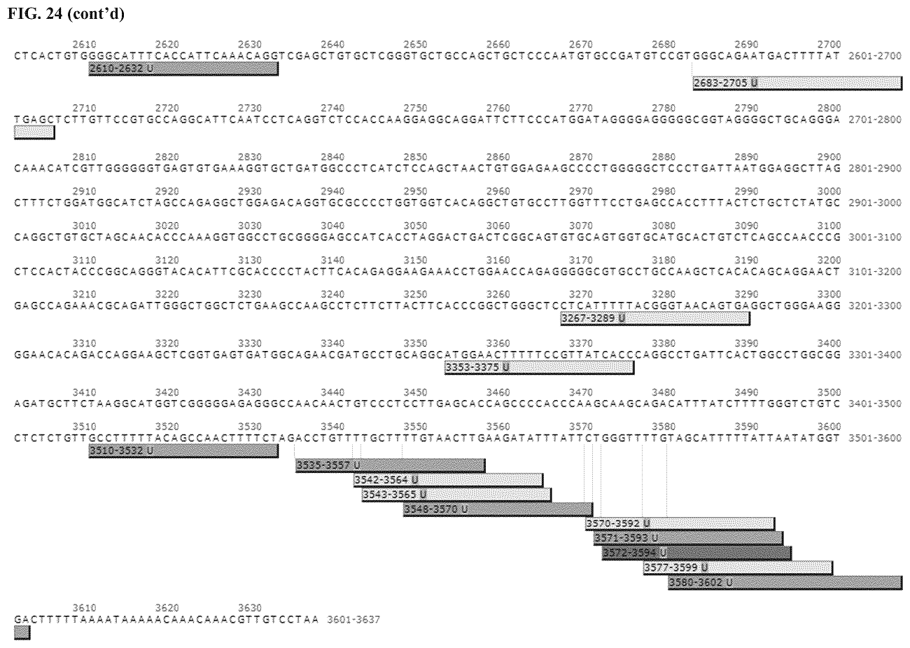

In vitro screening of the modified PCSK9 siRNA sequence. HepG2: 2×105 cells per well in 12 well plate in transfection time for 24 hr. cDNA was 30 ng in 8.2 μL. PCS siRNA concentrations in transfection: 10 nM, 1 nM, 0.1 nM, and 0.01 nM, 10× serial dilution. Lipofectamine RNAi Max 2 μL/Rxn in 98 ul OptiMEM; siRNA was applied in 100 4 of OptiMEM; then mixed together and incubate for 15 min. Multiplex PCR was HPRT probe (10 μM), 0.4 μL ). HPRT Primer used as (F+R, 20 uM each), 0.2 μL (final concentration is 200 nM). PCS probe was (10 μM, 0.4 μL ). PCS Primer (F+R, 20 uM each), 0.8 ul (final concentration is 800 nM). TaqPath 2× master mix was used as 10 μL (see FIG. 19 through FIG. 23 (b). The siRNA was further chemically modified to enhance the stabilization. The mRNA knockdown level of those modified mPCSK9 siRNA were further evaluated by serial dilution experiment. IC50 of mPCS49b is about 0.1nM-0.01 nM, IC50 of mPCS58 is about 0.1 nM, IC50 of mPCS48a is below 10 nM, IC50 of mPCS48b is about 10 nM, IC50 of mPCSd3a is about 10 pM, IC50 of mPCSd3b is below 10 nM.

### Example 12

Design of the PCSK9 siRNA sequence targeting the PCSK9 mRNA gene and minimizing off-target effects. Figure NM_174936.4 Homo sapiens proprotein convertase subtilisin/kexin type 9 (PCSK9), transcript variant 1. siDirect selects siRNAs with lower Tm value at the seed region, which contains 7 nucleotides at positions 2-8 from 5′ end of the guide strand. siRNAs down-regulate many unintended genes whose transcripts have complementarities to the siRNA seed region. The capability of siRNA to induce this seed-dependent off-target effect is highly correlated with the thermodynamic stability of the duplex formed between the seed region of the siRNA guide strand and its target mRNA. The melting temperature (Tm) for the formation of the seed duplex showed strong correlation with seed-dependent off-target effect (see Table 2).

### Example 13—In Vitro Study to Evaluate PCSK9 Conjugates in Mouse/Human Hepatocytes

PCSK9 compounds were evaluated in vitro with primary mouse or primary human hepatocytes PCSK9 samples. Cells were seeded using primary human or mouse hepatocytes using 15,000 cells per well. GalNac-conjugated duplexes were used, transfected passively using concentrations ranging between 0.064 nM to 1000 nM compounds (5-fold dilution series), antibiotic-free complete WEM medium (hepatocyte supplements, 2.5% FBS) and is incubated for 72 hours at 37 degrees C. All compounds were run on 20% TBE polyacrylamide gel to determine duplex integrity. As compounds were in the form of duplexes, no optimization of single strand concentration could be performed. Gene expression (expressed as percent of expression in non-treated (NT) cells) was measured by qPCR, adjusted to the standard curve and normalized to the reference gene, GAPDH.

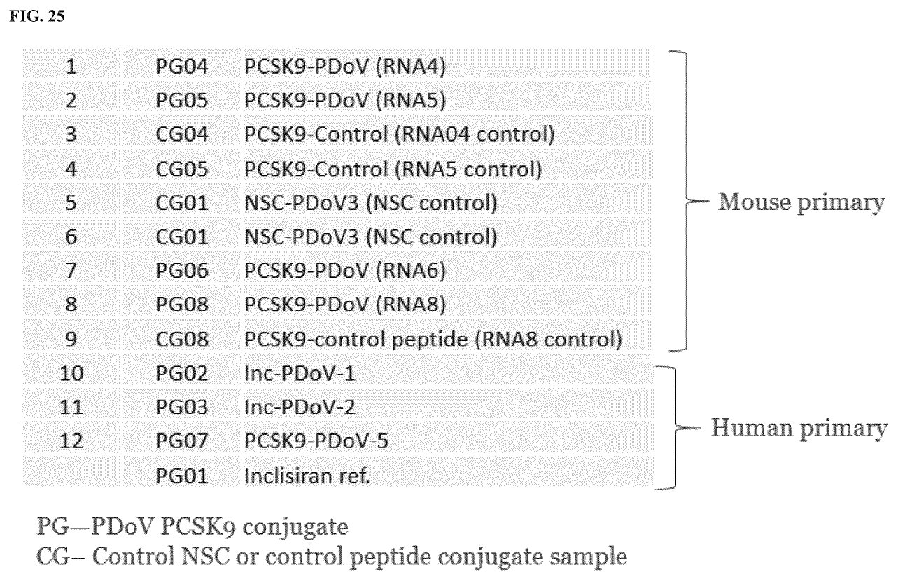

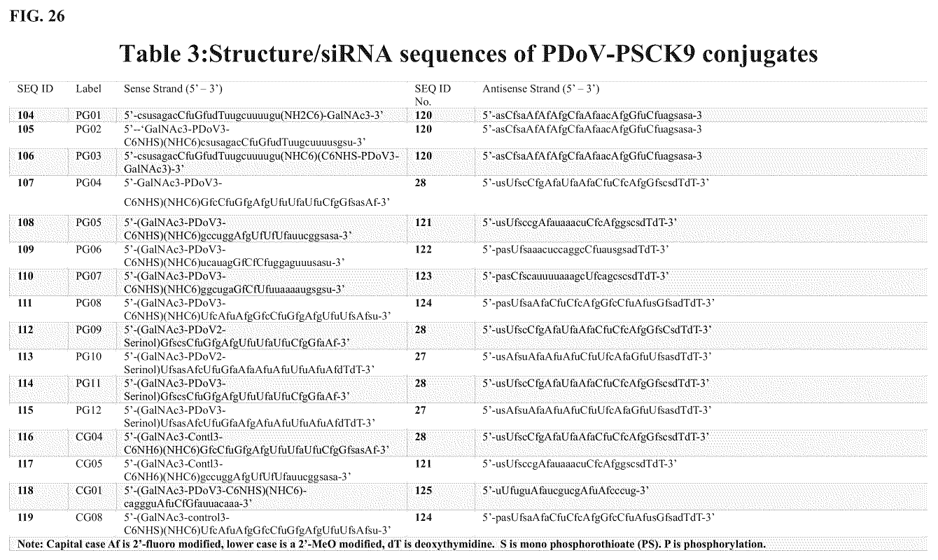

FIG. 25 shows the sample identification information for mouse and human conjugates and controls used in in vitro evaluation. FIG. 26 shows a table of the structures and siRNA sequences information for PDoV-PSCK9 conjugates used in in vitro evaluation.

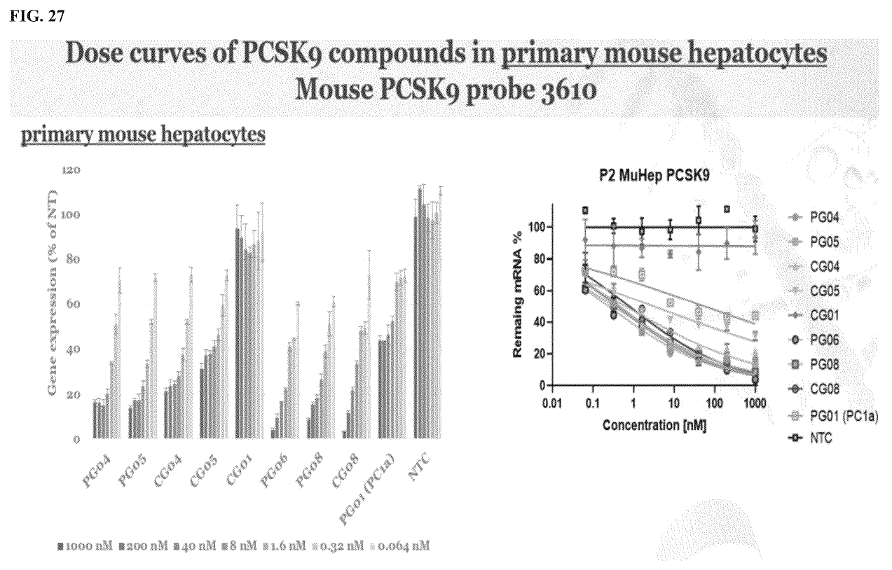

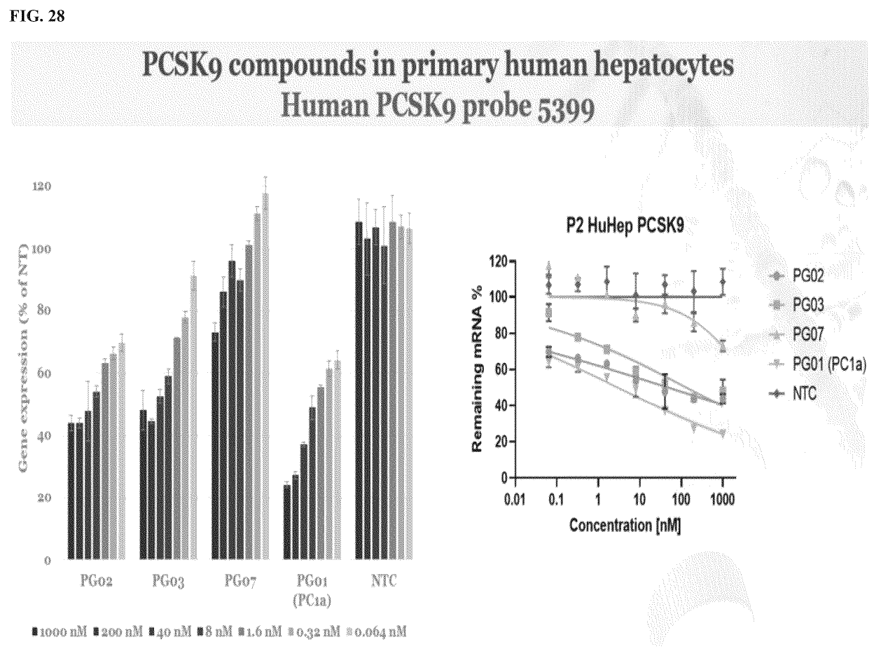

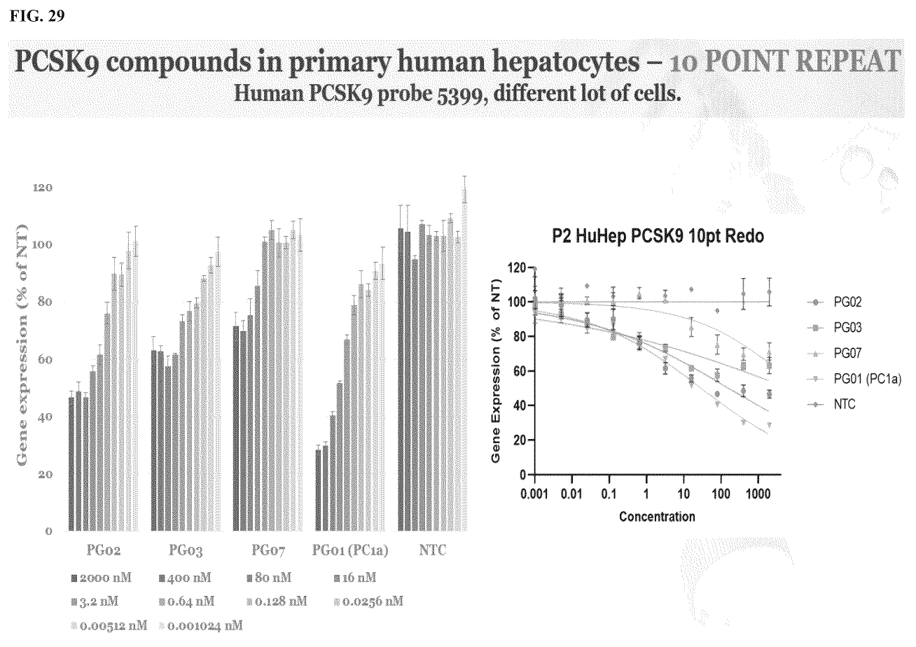

Human-derived compounds demonstrated limited activity, while mouse-derived compounds demonstrated variable activity. FIG. 27 shows dosage curves of PCSK9 compounds (PG04, PG05, PG06 and PG08, each at 7 concentrations between 0.064 nM and 1000 nM) in primary mouse hepatocytes in vitro, using the mouse PSCK9 probe 3610. Conjugates PG04 and PG05 at higher concentrations silenced PCSK9 gene expression greater than 80%, while PG06 and PG08 showed even greater ability to silence gene expression (>95%). FIG. 28 shows the results of in vitro evaluation of PCSK9 compounds (PG02, PG03, PG07, each at 7 concentrations between 0.064 nM and 1000 nM) in primary human hepatocytes using the human PCSK9 probe 5399. The positive control (inclisiran siRNA) PG01 (PC₁a) performed the best with a 75% knockdown of PCSK9 mRNA and 1000 nM. PG02 (inclisiran siRNA +PDoV-1) and PG03 (inclisiran siRNA+PDoV-2) demonstrated roughly 55% silencing; PG07 silenced only 30% at 1000 nM. In a “10-point” repetition, FIG. 29 shows the results of in vitro evaluation of PCSK9 compounds (PG02, PG03, PG07, each at a broader set of 10 concentrations ranging from 0.001024 nM through 2000 nM) in primary human hepatocytes using human PCSK9 probe 5399. The data from this repeated trial are very similar to that shown in FIG. 28.

### Example 14—In Vivo Study of PDoV-PCSK9 Conjugate PG05 from Blood and Liver Tissue Samples in Mice

mRNA expression of PCSK9 was evaluated in liver and blood tissue samples using qPCR following subcutaneous administration of a PCSK9 conjugate PG05. Four groups of C_{57/}Black6 mice (n=4 each) were administered PBS vehicle or one of three doses of PG05 (1, 3 or 10 mg/kg). Mice were sacrificed at Day 14; blood and liver biopsy samples were taken, the latter as 2° mm diameter punch biopsy samples in triplicate, one sample from each lobe: L, R, M) for isolation and analysis of RNA; samples were frozen at −80 degrees C. until use. Liver samples were thawed and centrifuged, homogenized with a pestle disruptor. A Purelink Pro 96 RNA extraction kit was used for RNA isolation. Gene expression of mouse CFB was measured by qPCR, normalized to the reference gene GAPDH, and adjusted to the standard curve. Blood samples were analyzed for PCSK9 using ELIZA. Data are expressed as a percentage of gene expression adjusted to the standard curve and normalized to the mean of PBS controls.

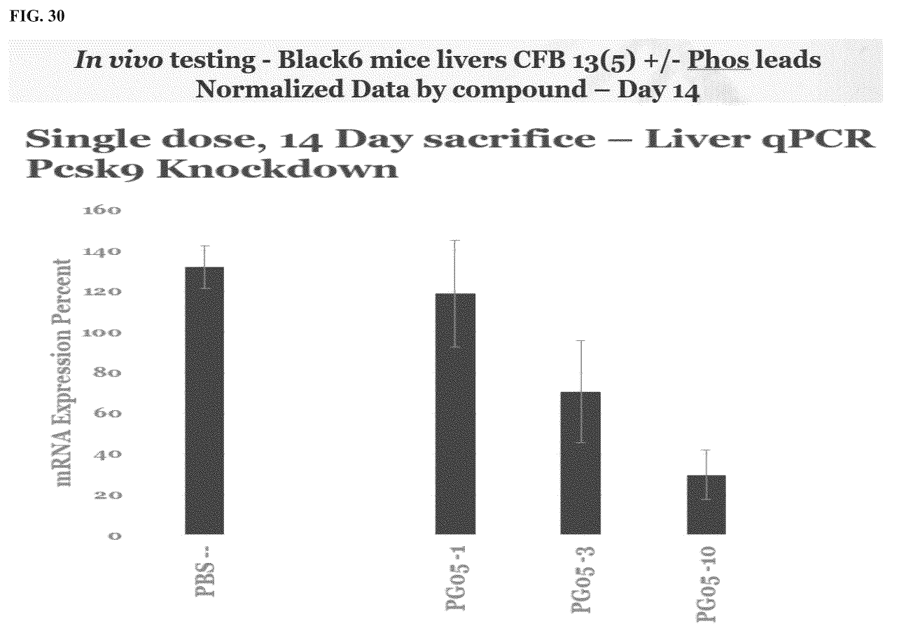

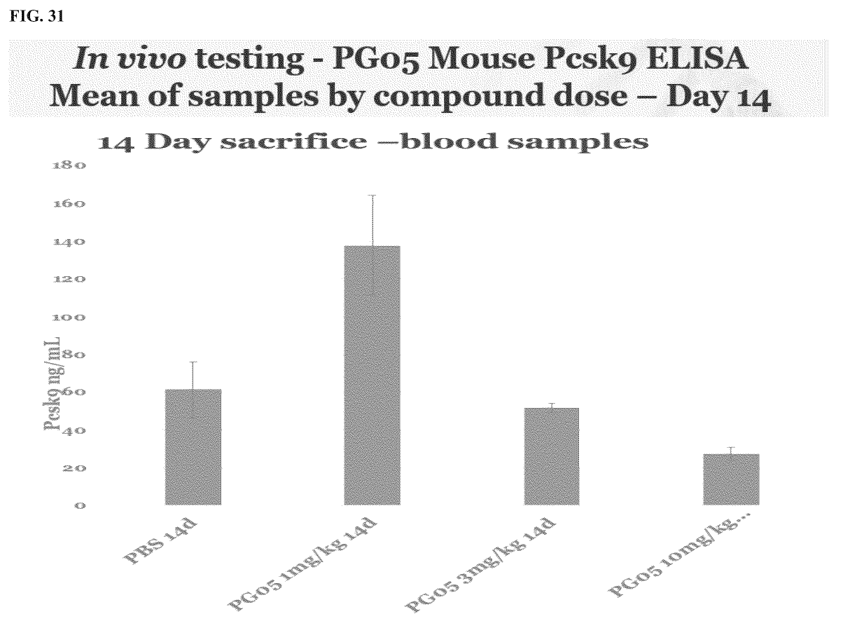

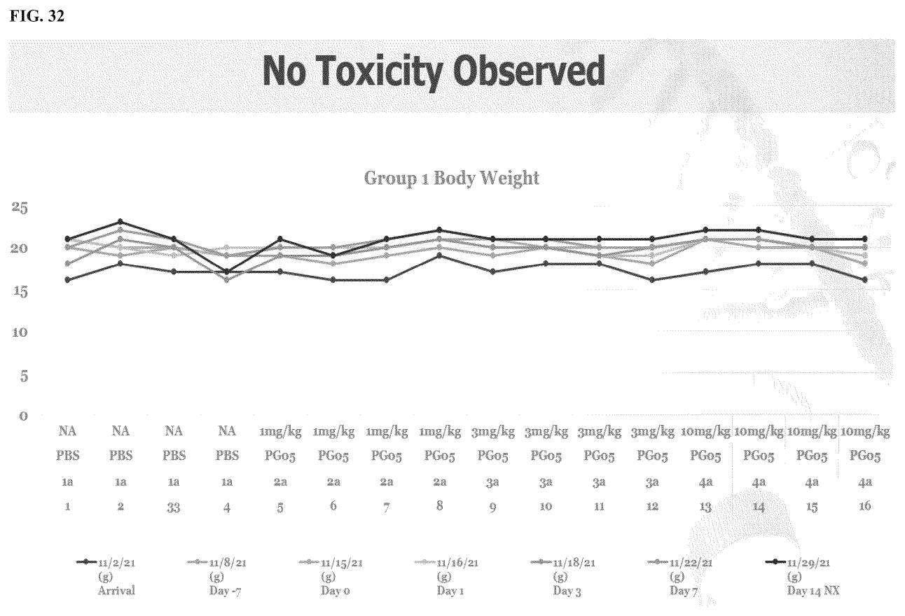

FIG. 30 shows results of in vivo evaluation of a single dose of the PCSK9 compound PG05 at 1, 3 and 10 mg/kg on Day 14 (sacrifice) in liver tissue samples of C_{57/}Black6 mice using qPCR. FIG. 31 shows the results of in vivo evaluation of PCSK9 compound PG05 at 1, 3 and 10 mg/kg on Day 14 (sacrifice) in blood samples of C_{57/}Black6 using ELISA. ELIZA data correlated well with data from qPCR analysis and a clear dose-dependent effect was observed. FIG. 32 shows that there was no toxic effect of PG05 at 1, 3 or 10 mg/kg on body weight of mice through sacrifice at Day 14.

### Example 15—In Vivo Study of PDoV-PCSK9 Conjugate PG04 from Blood and Liver Tissue Samples in Mice

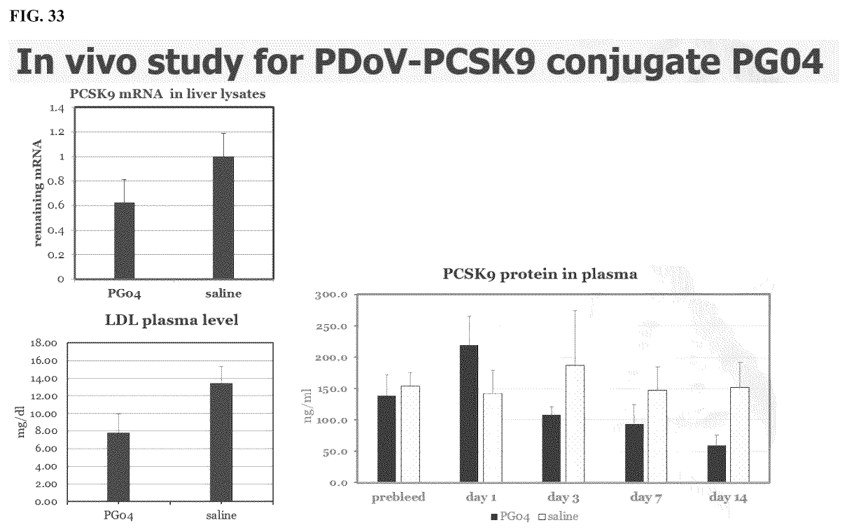

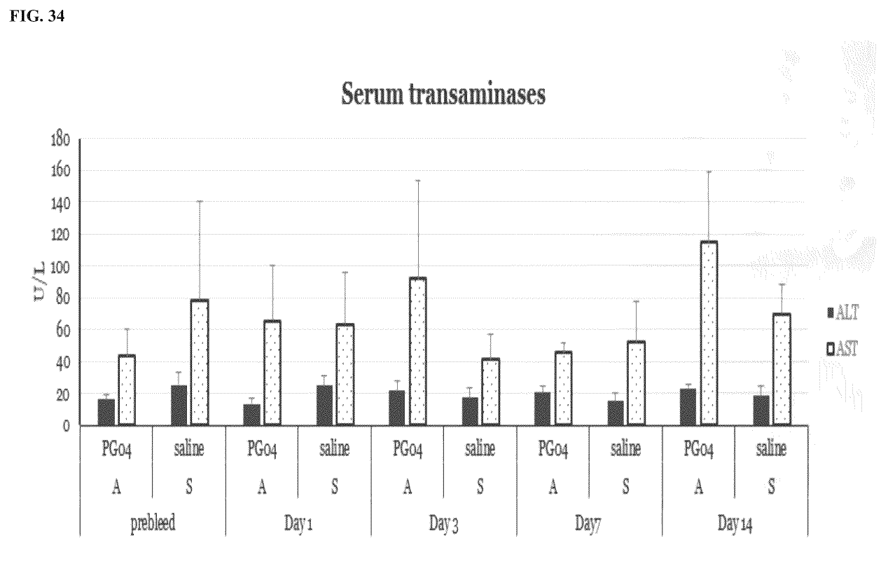

PCSK9 mRNA expression was evaluated in liver and blood tissue samples using qPCR following subcutaneous administration of a PCSK9 conjugate PG04. Four groups of C_{57/}Black6 mice (n=4 each) were administered two subcutaneous injections (10 mg/kg) at each of Day 0 and sacrificed at Day 14. Blood was drawn before administration and on Days 1, 3, 7, and 14, (day of sacrifice by heart puncture); liver biopsy samples were taken following sacrifice and snap frozen in liquid nitrogen for bDNA analysis of PCSK9 mRNA; PCSK9 was analyzed by ELIZA in plasma samples. AST, ALT and LDL cholesterol concentrations were determined in plasma. LDL cholesterol, AST and ALT in plasma were measured on a COBAS Integra 400 system samples from Day 14 blood samples (LDLc, diluted 1:2 for the assay; AST and ALT plasma diluted 1:10, both using 100 μl of the diluted sample). FIG. 33 shows the in vivo evaluation of PCSK9 mRNA in plasma and in liver lysates over 14 days, and LDL plasma levels of C_{57/}B16 mice using ELISA following twice a day subcutaneous administration of PCSK9 conjugate PG04. FIG. 34 shows serum transaminase (ALT, AST) levels in mice administered PG04 and saline prior to the start of the study and at Days 1, 3, 7 and 14.

### Example 16—In vivo Comparison of PCSK9 Conjugates in a Hyperlipidemia Mouse Model

The effect of PCSK9 conjugates on PCSK9 plasma and LDL cholesterol levels was evaluated following administration of the conjugates in a hyperlipidemic mouse model. Mice exhibiting hyperlipidemia were administered through subcutaneous injection a single dose of Alnylam's GalNAc PCSK9 conjugate (PC), or one of two different doses (1 and 3 mg/kg) of PDoV-PCSK9 conjugate PG13.

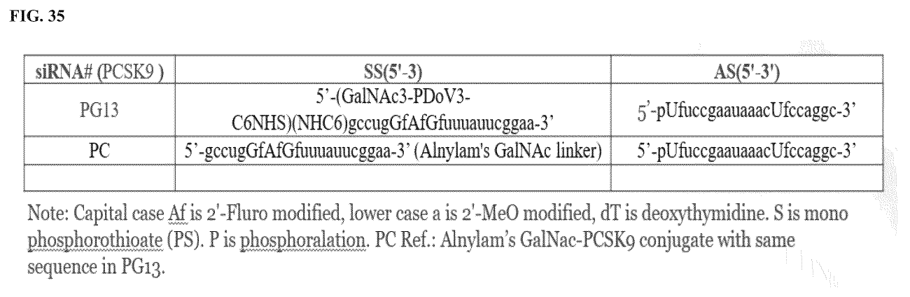

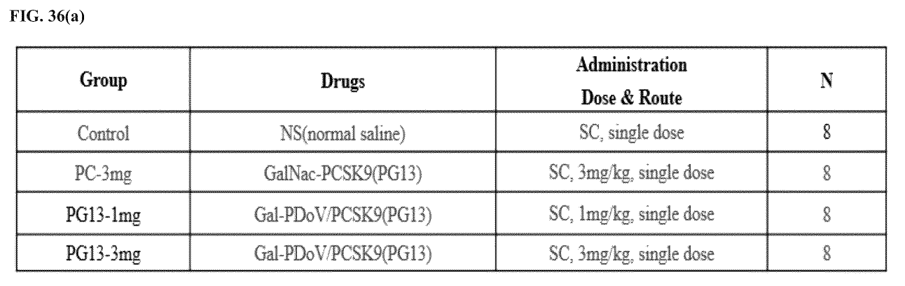

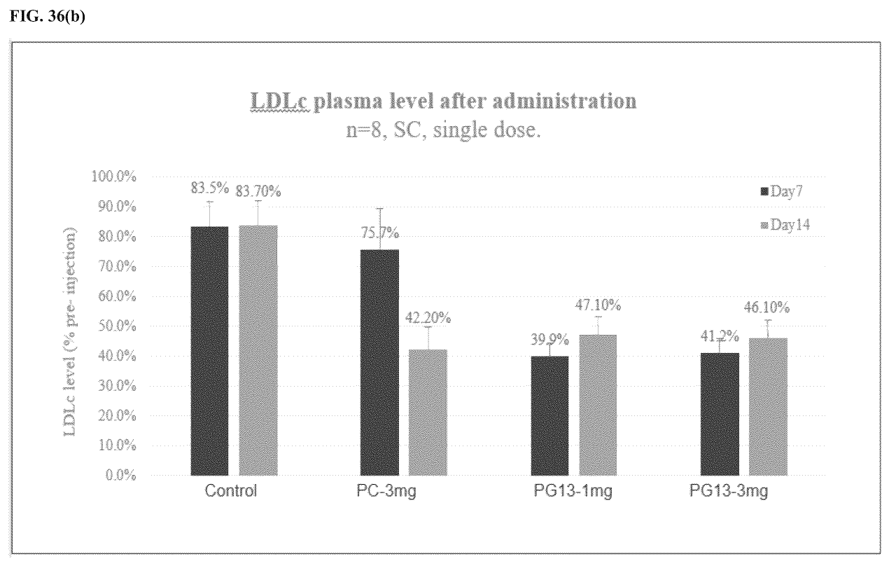

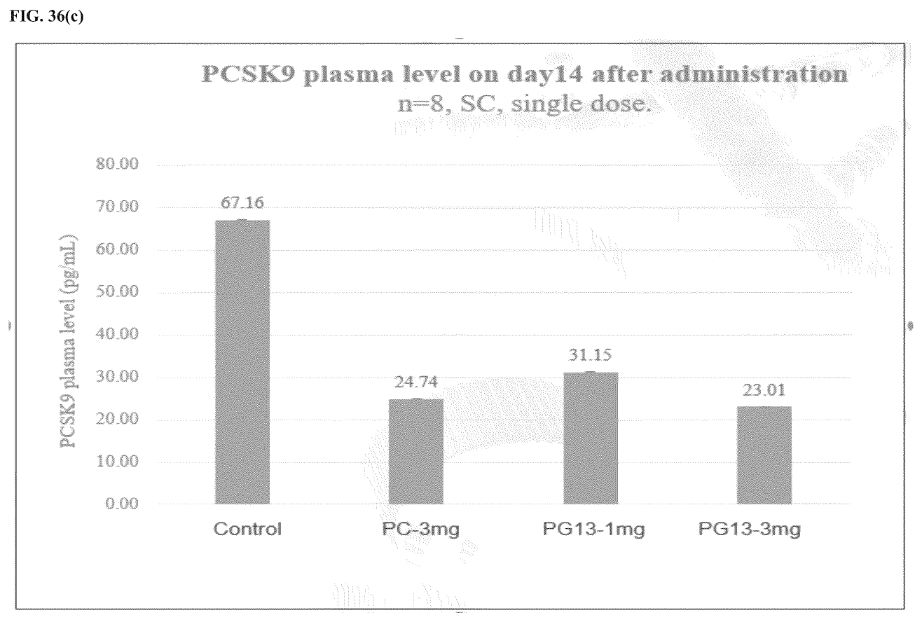

FIG. 35 is a table showing the sequence/structure of PCSK9 duplexes PG13 and PC, the latter, Alnylam's GalNAc-PCSK9 conjugate with the same sequence as PG13, used for evaluation in the vivo comparison study in a hyperlipidemia mouse model). FIG. 36 (a), (b) and (c) show the design and results of the study in a hyperlipidemic mouse model comparing the effect of GalNAc-PCSK9 conjugates in vivo. FIG. 36 (a) shows the basic study design, with the mice in each of four groups (n=8) receiving a single dose of saline (control group), PC (3 mg/kg of Alnylam's GalNAc/PCSK9 conjugate), or low (1 mg/kg) or high (3 mg/kg) doses of STP135G, Sirnaomics' GalNAc-PDoV/PCSK9 conjugate through subcutaneous administration; FIG. 36 (b) shows the mean LDLc plasma levels following administration of a single subcutaneous injection at Days 7 and 14; the data is shown as a percent of the pre-administration level. FIG. 36 (c) shows the mean PCSK9 plasma levels (pg/mL) following administration of a single subcutaneous injection at Day 14. The data demonstration that the GalNAc-PDoV PCSK9 conjugate performed similarly or superior to Alnylam's PCSK9 conjugate, PC, having the same sequence as the GalNAc-PDoV PCSK9 conjugate.

1. A chemical construct comprising a Peptide Docking Vehicle (PDoV) covalently linked to (a) a targeting moiety, and (b) a therapeutic nucleic acid, wherein said therapeutic nucleic acid inhibits expression of PCSK9 gene, and wherein the PDoV comprises multiple repeating units of histidine and lysine.

2. The construct of claim 1, wherein the targeting moiety is selected from the group consisting of N-acetyl-galactosamine (GalNAc), galactose, galactosamine, N-formal-galactosoamine, N-propionyl-galactosamine, and N-butanoylgalactosamine.

3. The construct of claim 1, wherein the therapeutic nucleic acid comprises an siRNA, an antisense oligonucleotide, a miRNA, an aptamer, a decoy oligonucleotide, or a CpG motif.

4. The construct of claim 3, wherein said therapeutic nucleic acid is an siRNA selected from the group consisting of SEQ ID Nos. 13-102.

5. The construct of claim 1, wherein the PDoV construct comprises an endosomal release motif that comprises at least two targeting moieties and/or at least one therapeutic oligonucleotide.

6. The construct of claim 5, wherein said PDoV has the structure I or II, wherein A and B are independently a peptide sequence of H, K, R, HH, HHH, HHRH (SEQ ID NO: 1), HHK, HHHK (SEQ ID NO: 2), D is an siRNA, R_{L} is a targeting ligand, and R_{S} is a covalent linker to the nucleic acid, 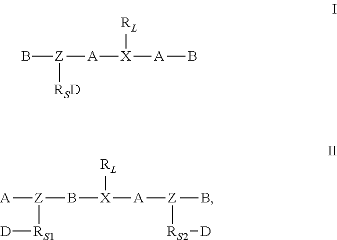
wherein R_{S} is a bioorthogonal reactive moiety that links the nucleic acid to said PDoV peptide, wherein the reactive moiety is selected from the group consisting of an amine, hydrazine, N-hydroxysuccinimide, azido, alkyne, carboxylic acid, thiol, maleimide, and phosphine diester, and wherein said siRNA molecule comprises a duplex of two complimentary, single-stranded oligonucleotides, wherein the oligonucleotides are the same length, and each has a length of 19-27 bases.

7. The construct of claim 1, wherein the PDoV peptide construct has a structure selected from the group consisting of PDoV 1 (SEQ ID NO: 130), PDoV 2 (SEQ ID NO: 5), PDoV 3 (SEQ ID NO: 131), PDoV 3a (SEQ ID NO: 132), and PDoV 4 (SEQ ID NO: 133): 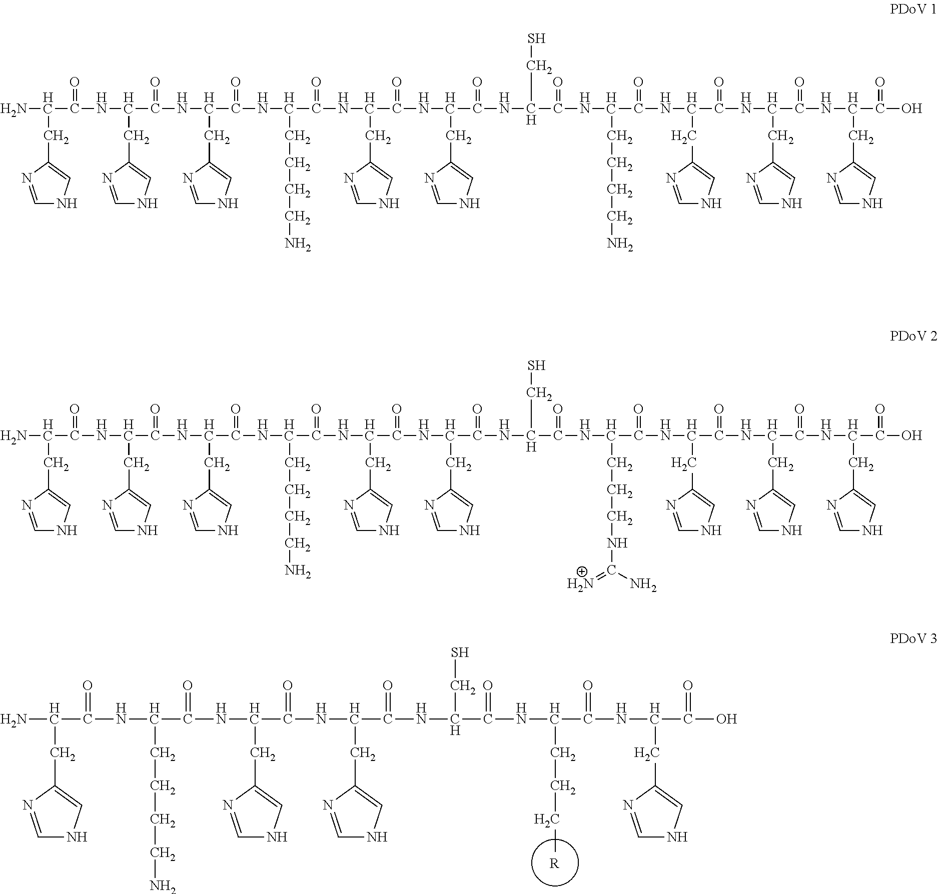 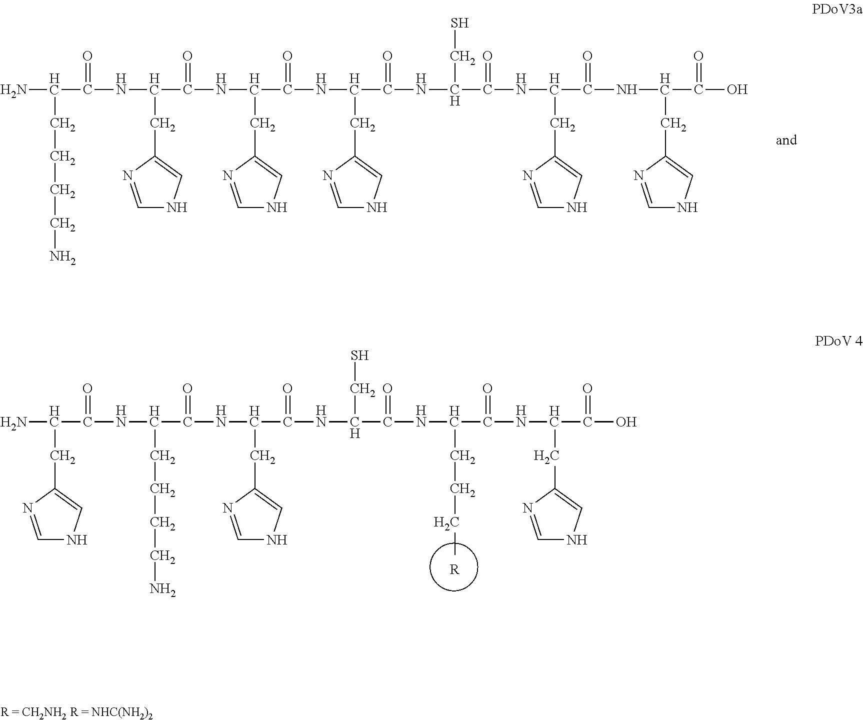
wherein the linker between the targeting ligand and the PDoV peptide optionally comprises a polyethylene glycol chain —(CH₂CH₂O)ₙ—, or an alkylene chain —(CH₂CH₂)ₙ— chain, wherein n is an integer from 2-15.

8. The construct of claim 1, wherein said targeting moiety comprises a ligand covalently linked to said PDoV via a linker of formula III or IV: 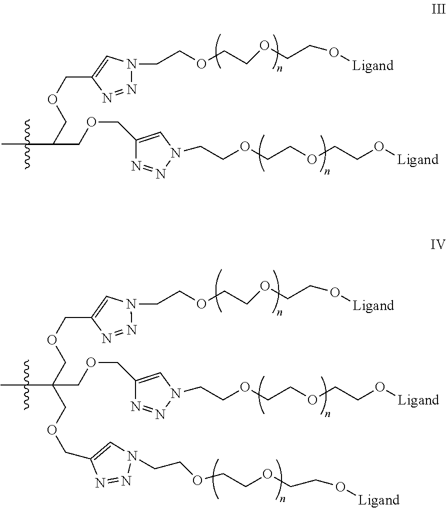
wherein n is 1, 2, or 3.

9. The construct of claim 2, wherein the therapeutic nucleic acid comprises at least one nucleotide chemically modified at the 2′ position, wherein the chemically modified nucleotide is selected from the group consisting of 2′-O-methyl, 2′-fluoro, 2′-O-methoxyethyl and 2′-O-allyl:

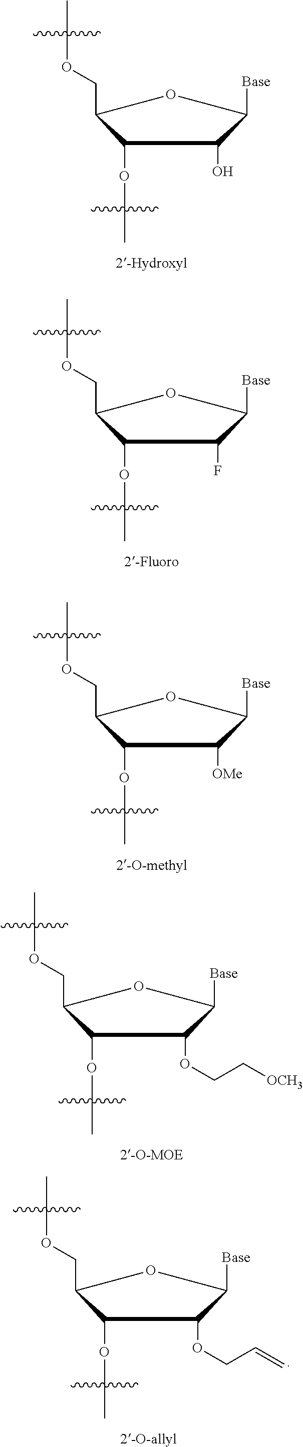

10. The construct of claim 3, wherein said therapeutic nucleic acid is an siRNA molecule comprising one or more chemically modified nucleotides selected from the group consisting of a phosphorothioate diester or phosphorodithioate diester.

11. The construct of claim 1, wherein the therapeutic nucleic acid comprises a first siRNA and a second siRNA, wherein each of the first and the second siRNAs targets the PCSK9 gene and wherein each siRNA is selected from the group consisting of SEQ ID Nos. 13-102.

12. The construct of claim 1, wherein the therapeutic nucleic acid is covalently linked by a linker to said PDoV via the 5′ or 3′ position of a nucleotide or nucleoside in said nucleic acid, and wherein the linker is an aliphatic chain, a polyethylene glycol chain, a hydrophilic chain or a hydrophobic chain.

13. The construct of claim 1, wherein the targeting moiety is N-acetyl-galactosamine (GalNAc).

14. A construct according to claim 1 having a structure (SEQ ID NOS 134-135, respectively, in order of appearance):

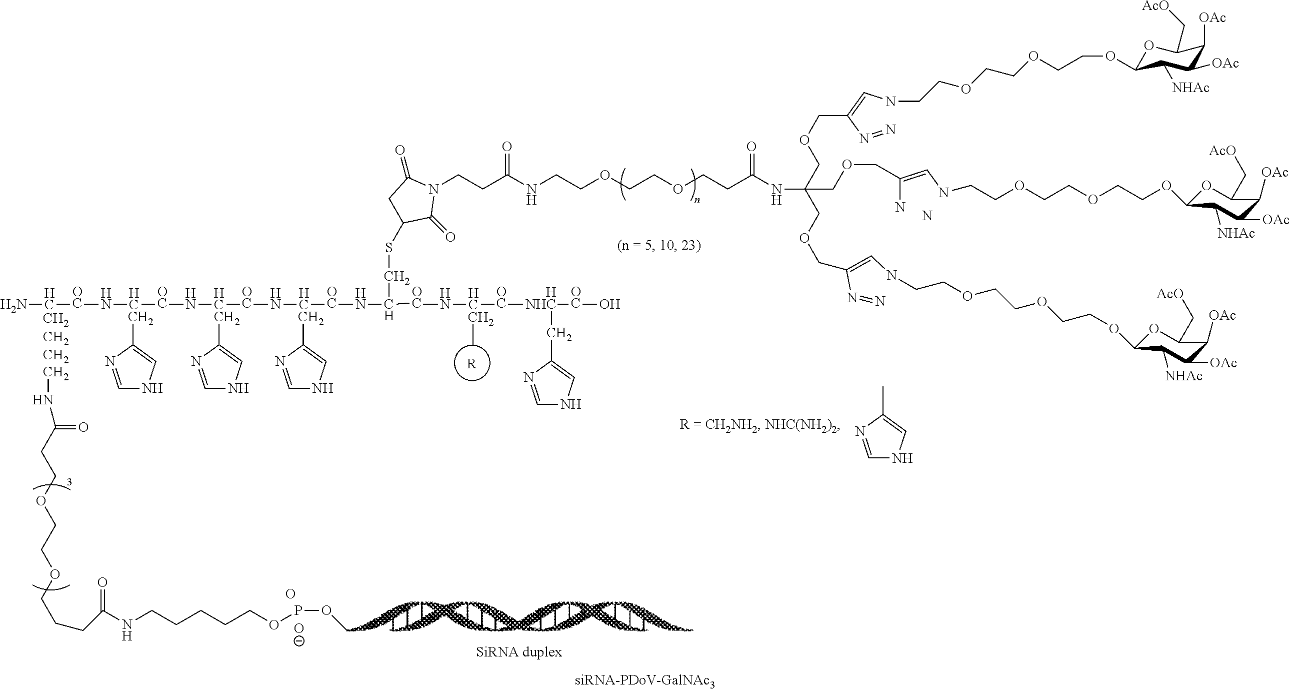

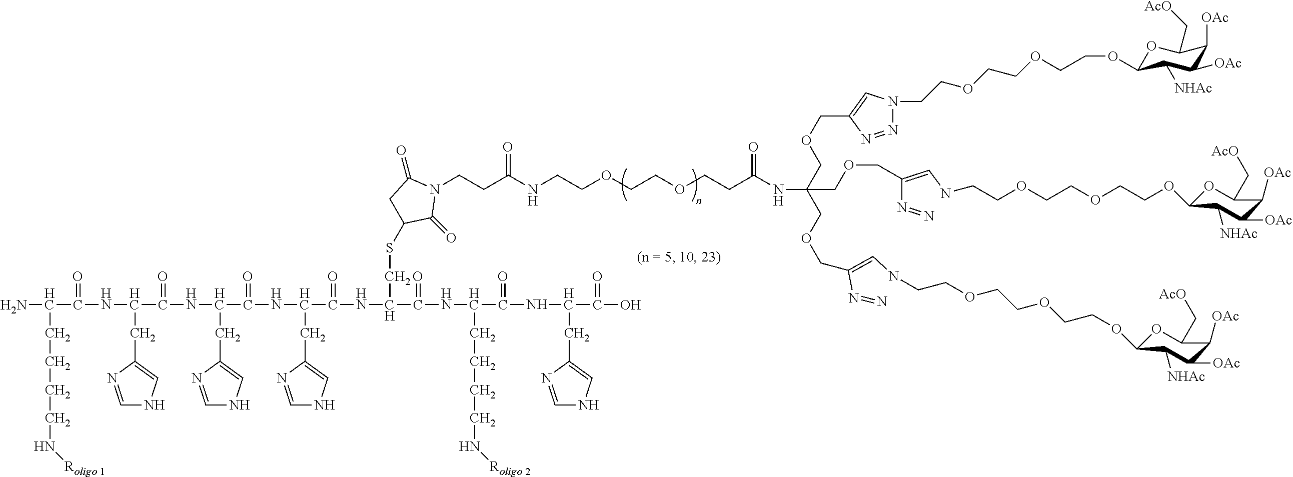

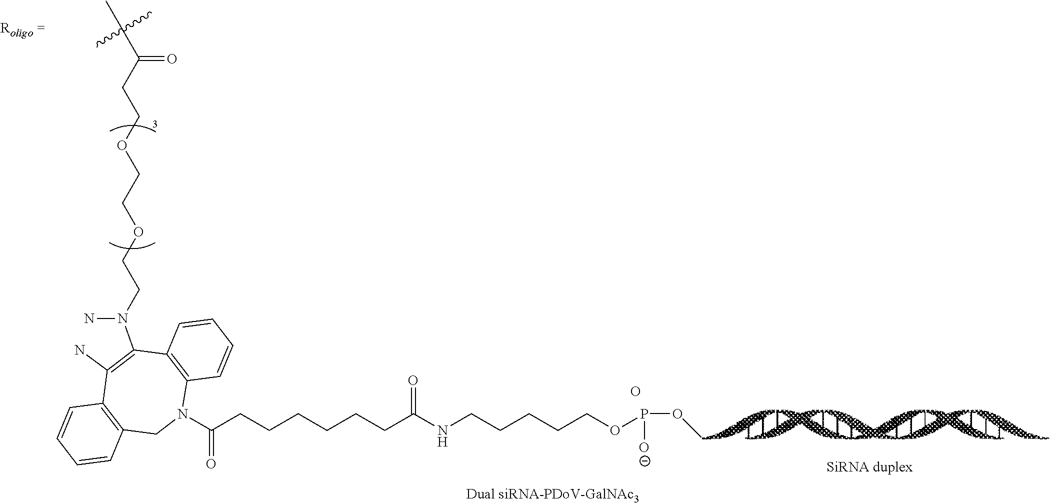

15. A pharmaceutical composition comprising a construct according to claim 1 and a pharmaceutically acceptable carrier.

16. A method of lowering the serum LDL cholesterol or treating PCSK9 gene related cancer in a subject, comprising administering to the described subject a pharmaceutical composition according to claim 15.

17. The method of claim 16, wherein said subject is a primate.

18. The method of claim 16, wherein said subject is a human.


SEQUENCE LISTING (144 sequences)

SEQ ID NO: 1 - protein, 4 aa, synthetic construct
  REGION 1..4: note = Description of Artificial Sequence: Synthetic peptide
HHHH

SEQ ID NO: 2 - protein, 4 aa, synthetic construct
  REGION 1..4: note = Description of Artificial Sequence: Synthetic peptide
HHHK

SEQ ID NO: 3 - protein, 210 aa, synthetic construct
  REGION 1..210: note = Description of Artificial Sequence: Synthetic polypeptide
  VARIANT 1..10: note = SITE - This region may encompass 1-10 repeating residues
  VARIANT 11..20: note = SITE - This region may encompass 1-10 repeating residues
  VARIANT 21..30: note = SITE - This region may encompass 1-10 repeating residues
  VARIANT 31..40: note = SITE - This region may encompass 1-10 repeating residues
  VARIANT 41..50: note = SITE - This region may encompass 1-10 repeating residues
  VARIANT 51..60: note = SITE - This region may encompass 1-10 repeating residues
  VARIANT 61..70: note = SITE - This region may encompass 1-10 repeating residues
  VARIANT 71..80: note = SITE - This region may encompass 1-10 repeating residues
  VARIANT 81..90: note = SITE - This region may encompass 1-10 repeating residues
  VARIANT 91..100: note = SITE - This region may encompass 1-10 repeating residues
  VARIANT 101..110: note = SITE - This region may encompass 1-10 repeating residues
  VARIANT 111..120: note = SITE - This region may encompass 1-10 repeating residues
  VARIANT 121..130: note = SITE - This region may encompass 1-10 repeating residues
  VARIANT 131..140: note = SITE - This region may encompass 1-10 repeating residues
  VARIANT 141..150: note = SITE - This region may encompass 1-10 repeating residues
  VARIANT 151..160: note = SITE - This region may encompass 1-10 repeating residues
  VARIANT 161..170: note = SITE - This region may encompass 1-10 repeating residues
  VARIANT 171..180: note = SITE - This region may encompass 1-10 repeating residues
  VARIANT 181..190: note = SITE - This region may encompass 1-10 repeating residues
  VARIANT 191..200: note = SITE - This region may encompass 1-10 repeating residues
  VARIANT 1..200: note = SITE - This region may encompass 1-10 "(H)n(K)m" units, wherein n = 1-10 and m = 1-10
  VARIANT 201..205: note = SITE - This region may encompass 1-5 repeating residues or be absent in its entirety
  VARIANT 201..210: note = MOD_RES - Any amino acid or absent
  VARIANT 206..210: note = SITE - This region may encompass 1-5 repeating residues or be absent in its entirety
  REGION 1..210: note = See specification as filed for detailed description of substitutions and preferred embodiments
HHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKXXXXXXXXXX

SEQ ID NO: 4 - protein, 11 aa, synthetic construct
  REGION 1..11: note = Description of Artificial Sequence: Synthetic peptide
  SITE 4: note = MOD_RES - Lys(PEG4-N3) - polyethylene glycol 4-azide attached to side chain of Lysine
HHHKHHCKHHH

SEQ ID NO: 5 - protein, 11 aa, synthetic construct
  REGION 1..11: note = Description of Artificial Sequence: Synthetic peptide
HHHKHHCRHHH

SEQ ID NO: 6 - protein, 7 aa, synthetic construct
  REGION 1..7: note = Description of Artificial Sequence: Synthetic peptide
  SITE 1: note = MOD_RES - Lys(PEG4-N3) - polyethylene glycol 4-azide attached to side chain of Lysine
KHHHCHH

SEQ ID NO: 7 - protein, 11 aa, synthetic construct
  REGION 1..11: note = Description of Artificial Sequence: Synthetic peptide
  SITE 4: note = MOD_RES - Lys(PEG4-N3) - polyethylene glycol 4-azide attached to side chain of Lysine
HHHKHHCKHHH

SEQ ID NO: 8 - protein, 12 aa, synthetic construct
  REGION 1..12: note = Description of Artificial Sequence: Synthetic peptide
  SITE 4: note = MOD_RES - Lys(PEG4-N3) - polyethylene glycol 4-azide attached to side chain of Lysine
  SITE 8: note = polyethylene glycol 6-N-acetylgalactosamine x3 attached to side chain of Cysteine
HHHKHHHCKHHH

SEQ ID NO: 9 - protein, 7 aa, synthetic construct
  REGION 1..7: note = Description of Artificial Sequence: Synthetic peptide
  SITE 1: note = MOD_RES - Lys(PEG4-N3) - polyethylene glycol 4-azide attached to side chain of Lysine
KSSSCSS

SEQ ID NO: 10 - protein, 11 aa, synthetic construct
  REGION 1..11: note = Description of Artificial Sequence: Synthetic peptide
  SITE 4: note = MOD_RES - Lys(PEG4-N3) - polyethylene glycol 4-azide-Peptide docking vehicle 1-N-acetylgalactosamine x3 attached to side chain of Lysine
  SITE 8: note = polyethylene glycol 6-N-acetylgalactosamine x3 attached to side chain of Cysteine
HHHKHHHCHHH

SEQ ID NO: 11 - protein, 16 aa, synthetic construct
  REGION 1..16: note = Description of Artificial Sequence: Synthetic peptide
  VARIANT 4: note = MOD_RES - Any amino acid
  VARIANT 9: note = MOD_RES - Any amino acid
  VARIANT 13: note = MOD_RES - Any amino acid
HHHXHHHKXHHHXHHH

SEQ ID NO: 12 - protein, 16 aa, synthetic construct
  REGION 1..16: note = Description of Artificial Sequence: Synthetic peptide
  VARIANT 4..5: note = MOD_RES - Any amino acid
  VARIANT 9: note = MOD_RES - Any amino acid
  VARIANT 12..13: note = MOD_RES - Any amino acid
HHHXXHHKXHHXXHHH

SEQ ID NO: 13 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttcttcaagttacaaaagctt

SEQ ID NO: 14 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaagttacaaaagcaaaatt

SEQ ID NO: 15 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcaaaacaggtctagaaaatt

SEQ ID NO: 16 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tataaatatcttcaagttatt

SEQ ID NO: 17 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttacaaaagcaaaacaggtt

SEQ ID NO: 18 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtgacacaaagcaggtgctt

SEQ ID NO: 19 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttcaagttacaaaagcaatt

SEQ ID NO: 20 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
gggagtatccggacctcaatt

SEQ ID NO: 21 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ggagtatccggacctcaaatt

SEQ ID NO: 22 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ttccgaataaactccaggctt

SEQ ID NO: 23 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
tgtgacacaaagcaggtgctt

SEQ ID NO: 24 - DNA, 22 bp, synthetic construct
  misc_feature 1..22: note = Description of Artificial Sequence: Synthetic oligonucleotide
  source 1..22: mol_type = other DNA
  modified_base 1: mod_base = OTHER; note = 2'-fluoro modified nucleotide
  modified_base 2: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-fluoro modified nucleotide
  misc_feature 1..20: note = RNA
  misc_feature 21..22: note = DNA
tgtggacacaaagcaggtgctt

SEQ ID NO: 25 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
ttccgaataaactccaggctt

SEQ ID NO: 26 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-fluoro modified nucleotide
  modified_base 2: mod_base = OTHER; note = 2'-fluoro modified nucleotide
  modified_base 3: mod_base = OTHER; note = 2'-fluoro modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-fluoro modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-fluoro modified nucleotide
  misc_feature 1..19: note = RNA
  misc_feature 20..21: note = DNA
ttccgaataaactccaggctt

SEQ ID NO: 27 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tataaatatcttcaagttatt

SEQ ID NO: 28 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ttccgaataaactccaggctt

SEQ ID NO: 29 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
ttcttcaagttacaaaagctt

SEQ ID NO: 30 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ttcttcaagttacaaaagctt

SEQ ID NO: 31 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
tcaagttacaaaagcaaaatt

SEQ ID NO: 32 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tcaagttacaaaagcaaaatt

SEQ ID NO: 33 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
tcaaaacaggtctagaaaatt

SEQ ID NO: 34 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tcaaaacaggtctagaaaatt

SEQ ID NO: 35 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
tttacaaaagcaaaacaggtt

SEQ ID NO: 36 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttacaaaagcaaaacaggtt

SEQ ID NO: 37 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
tttcaagttacaaaagaaatt

SEQ ID NO: 38 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
tttcaagttacaaaagaaatt

SEQ ID NO: 39 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
gggagtatccggacctcaatt

SEQ ID NO: 40 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
gggagtatccggacctcaatt

SEQ ID NO: 41 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  misc_feature 1..19: note = RNA
  misc_feature 20..21: note = DNA
ggagtatccggacctcaaatt

SEQ ID NO: 42 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ggagtatccggacctcaaatt

SEQ ID NO: 43 - RNA, 19 nt, synthetic construct
  misc_feature 1..19: note = Description of Artificial Sequence: Synthetic oligonucleotide
  source 1..19: mol_type = other RNA
gcttttgtaacttgaagaa

SEQ ID NO: 44 - RNA, 19 nt, synthetic construct
  misc_feature 1..19: note = Description of Artificial Sequence: Synthetic oligonucleotide
  source 1..19: mol_type = other RNA
ttttgcttttgtaacttga

SEQ ID NO: 45 - RNA, 19 nt, synthetic construct
  misc_feature 1..19: note = Description of Artificial Sequence: Synthetic oligonucleotide
  source 1..19: mol_type = other RNA
ttttctagacctgttttga

SEQ ID NO: 46 - RNA, 19 nt, synthetic construct
  misc_feature 1..19: note = Description of Artificial Sequence: Synthetic oligonucleotide
  source 1..19: mol_type = other RNA
taacttgaagatatttata

SEQ ID NO: 47 - RNA, 19 nt, synthetic construct
  misc_feature 1..19: note = Description of Artificial Sequence: Synthetic oligonucleotide
  source 1..19: mol_type = other RNA
cctgttttgcttttgtaaa

SEQ ID NO: 48 - RNA, 19 nt, synthetic construct
  misc_feature 1..19: note = Description of Artificial Sequence: Synthetic oligonucleotide
  source 1..19: mol_type = other RNA
gcacctgctttgtgtcaca

SEQ ID NO: 49 - RNA, 19 nt, synthetic construct
  misc_feature 1..19: note = Description of Artificial Sequence: Synthetic oligonucleotide
  source 1..19: mol_type = other RNA
ttgcttttgtaacttgaaa

SEQ ID NO: 50 - RNA, 19 nt, synthetic construct
  misc_feature 1..19: note = Description of Artificial Sequence: Synthetic oligonucleotide
  source 1..19: mol_type = other RNA
ccctcataggcctggagtt

SEQ ID NO: 51 - RNA, 19 nt, synthetic construct
  misc_feature 1..19: note = Description of Artificial Sequence: Synthetic oligonucleotide
  source 1..19: mol_type = other RNA
cctcataggcctggagttt

SEQ ID NO: 52 - RNA, 19 nt, synthetic construct
  misc_feature 1..19: note = Description of Artificial Sequence: Synthetic oligonucleotide
  source 1..19: mol_type = other RNA
gcctggagtttattcggaa

SEQ ID NO: 53 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
gcacctgctttgtgtcaca

SEQ ID NO: 54 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
gcacctgctttgtgtcaca

SEQ ID NO: 55 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
gcctggagtttattcggaa

SEQ ID NO: 56 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-fluoro modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
gcctggagtttattcggaa

SEQ ID NO: 57 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  misc_feature 1..19: note = RNA
  misc_feature 20..21: note = DNA
taacttgaagatatttatatt

SEQ ID NO: 58 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
gcctggagtttattcggaa

SEQ ID NO: 59 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
gcttttgtaacttgaagaa

SEQ ID NO: 60 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
gcttttgtaacttgaagaa

SEQ ID NO: 61 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
ttttgcttttgtaacttga

SEQ ID NO: 62 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
ttttgcttttgtaacttga

SEQ ID NO: 63 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
ttttctagacctgttttga

SEQ ID NO: 64 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
ttttctagacctgttttga

SEQ ID NO: 65 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
cctgttttgcttttgtaaa

SEQ ID NO: 66 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
cctgttttgcttttgtaaa

SEQ ID NO: 67 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
ttgcttttgtaacttgaaa

SEQ ID NO: 68 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
ttgcttttgtaacttgaaa

SEQ ID NO: 69 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
ccctcataggcctggagtt

SEQ ID NO: 70 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
ccctcataggcctggagtt

SEQ ID NO: 71 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
cctcataggcctggagttt

SEQ ID NO: 72 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
cctcataggcctggagttt

SEQ ID NO: 73 - RNA, 21 nt, synthetic construct
  misc_feature 1..21: note = Description of Artificial Sequence: Synthetic oligonucleotide
  source 1..21: mol_type = other RNA
tcattgatgacatctttggca

SEQ ID NO: 74 - RNA, 21 nt, synthetic construct
  misc_feature 1..21: note = Description of Artificial Sequence: Synthetic oligonucleotide
  source 1..21: mol_type = other RNA
tgtttgaatggtgaaatgccc

SEQ ID NO: 75 - RNA, 21 nt, synthetic construct
  misc_feature 1..21: note = Description of Artificial Sequence: Synthetic oligonucleotide
  source 1..21: mol_type = other RNA
tcaataaaagtcattctgccc

SEQ ID NO: 76 - RNA, 21 nt, synthetic construct
  misc_feature 1..21: note = Description of Artificial Sequence: Synthetic oligonucleotide
  source 1..21: mol_type = other RNA
actgttacccgtaaaaatgag

SEQ ID NO: 77 - RNA, 21 nt, synthetic construct
  misc_feature 1..21: note = Description of Artificial Sequence: Synthetic oligonucleotide
  source 1..21: mol_type = other RNA
tgataacggaaaaagttccat

SEQ ID NO: 78 - RNA, 21 nt, synthetic construct
  misc_feature 1..21: note = Description of Artificial Sequence: Synthetic oligonucleotide
  source 1..21: mol_type = other RNA
aaaagttggctgtaaaaaggc

SEQ ID NO: 79 - RNA, 21 nt, synthetic construct
  misc_feature 1..21: note = Description of Artificial Sequence: Synthetic oligonucleotide
  source 1..21: mol_type = other RNA
agttacaaaagcaaaacaggt

SEQ ID NO: 80 - RNA, 21 nt, synthetic construct
  misc_feature 1..21: note = Description of Artificial Sequence: Synthetic oligonucleotide
  source 1..21: mol_type = other RNA
atcttcaagttacaaaagcaa

SEQ ID NO: 81 - RNA, 21 nt, synthetic construct
  misc_feature 1..21: note = Description of Artificial Sequence: Synthetic oligonucleotide
  source 1..21: mol_type = other RNA
tatcttcaagttacaaaagca

SEQ ID NO: 82 - RNA, 21 nt, synthetic construct
  misc_feature 1..21: note = Description of Artificial Sequence: Synthetic oligonucleotide
  source 1..21: mol_type = other RNA
ataaatatcttcaagttacaa

SEQ ID NO: 83 - RNA, 21 nt, synthetic construct
  misc_feature 1..21: note = Description of Artificial Sequence: Synthetic oligonucleotide
  source 1..21: mol_type = other RNA
taaaaatgctacaaaacccag

SEQ ID NO: 84 - RNA, 21 nt, synthetic construct
  misc_feature 1..21: note = Description of Artificial Sequence: Synthetic oligonucleotide
  source 1..21: mol_type = other RNA
ataaaaatgctacaaaaccca

SEQ ID NO: 85 - RNA, 21 nt, synthetic construct
  misc_feature 1..21: note = Description of Artificial Sequence: Synthetic oligonucleotide
  source 1..21: mol_type = other RNA
aataaaaatgctacaaaaccc

SEQ ID NO: 86 - RNA, 21 nt, synthetic construct
  misc_feature 1..21: note = Description of Artificial Sequence: Synthetic oligonucleotide
  source 1..21: mol_type = other RNA
atattaataaaaatgctacaa

SEQ ID NO: 87 - RNA, 21 nt, synthetic construct
  misc_feature 1..21: note = Description of Artificial Sequence: Synthetic oligonucleotide
  source 1..21: mol_type = other RNA
accatattaataaaaatgcta

SEQ ID NO: 88 - RNA, 21 nt, synthetic construct
  misc_feature 1..21: note = Description of Artificial Sequence: Synthetic oligonucleotide
  source 1..21: mol_type = other RNA
ccaaagatgtcatcaatgagg

SEQ ID NO: 89 - RNA, 21 nt, synthetic construct
  misc_feature 1..21: note = Description of Artificial Sequence: Synthetic oligonucleotide
  source 1..21: mol_type = other RNA
gcatttcaccattcaaacagg

SEQ ID NO: 90 - RNA, 21 nt, synthetic construct
  misc_feature 1..21: note = Description of Artificial Sequence: Synthetic oligonucleotide
  source 1..21: mol_type = other RNA
gcagaatgacttttattgagc

SEQ ID NO: 91 - RNA, 21 nt, synthetic construct
  misc_feature 1..21: note = Description of Artificial Sequence: Synthetic oligonucleotide
  source 1..21: mol_type = other RNA
catttttacgggtaacagtga

SEQ ID NO: 92 - RNA, 21 nt, synthetic construct
  misc_feature 1..21: note = Description of Artificial Sequence: Synthetic oligonucleotide
  source 1..21: mol_type = other RNA
ggaactttttccgttatcacc

SEQ ID NO: 93 - RNA, 21 nt, synthetic construct
  misc_feature 1..21: note = Description of Artificial Sequence: Synthetic oligonucleotide
  source 1..21: mol_type = other RNA
ctttttacagccaacttttct

SEQ ID NO: 94 - RNA, 21 nt, synthetic construct
  misc_feature 1..21: note = Description of Artificial Sequence: Synthetic oligonucleotide
  source 1..21: mol_type = other RNA
ctgttttgcttttgtaacttg

SEQ ID NO: 95 - RNA, 21 nt, synthetic construct
  misc_feature 1..21: note = Description of Artificial Sequence: Synthetic oligonucleotide
  source 1..21: mol_type = other RNA
gcttttgtaacttgaagatat

SEQ ID NO: 96 - RNA, 21 nt, synthetic construct
  misc_feature 1..21: note = Description of Artificial Sequence: Synthetic oligonucleotide
  source 1..21: mol_type = other RNA
cttttgtaacttgaagatatt

SEQ ID NO: 97 - RNA, 21 nt, synthetic construct
  misc_feature 1..21: note = Description of Artificial Sequence: Synthetic oligonucleotide
  source 1..21: mol_type = other RNA
gtaacttgaagatatttattc

SEQ ID NO: 98 - RNA, 21 nt, synthetic construct
  misc_feature 1..21: note = Description of Artificial Sequence: Synthetic oligonucleotide
  source 1..21: mol_type = other RNA
gggttttgtagcatttttatt

SEQ ID NO: 99 - RNA, 21 nt, synthetic construct
  misc_feature 1..21: note = Description of Artificial Sequence: Synthetic oligonucleotide
  source 1..21: mol_type = other RNA
ggttttgtagcatttttatta

SEQ ID NO: 100 - RNA, 21 nt, synthetic construct
  misc_feature 1..21: note = Description of Artificial Sequence: Synthetic oligonucleotide
  source 1..21: mol_type = other RNA
gttttgtagcatttttattaa

SEQ ID NO: 101 - RNA, 21 nt, synthetic construct
  misc_feature 1..21: note = Description of Artificial Sequence: Synthetic oligonucleotide
  source 1..21: mol_type = other RNA
gtagcatttttattaatatgg

SEQ ID NO: 102 - RNA, 21 nt, synthetic construct
  misc_feature 1..21: note = Description of Artificial Sequence: Synthetic oligonucleotide
  source 1..21: mol_type = other RNA
gcatttttattaatatggtga

SEQ ID NO: 103 - DNA, 3637 bp, Homo sapiens
  source 1..3637: mol_type = unassigned DNA
agcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaa

SEQ ID NO: 104 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide attached to aminohexyl-N-acetylgalactosamine x3
  misc_feature 1..10: note = RNA
  misc_feature 11: note = DNA
  misc_feature 12..21: note = RNA
ctagacctgttttgcttttgt

SEQ ID NO: 105 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage attached to N-acetylgalactosamine x3-Peptide docking vehicle 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
  misc_feature 1..10: note = RNA
  misc_feature 11: note = DNA
  misc_feature 12..21: note = RNA
ctagacctgttttgcttttgt

SEQ ID NO: 106 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide attached to (Trifluoroacetylamino)-hexyl-(2-cyanoethyl)-(N,N-diisoprop yl)-phosphoramidite-6-oxo-6-(dibenzo[b,f]azacyclooct-4-yn- 1-yl)-caproic acid sulfo-N-hydroxysuccinimide ester, sodium salt-Peptide docking vehicle 3-N-acetylgalactosamine x3
  misc_feature 1..10: note = RNA
  misc_feature 11: note = DNA
  misc_feature 12..21: note = RNA
ctagacctgttttgcttttgt

SEQ ID NO: 107 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide attached to N-acetylgalactosamine x3-Peptide docking vehicle 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-fluoro modified nucleotide
gcctggagtttattcggaa

SEQ ID NO: 108 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-O-methyl modified nucleotide attached to N-acetylgalactosamine x3-Peptide docking vehicle 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
gcctggagtttattcggaa

SEQ ID NO: 109 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-O-methyl modified nucleotide attached to N-acetylgalactosamine x3-Peptide docking vehicle 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
tcataggcctggagtttat

SEQ ID NO: 110 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-O-methyl modified nucleotide attached to N-acetylgalactosamine x3-Peptide docking vehicle 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
ggctgagctttaaaatggt

SEQ ID NO: 111 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide attached to N-acetylgalactosamine x3-Peptide docking vehicle 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide
tcataggcctggagtttat

SEQ ID NO: 112 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide with phosphorothioate linkage attached to N-acetylgalactosamine x3-Peptide docking vehicle 2-Serinol
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
gcctggagtttattcggaa

SEQ ID NO: 113 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-fluoro modified nucleotide with phosphorothioate linkage attached to N-acetylgalactosamine x3-Peptide docking vehicle 2-Serinol
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  misc_feature 1..19: note = RNA
  misc_feature 20..21: note = DNA
taacttgaaaatatttatatt

SEQ ID NO: 114 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide with phosphorothioate linkage attached to N-acetylgalactosamine x3-Peptide docking vehicle 3-Serinol
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
gcctggagtttattcggaa

SEQ ID NO: 115 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-fluoro modified nucleotide with phosphorothioate linkage attached to N-acetylgalactosamine x3-Peptide docking vehicle 3-Serinol
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide
  misc_feature 1..19: note = RNA
  misc_feature 20..21: note = DNA
taacttgaagatatttatatt

SEQ ID NO: 116 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide attached to N-acetylgalactosamine x3-Control 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-fluoro modified nucleotide
gcctggagtttattcggaa

SEQ ID NO: 117 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-O-methyl modified nucleotide attached to N-acetylgalactosamine x3-Control 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
gcctggagtttattcggaa

SEQ ID NO: 118 - RNA, 18 nt, synthetic construct
  misc_feature 1..18: note = Description of Artificial Sequence: Synthetic oligonucleotide
  source 1..18: mol_type = other RNA
  modified_base 1: mod_base = OTHER; note = 2'-O-methyl modified nucleotide attached to N-acetylgalactosamine x3-Peptide docking vehicle 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
cagggtatcgattacaaa

SEQ ID NO: 119 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-fluoro modified nucleotide attached to N-acetylgalactosamine x3-Control 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide
tcataggcctggagtttat

SEQ ID NO: 120 - RNA, 23 nt, synthetic construct
  misc_feature 1..23: note = Description of Artificial Sequence: Synthetic oligonucleotide
  source 1..23: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = 2'-O-methyl modified nucleotide
acaaaagcaaaacaggtctagaa

SEQ ID NO: 121 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
ttccgaataaactccaggctt

SEQ ID NO: 122 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide with phosphorothioate linkage
  modified_base 18: mod_base = OTHER; note = 2'-O-methyl modified nucleotide with phosphorothioate linkage
  modified_base 19: mod_base = OTHER; note = 2'-O-methyl modified nucleotide
  misc_feature 1..19: note = RNA
  misc_feature 20..21: note = DNA
ataaactccaggcctatgatt

SEQ ID NO: 123 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide with phosphorothioate linkage
  modified_base 19: mod_base = OTHER; note = 2'-O-methyl modified nucleotide with phosphorothioate linkage
  misc_feature 1..19: note = RNA
  misc_feature 20..21: note = DNA
accattttaaagctcagcctt

SEQ ID NO: 124 - DNA, 21 bp, synthetic construct
  misc_feature 1..21: note = Description of Artificial Sequence: Synthetic oligonucleotide
  source 1..21: mol_type = other DNA
  modified_base 1: mod_base = OTHER; note = Phosphorylated 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide
  misc_feature 1..19: note = RNA
  misc_feature 20..21: note = DNA
ataaactccaggcctatgatt

SEQ ID NO: 125 - RNA, 21 nt, synthetic construct
  misc_feature 1..21: note = Description of Artificial Sequence: Synthetic oligonucleotide
  source 1..21: mol_type = other RNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
tttgtaatcgtcgataccctg

SEQ ID NO: 126 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-O-methyl modified nucleotide attached to N-acetylgalactosamine x3-Peptide docking vehicle 3-6-oxo-6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
gcctggagtttattcggaa

SEQ ID NO: 127 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
gcctggagtttattcggaa

SEQ ID NO: 128 - RNA, 19 nt, synthetic construct
  misc_feature 1..19: note = Description of Artificial Sequence: Synthetic oligonucleotide
  source 1..19: mol_type = other RNA
  modified_base 1: mod_base = OTHER; note = Phosphorylated 2'-fluoro modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
ttccgaataaactccaggc

SEQ ID NO: 129 - protein, 7 aa, synthetic construct
  SITE 1: note = polyethylene glycol 4-azide attached to side chain of Lysine
  SITE 5: note = polyethylene glycol 6-N-acetylgalactosamine x3 attached to side chain of Cysteine
KHHHCHH

SEQ ID NO: 130 - protein, 11 aa, synthetic construct
HHHKHHCKHHH

SEQ ID NO: 131 - protein, 7 aa, synthetic construct
  VARIANT 6: note = Any amino acid
HKHHCXH

SEQ ID NO: 132 - protein, 7 aa, synthetic construct
KHHHCHH

SEQ ID NO: 133 - protein, 6 aa, synthetic construct
  VARIANT 5: note = K or R
HKHCXH

SEQ ID NO: 134 - protein, 7 aa, synthetic construct
  SITE 1: note = polyethylene glycol 4-Bis-NHS-(Trifluoroacetylamino )-hexyl-(2-cyanoethyl)-(N,N-diisopropyl)-phosphoramidite modified residue
  SITE 5: note = trivalent beta-N-Acetylgalactosamine polyethylene glycol maleimide modified residue
  VARIANT 6: note = H, K or R
KHHHCXH

SEQ ID NO: 135 - protein, 7 aa, synthetic construct
  SITE 1: note = polyethylene glycol 4-azide-dibenzocyclooctyl-(Trif luoroacetylamino)-hexyl-(2-cyanoethyl)-(N,N-diisopropyl)-p hosphoramidite modified residue
  SITE 5: note = trivalent beta-N-Acetylgalactosamine polyethylene glycol maleimide modified residue
  SITE 6: note = polyethylene glycol 4-azide-dibenzocyclooctyl-(Trif luoroacetylamino)-hexyl-(2-cyanoethyl)-(N,N-diisopropyl)-p hosphoramidite modified residue
KHHHCKH

SEQ ID NO: 136 - protein, 11 aa, synthetic construct
  SITE 4: note = polyethylene glycol 4-azide attached to side chain of Lysine
  SITE 7: note = trivalent beta-N-Acetylgalactosamine polyethylene glycol maleimide modified residue
HHHKHHCKHHH

SEQ ID NO: 137 - protein, 7 aa, synthetic construct
  SITE 1: note = polyethylene glycol 4-azide attached to side chain of Lysine
KHHHCHH

SEQ ID NO: 138 - protein, 11 aa, synthetic construct
SSSKSSCRSSS

SEQ ID NO: 139 - protein, 7 aa, synthetic construct
KSSSCSS

SEQ ID NO: 140 - protein, 7 aa, synthetic construct
  VARIANT 6: note = Any amino acid
HRHHCXH

SEQ ID NO: 141 - protein, 5 aa, synthetic construct
HKHCH

SEQ ID NO: 142 - protein, 4 aa, synthetic construct
HKHC

SEQ ID NO: 143 - protein, 7 aa, synthetic construct
  SITE 1: note = polyethylene glycol 4-azide-dibenzocyclooctyl-(Trif luoroacetylamino)-hexyl-(2-cyanoethyl)-(N,N-diisopropyl)-p hosphoramidite modified residue
  SITE 5: note = trivalent beta-N-Acetylgalactosamine polyethylene glycol maleimide modified residue
  VARIANT 6: note = K or R
KHHHCXH

SEQ ID NO: 144 - protein, 11 aa, synthetic construct
  SITE 4: note = polyethylene glycol 4-azide-dibenzocyclooctyl-(Trif luoroacetylamino)-hexyl-(2-cyanoethyl)-(N,N-diisopropyl)-p hosphoramidite modified residue
  SITE 7: note = trivalent beta-N-Acetylgalactosamine polyethylene glycol maleimide modified residue
  VARIANT 8: note = K or R
HHHKHHCXHHH